(19) United States
(12) Patent Application Publication
(10) Pub. No.: US 2006/0089371 A1
(43) Pub. Date: Apr. 27, 2006

(54) Phenyl or heteroaryl amino alkane derivatives as ip receptor antagonist

(75) Inventors: Toshiki Murata, Nara (JP); Masami Umeda, Shiga-ken (JP); Satoru Yoshikawa, Kanagawa-ken (JP); Klaus Urbahns, Lund (SE); Jang Gupta, Dusseldorf (DE); Osamu Sakurai, Kyoto (JP)

Correspondence Address: JEFFREY M. GREENMAN, BAYER PHARMACEUTICALS CORPORATION, 400 MORGAN LANE, WEST HAVEN, CT, 06516, US

(73) Assignee: Bayer HealthCare AG, Leverkusen (DE)

(21) Appl. No.: 10/534,174

(22) PCT Filed: Oct. 29, 2003
(86) PCT No.: PCT/EP03/11976

§ 371 Date: May 6, 2005

(30) Foreign Application Priority Data
Nov. 11, 2002 (EP) 02025024.7
May 20, 2003 (EP) 03011397.1

(51) Int. Cl. A61K 31/506; C07D 403/02

(52) U.S. Cl. 514/256; 544/333; 544/330

(57) ABSTRACT

The present invention relates to phenyl or heteroaryl amino alkane derivatives of formula (I) 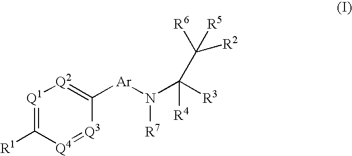
in which the groups Q¹-Q⁴, Ar, and R¹-R⁷ are as defined in the specification and claims. These materials are useful as active ingredients of pharmaceutical preparations. The phenyl or heteroaryl amino alkanes of the present invention have IP receptor antagonistic activity, and can be used for the prophylaxis and treatment of diseases associated with IP receptor antagonistic activity. Such diseases include urological diseases or disorders as follows: bladder outlet obstruction, overactive bladder, urinary incontinence, detrusor hyper-reflexia, detrusor instability, reduced bladder capacity, frequency of micturition, urge incontinence, stress incontinence, bladder hyperreactivity, benighn prostatic hypertrophy (BPH), prostatitis, urinary frequency, nocturia, urinary urgency, pelvic hypersensitivity, urethritis, pelvic pain syndrome, prostatodynia, cystitis, or idiophatic bladder hypersensitivity. The compounds of the present invention are also useful for treatment of pain including, but not limited to inflammatory pain, neuropathic pain, acute pain, chronic pain, dental pain, premenstrual pain, visceral pain, headaches, and the like; hypotension; hemophilia and hemorrhage; and inflammation, since these diseases also are alleviated by treatment with an IP receptor antagonist. The application claims the compounds, pharmaceutical compositions containing them, and methods of treatment using them.

### DETAILED DESCRIPTION OF INVENTION

1. Technical Field

The present invention relates to a phenyl or heteroaryl amino alkane derivatives which are useful as an active ingredient of pharmaceutical preparations. The phenyl or heteroaryl amino alkane derivatives of the present invention have IP receptor antagonistic activity, and can be used for the prophylaxis and treatment of diseases associated with IP receptor antagonistic activity.

More specifically, the phenyl or heteroaryl amino alkane derivatives of the present invention are useful for treatment and prophylaxis of urological diseases or disorders.

The compounds of the present invention are also useful for treatment of pain; hypotension; hemophilia and hemorrhage; inflammation; respiratory states from allergies or asthma, since the diseases also is alleviated by treatment with an IP receptor antagonist.

2. Background Art

Prostaglandins (or prostanoids, PGs) are a group of bioactive lipid mediators generated from membrane phospholipids. They are formed from 20-carbon essential fatty acids containing 3, 4, or 5 double bonds, and carry a cyclopentane ring. They are divided into 6 main classes (D, E, F, G, H or I) by the cyclopentane ring structure. The main classes are further subdivided by subscripts 1, 2, or 3, reflecting their fatty acid precursors. PGI2 is a member of prostanoids, and it has a double ring structure and is derived from arachidonic acid. The receptor for PGI2 is a seven transmembrane G-protein coupled receptor, called prostacyclin receptor (IP). IP couples at least to Gs-type G-protein, and activates adenylate cyclase and phospholipase C. The expression of IP is demonstrated in aorta, coronary/pulmonary/cerebral arteries, platelets, lung, and dorsal root ganglions in addition to several other tissues.

One of the well-known actions of PGI2 on blood vessels is to cause vasodilation and hypotension. Especially in septic shock, PGI2 is produced and participates in the induction of systemic hypotension (G. D. Bottoms et al, Am J Vet Res 1982, 43(6), 999-1002). Therefore, IP receptor antagonists may prevent hypotension associated with septic shock.

Another well-known action of PGI2 on platelets is to suppress aggregation. In the IP receptor knock out mice, FeCl₃-induced thrombosis formation was enhanced in comparison with that in wild type mice (T. Murata et al, Nature 1997, 388, 678-682), confirming the involvement of IP receptor in the platelet inhibition. Therefore, IP receptor antagonists may enhance the platelet activation and suppress excessive bleeding such as, but not limited to, hemophilia and hemorrhage.

PGI2 also participates in the inflammation. In the inflamed tissue, various inflammatory mediators, including prostaglandins, are produced. PGI2 is also generated and induces vasodilation to increase blood flow. This enhances vascular permeability, edema formation and leukocyte inflammation in the inflamed region (T. Murata et al, Nature 1997, 388, 678-682). Therefore, PGI2 receptor antagonists may be efficacious for the treatment of inflammation.

PGI2 may be involved in the pathogenesis of respiratory allergy or asthma. It is spontaneously generated and the major prostaglandin in human lung, and the appropriate antigen challenge increases PGI2 production (E. S. Schulman et al, J Appl Physiol 1982, 53(3), 589-595). Therefore, IP antagonists may have a utility for the treatment of those respiratory diseases.

In addition, an important role of IP receptor in the induction of hyperalgesia has been clearly shown by IP receptor knockout mice (T. Murata et al., Nature 1997, 388, 678-682.). Injection of acetic acid into the peritoneal cavity induced production of PGI2. This PGI2 is considered to bind to IP receptor on sensory neurons. As IP receptor couples to the activation of both adenylate cyclase and phospholipase C, cAMP-dependent protein kinase (PKA) and protein kinase C (PKC) are activated. PKA and PKC are known to modulate ion channels on sensory neurons such as VR1, P2X3, and TITX-R As a result, PGI2 sensitizes sensory neurons to enhance the release of neurotransmitters. An acetic acid injection induces nociceptive response (writhing) in mice and this acetic acid-induced writhing was greatly reduced in IP receptor-null mice as the same level as indomethacin-treated wild type mice. Several other in vivo hyperalgesia studies in rodents and in vitro studies further support that PGI2 plays a major role in the induction of hyperalgesia and that PGI2 acts as important modulator of sensory neurons (K. Bley et al, Trends in Pharmacological Sciences 1998, 19(4), 141-147). Therefore, IP receptor antagonists may be useful for the treatment of pain.

Sensory neurons play very important roles not only in the pain sensation but also in the sensation of bladder distension. In normal subjects, A-delta sensory fibers are considered to play a major role to sense the bladder distention. However, in disease conditions of overactive bladder by, but not limited to, spinal cord injury, cystitis, Parkinson's disease, multiple sclerosis, previous cerebrovascular accident, and bladder outlet obstruction (BOO) caused by benign prostate hyperplasia (BPH), the sensitivity of C-fiber sensory neurons is upregulated and they contribute to the induction of the lower urinary tract symptoms. Treatment of overactive bladder patients with intravesical injection of capsaicin or its potent analog, resiniferatoxin, both of which desensitize VR1-positive C-fiber afferent neurons innervating the bladder, has been shown to be efficacious in several clinical trials (C. Silva et al, Eur Urol. 2000, 38(4), 444-452). Therefore, C-fiber sensory neurons play an important role in the pathology of overactive bladder. PGI2 is generated locally in the bladder and it is the major prostaglandin released from the human bladder. In a rabbit BOO model, a stable metabolite of PGI2 was reported to be increased in BOO bladder (J M. Masick et al, Prostaglandins Other Lipid Mediat. 2001, 66(3), 211-219). Hence, PGI2 from disease bladder sensitizes C-fiber sensory neurons, and as a result, it may induce symptoms of overactive bladder. Therefore, antagonists of IP receptor are expected to be useful in the treatment of overactive bladder and related urinary disorders.

WO 00/43369 discloses pharmaceutical composition intended for the treatment of immune or inflammatory disorders represented by the general formula: 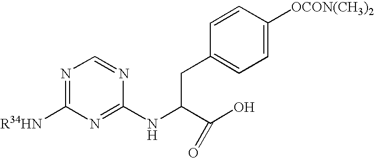
wherein
R³⁴ is optionally substituted alkyl, optionally substituted aryl or optionally substituted heteroaryl.

However, none of the references and other reference discloses phenyl or heteroaryl amino alkane derivatives having IP receptor antagonistic activity.

The development of a compound which has effective IP receptor antagonistic activity and can be used for the prophylaxis and treatment of diseases associated with IP receptor antagonistic activity, has been desired.

### SUMMARY OF THE INVENTION

As the result of extensive studies on chemical modification of phenyl or heteroaryl amino alkane derivatives, the present inventors have found that the compounds of the structure related to the present invention have unexpectedly excellent IP receptor antagonistic activity. The present invention has been accomplished based on these findings.

This invention is to provide a novel phenyl or heteroaryl amino alkane derivative of the formula (I), its tautomeric or stereoisomeric form, or a salt thereof: 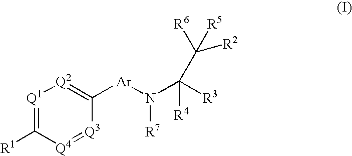
wherein
Ar represents phenylene or a 5 or 6 membered heteroaryl containing 1-3 heteroatoms selected from the group consisting of O, N and S,
   wherein
   said phenyl or a 5 or 6 membered heteroaryl optionally having one or more substituents selected from the group consisting of halogen, hydroxy, cyano, nitro, amino, N-(C₁₋₆)alkylamino, N,N-di(C₁₋₆)alkylamino, formyl, (C₁₋₆)alkylthio, (C₁₋₆)alkoxy and (C₁₋₆)alkyl optionally substituted by hydroxy, or mono-, di- or tri-halogen;
Q¹, Q², Q³ and Q⁴ independently represent CH, CR¹⁰ or N;
   wherein
   R¹⁰ represents halogen, cyano, amino, nitro, formyl, hydroxymethyl, methylthio, (C₁₋₆)alkyl optionally substituted by mono-, di- or trihalogen, or (C₁₋₆)alkoxy optionally substituted by phenyl;
R¹ represents —OR¹¹, —CH₂NHR¹¹, —C(O)R¹¹, —C(O)NHR¹¹, —SR¹¹, —SOR¹¹, —SO₂R¹¹, —NHR¹¹, —NHC(O)OR¹¹, —NHC(O)NR¹¹, —NHC(O)R¹¹, —NHSO₂R¹¹, hydrogen, hydroxy, halogen,
   a saturated or unsaturated 3-10 membered mono- or bi-cyclic ring optionally having one or two heteroatoms selected independently from O or N,
   (C₁₋₆)alkyl optionally substituted by aryloxyimino, (C₁₋₆)alkoxy optionally substituted by aryl or heteroaryl, or
   a saturated or unsaturated 3-10 membered mono- or bi-cyclic ring optionally having one or two heteroatoms selected independently from O or N,
   (C₂₋₆)alkenyl optionally substituted by a saturated or unsaturated 3-10 membered mono- or bi-cyclic ring optionally having one or two heteroatoms selected independently from O or N,
   (C₂)alkynyl optionally substituted by a saturated- or unsaturated 3-10 membered mono- or bi-cyclic ring optionally having one or two heteroatoms selected independently from O or N,
   in any of which the saturated or unsaturated 3-10 membered mono- or bi-cyclic ring may be optionally substituted by one or more substituents selected from the group consisting of
      halogen, hydroxy, cyano, nitro, (C₁₋₆)alkylthio,
      (C₁₋₆)alkyl optionally substituted by mono-, di-, or tri-halogen,
      (C₁₋₆)alkoxy optionally substituted by mono-, di-, or tri-halogen,
      aryl optionally substituted by nitro, (C₁₋₆)alkyl or (C₁₋₆)alkoxy,
      aralkyl optionally, at the aryl moiety, substituted by nitro, (C₁₋₆)alkyl or (C₁₋₆)alkoxy,
      and
      aryloxy optionally substituted by nitro, (C₁₋₆)alkyl or (C₁₋₆)alkoxy,
      wherein
      R¹¹ represents (C₁₋₆)alkoxy(C₁₋₆)alkylene,
         a saturated or unsaturated 3-10 membered mono- or bi-cyclic ring optionally having one or two heteroatoms selected independently from O or N,
         (C₁₋₆)alkyl optionally substituted by mono-, di- or tri-halogen or a saturated or unsaturated 3-10 membered mono- or bi-cyclic ring optionally having one or two heteroatoms selected independently from O or N,
         (C₂₋₆)alkenyl optionally substituted by a saturated or unsaturated 3-10 membered mono- or bi-cyclic ring optionally having one or two heteroatoms selected independently from O or N, or
         (C₂₋₆)alkynyl optionally substituted by a saturated or unsaturated 3-10 membered mono- or bi-cyclic ring optionally having one or two heteroatoms selected independently from O or N,
         in any of which the saturated or unsaturated 3-10 membered mono- or bi-cyclic ring may be optionally substituted by one or more substituents selected from the group consisting of
            halogen, hydroxy, cyano, nitro,
            (C₁₋₆)alkoxy optionally substituted by mono-, di-, or tri-halogen, and
            (C₁₋₆)alkyl optionally substituted by mono-, di-, or tri-halogen;
R² represents hydrogen, hydroxy, amino, N-(C₁₋₆)alkylamino, (C₂₋₆)alkenyl, (C₂₋₆)alkynyl, (C₃₋₇)cycloalkyl, (C₁₋₆)alkylthio, (C₁₋₆)alkylsulfonyl, aryl, heteroaryl,
   (C₁₋₆)alkyl optionally substituted by mono-, di- or tri-halogen, (C₁₋₆)alkylsulfonyl, (C₁₋₆)alkylthio, aryl or heteroaryl, or
   (C₁₋₆)alkoxy optionally substituted by mono-, di- or tri-halogen, (C₁₋₆)alkylsulfonyl, aryl or heteroaryl,
   in any of which the aryl, or heteroaryl may optionally be substituted by one or more substituents selected from the group consisting of halogen, hydroxy, nitro, amino, N-(C₁₋₆)alkylamino, N,N-di(C₁₋₆)alkylamino, N-(4,5-dihydro-1H-imidazole)amino, (C₁₋₆)alkyl, phenyl, a 5 or 6 membered heteroaryl containing 1 to 3 heteroatoms selected from the group of O, N, and S,
   and
   (C₁₋₆)alkoxy optionally substituted by morpholino, amino, N-(C₁₋₆)alkylamino, or N,N-di(C₁₋₆) alkylamino;
R³ represents hydrogen, or C₁₋₆ alkyl optionally substituted mono-, di- or tri-halogen;
R⁴ represents carboxy, tetrazolyl or N-(hydroxy)aminocarbonyl;
R⁵ represents hydrogen, (C₁₋₆)alkoxy, aryl, heteroaryl or (C₁₋₆)alkyl optionally substituted by mono-, di- or tri-halogen;
R⁶ represents hydrogen or (C₁₋₆)alkyl optionally substituted by mono-, di- or tri-halogen; and
R⁷ represents hydrogen, or (C₁₋₆)alkyl.

The compounds of the present invention surprisingly show excellent IP receptor antagonistic activity. They are, therefore, suitable for the production of medicament or medical composition, which may be useful for diseases, is alleviated by treatment with an IP receptor antagonist.

More specifically, since the carboxamides derivatives of the present invention antagonize IP receptor, they are useful for treatment and prophylaxis of urological diseases or disorder.

The compounds of the present invention are also useful for treatment of urological diseases or disorders. Such diseases or disorders include bladder outlet obstruction, overactive bladder, urinary incontinence, detrusor hyper-reflexia, detrusor instability, reduced bladder capacity, frequency of micturition, urge incontinence, stress incontinence, bladder hyperreactivity, benighn prostatic hypertrophy (BP), prostatitis, urinary frequency, nocturia, urinary urgency, pelvic hypersensitivity, urethritis, pelvic pain syndrome, prostatodynia, cystitis, or idiophatic bladder hypersensitivity.

The compounds of the present invention are also useful for treatment of pain including, but not limited to inflammatory pain, neuropathic pain, acute pain, chronic pain, dental pain, premenstrual pain, visceral pain, headaches, and the like; hypotension; hemophilia and hemorrhage; inflammation; respiratory states from allegies or asthma, since the diseases which are alleviated by treatment with IP receptor antagonist.

Yet another embodiment of the compounds of formula (I) are those wherein:
Ar represents 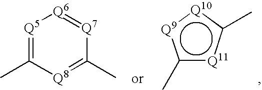
   Q⁵, Q⁶, Q⁷ and Q⁸ independently represent CH, CR⁸ or N,
   Q⁹, Q¹⁰ and Q¹² independently represent O, S, CH, CR⁸, CH₂, NH, or NR⁹,
      wherein
      R⁸ represents halogen, cyano, amino, nitro, formyl hydroxymethyl, methylthio, (C₁₋₆)alkoxy, or (C₁₋₆)alkyl optionally substituted by mono-, di- or tri-halogen,
      R⁹ represents (C₁₋₆)alkyl;
Q¹, Q², Q³ and Q⁴ independently represent CH, CR¹⁰ or N,
   wherein
   R¹⁰ represents halogen, amino, nitro, formyl, hydroxymethyl, methylthio, (C₁₋₆)alkyl optionally substituted by mono-, di- or tri-halogen, or (C₁₋₆)alkoxy optionally substituted by phenyl;
R¹ represents —OR¹¹, —CH₂NHR¹¹, —C(O)R¹¹, —C(O)NHR¹¹, —SR¹¹, —SOR¹¹, —SO₂R¹¹, —NHR¹¹, —NHC(O)R¹¹, —NHC(O)OR¹¹, —NHC(O)N¹¹, —NHSO₂R¹¹, hydrogen, hydroxy, halogen,
   a saturated or unsaturated 3-10 membered mono-, or bi-cyclic ring optionally having one or two heteroatoms selected independently from O or N,
   (C₁₋₆)alkyl optionally substituted by aryloxyimino, (C₁₋₆) alkoxy optionally substituted by aryl or hereoaryl, or a saturated or unsaturated 3-10 membered mono- or bi-cyclic ring optionally having one or two heteroatoms selected independently from O or N,
   (C₂₋₆)alkenyl optionally substituted by a saturated or unsaturated 3-10 membered mono- or bi-cyclic ring optionally having one or two heteroatoms selected independently from O or N,
   (C₂₋₆)alkyl optionally substituted by a saturated or unsaturated 3-10 membered mono- or bi-cyclic ring optionally having one or two heteroatoms selected independently from O or N,
   in any of which the saturated or unsaturated 3-10 membered mono- or bi-cyclic ring may be optionally substituted by one or more substituents selected from the group consisting of
      halogen, hydroxy, cyano, nitro, (C₁₋₆) alkylthio,
      (C₁₋₆)alkyl optionally substituted by mono-, di-, or tri-halogen,
      (C₁₋₆)alkoxy optionally substituted by mono-, di-, or tri-halogen,
      aryl optionally substituted by nitro, (C₁₋₆) alkyl or (C₁₋₆) alkoxy,
      aralkyl optionally, at the aryl moiety, substituted by nitro, (C₁₋₆)alkyl or (Cl₆)alkoxy,
      and
      aryloxy optionally substituted by nitro, (C₁₋₆)alkyl or (C₁₋₆)alkoxy,
      wherein
      R¹¹ represents (C₁₋₆)alkoxy(C₁₋₆)alkylene,
         a saturated or unsaturated 3-10 membered mono- or bi-cyclic ring optionally having one or two heteroatoms selected independently from O or N,
         (C₁₋₆)alkyl optionally substituted by mono-, di- or tri-halogen or a saturated or unsaturated 3-10 membered mono- or bi-cyclic ring optionally having one or two heteroatoms selected independently from O or N.
         (C₂₋₆)alkenyl optionally substituted by a saturated or unsaturated 3-10 membered mono- or bi-cyclic ring optionally having one or two heteroatoms selected independently from O or N, or
         (C₂₋₆)alkynyl optionally substituted by a saturated or unsaturated 3-10 membered mono- or bi-cyclic ring optionally having one or two heteroatoms selected independently from O or N,
         in any of which the saturated or unsaturated 3-10 membered mono- or bi-cyclic ring may be optionally substituted by one or more substituents selected from the group consisting of
            halogen, hydroxy, cyano, nitro,
            (C₁₋₆)alkoxy optionally substituted by mono-, di-, or tri-halogen, and
            (C₁₋₆)alkyl optionally substituted by mono-, di-, or tri-halogen;
R² represents hydrogen, hydroxy, amino, N-(C₁₋₆)alkylamino, (C₂₋₆)alkenyl, (C₂₋₆)alkyl, (C₃₋₇)cycloalkyl, (C₁₋₆)alkylthio, (C₁₋₆)alkylsulfonyl, aryl, heteroaryl,
   (C₁₋₆)alkyl optionally substituted by mono-, di- or tri-halogen, (C₁₋₆)alkyl-sulfonyl, (C₁₋₆)alkylthio, aryl or heteroaryl, or
   (C₁₋₆)alkoxy optionally substituted by mono-, di- or tri-halogen, (C₁₋₆)alkyl-sulfonyl, aryl or heteroaryl,
   in any of which the aryl or heteroaryl may optionally be substituted by one or more substituents selected from the group consisting of halogen, hydroxy, nitro, amino, N-(C₁₋₆)alkylamino, N,N-di(C₁₋₆) alkylamino, N-(4,5-dihydro-1H-imidazole)amino, (C₁₋₆)alkyl, phenyl, a 5 or 6 membered heteroaryl containing 1 to 4 heteroatoms selected from the group of O, N, and S,
   and
   (C₁₋₆)alkoxy optionally substituted by morpholino, amino, N-(C₁₋₆)alkylamino, or N,N-di(C₁₋₆) alkylamino;
R³ represents hydrogen, or C₁₋₆alkyl optionally substituted mono, di- or tri-halogen;
   R⁴ represents carboxy, tetrazolyl or N-(hydroxy)aminocarbonyl; R⁵ represents hydrogen, (C₁₋₆)alkyl, (C₁₋₆)alkoxy, aryl or heteroaryl;
R⁶ represents hydrogen; and
R⁷ represents hydrogen, or (C₁₋₆)alkyl.

Another embodiment of the compounds of formula (I) is those wherein:
Ar represents 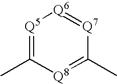
   Q⁵, Q⁶, Q⁷ and Q⁸ independently represent CH, CR⁸ or N,
      wherein
      R⁸ represents halogen, cyano, amino, nitro, formyl, hydroxymethyl, methylthio, (C₁₋₆)alkoxy, or (C₁₋₆)alkyl optionally substituted by mono-, di-, or tri-halogen;
   Q¹, Q², Q³ and Q⁴ independently represent CH, CR¹⁰ or N,
      wherein
      R¹⁰ represents halogen, amino, nitro, formyl, trifluoromethyl, hydroxymethyl, methylthio or benzyloxy;
R¹ represents —OR¹¹, —CH₂OR¹¹, —CH₂NHR¹¹, —C(O)R¹¹, —C(O)NHR¹¹, —SR¹¹, —SOR¹¹, —SO₂R¹¹, —NHR¹¹, —NHC(O)R¹¹, —NHC(O)OR¹¹, —NHC(O)NR¹¹, —NHSO₂R¹¹, hydrogen, hydroxy, halogen,
   (C₁₋₆) alkyl optionally substituted by phenoxyimino, (C₁₋₆) alkoxy or R¹²,
      wherein
      said (C₁₋₆) alkoxy optionally substituted by pyrrolyl, pyrazolyl, imidazolyl, phenyl, pyridyl, pyrazinyl, pyridazinyl, pyrimidinyl benzodioxolyl naphthyl indolyl, isoindolyl quinolyl isoquinolyl, or dihydroisoquinolyl,
   (C₂₋₆)alkenyl optionally substituted by R¹²,
   (C₂₋₆)alkynyl optionally substituted by R¹², or
   one of the following carbocyclic or heterocyclic rings selected from the group consisting of cyclopropyl, cyclobutyl, cyclopentyl, cyclohexyl, pyrrolidinyl pyrrolyl, piperidino, piperidyl, piperazinyl, pyrazolyl, imidazolyl, phenyl pyridyl, pyrazinyl, pyridazinyl, pyrimidinyl, benzodioxolyl, naphthyl, indolyl, isoindolyl, quinolyl, isoquinolyl, and dihydroisoquinolyl,
   in any of which carbocyclic or heterocyclic rings may optionally be substituted with 1 to 3 substituents selected from the group consisting of hydroxy, halogen, nitro, cyano, carboxy, amino, N-(C₁₋₆)alkylamino, N,N-di(C₁₋₆)alkylamino, (C₁₋₆)alkylthio, phenyl, phenoxy, benzyl, naphthyl, (C₁₋₆)alkyl-optionally substituted by mono-, di- or tri-halogen, and (C₁₋₆)alkoxy optionally substituted by mono-, di- or tri-halogen, or phenyl;
   wherein
   R¹¹ represents (C₁₋₆)alkoxy(C₁₋₆)alkylene,
      (C₁₋₆)alkyl optionally substituted by R¹⁰¹,
      (C₂₋₆)alkenyl optionally substituted by R¹⁰¹,
      (C₂₋₆)alkynyl optionally substituted by R¹⁰¹, or
      one of the following carbocyclic or heterocyclic rings selected from the group consisting of cyclopropyl, cyclobutyl, cyclopentyl, cyclohexyl, pyrrolidinyl pyrrolyl, piperidino, piperidyl, piperazinyl, pyrazolyl, imidazolyl, phenyl, pyridyl, pyrazinyl, pyridazinyl, pyrimidinyl, benzodioxolyl, naphthyl, indolyl, isoindolyl, quinolyl, isoquinolyl, and dihydroisoquinolyl,
      in any of which the carbocyclic or heterocyclic rings may optionally be substituted with 1 to 3 substituents selected from the group consisting of hydroxy, halogen, nitro, cyano, carboxy, amino, N-(C₁, alkyl)amino, N,N-di(C₁₋₆alkyl)amino, (C₁₋₆)alkylthio, phenyl, phenoxy, benzyl, naphthyl, (C₁₋₆)alkyl optionally substituted by mono-, di- or tri-halogen, and (C₁₋₆)alkoxy optionally substituted by mono-, di- or tri-halogen,
      R¹⁰¹ represents one of the following carbocyclic or heterocyclic rings selected from the group consisting of cyclopropyl, cyclobutyl, cyclopentyl, cyclohexyl, pyrrolidinyl, pyrrolyl, piperidino, piperidyl, piperazinyl, pyrazolyl, imidazolyl, phenyl, pyridyl, pyrazinyl, pyridazinyl, pyrimidinyl, benzodioxolyl, naphthyl, indolyl, isoindolyl, quinolyl, isoquinolyl, and dihydroisoquinolyl,
         in any of which the carbocyclic or heterocyclic rings may optionally be substituted with 1 to 3 substituents selected from the group consisting of hydroxy, halogen, nitro, cyano, carboxy, amino, N-(C₁₋₆alkyl)amino, N,N-di(C₁₋₆alkyl)amino, (C₁₋₆)alkylthio, phenyl, phenoxy, benzyl, naphthyl, (C₁₋₆)alkyl optionally substituted by mono-, di- or tri-halogen, and (C₁₋₆)alkoxy optionally substituted by mono-, di-, or tri halogen;
   R¹² represents one of the following carbocyclic or heterocyclic rings selected from the group consisting of cyclopropyl, cyclobutyl, cyclopentyl, cyclohexyl, pyrrolidinyl pyrrolyl, piperidino, piperidyl, piperazinyl, pyrazolyl, imidazolyl, phenyl, pyridyl, pyrazinyl, pyridazinyl, pyrimidinyl, benzodioxolyl, naphthyl, indolyl, isoindolyl, quinolyl, isoquinolyl, and dihydroisoquinolyl,
      in any of which the carbocyclic or heterocyclic rings may optionally be substituted with 1 to 3 substituents selected from the group consisting of hydroxy, halogen, nitro, cyano, carboxy, amino, N-(C₁₋₆alkyl)amino, N,N-di(C₁₋₆alky)amino, (C₁₋₆)alkylthio, phenyl, phenoxy, benzyl, naphthyl, (C₁₋₆)alkyl optionally substituted by mono-, di- or tri-halogen, and (C₁₋₆)alkoxy optionally substituted by mono-, di- or tri-halogen;
R² represents hydrogen, hydroxy, amino, N-(C₁₋₆)alkylamino, (C₂₋₆)alkyl, (C₂₋₆)alkynyl, (C₃₋₇)cycloalkyl, pyrimidinyl, indolyl, pyridyl,
   (C₁₋₆)alkoxy optionally substituted by amino, N-(C₁₋₆)alkylamino, N,N-di(C₁₋₆)alkylamino, or phenyl,
   (C₁₋₆) alkyl optionally substituted by phenyl, mono-, di- or tri-halogen, (C₁₋₆)alkylthio, or (C₁₋₆) alkylsulfonyl,
   phenyl optionally substituted by halogen, hydroxy, nitro, amino, N-(C₁₋₆)alkylamino, N-(dihydroimidazolyl)amino, (C₁₋₆)alkyl, or (C₁₋₆)alkoxy optionally substituted by R²¹,
   wherein
   R²¹ represents amino, N-(C₁₋₆)alkylamino, N,N-di(C₁₋₆)alkylamino, or morpholino;
R³ represents hydrogen, or (C₁₋₆)alkyl optionally substituted by mono-, di- or tri-halogen;
R⁴ represents carboxy, tetrazolyl or N-(hydroxy)aminocarbonyl;
R⁵ represents hydrogen, (C₁₋₆)alkyl, (C₁₋₆)alkoxy, phenyl, pyridyl, pyrazinyl, pyrimidinyl, or pyridazinyl;
R⁶ represents hydrogen; and
R⁷ represents hydrogen or (C₁₋₆)alkyl.

Another embodiment of the compounds of formula (I) is those wherein:
Ar represents 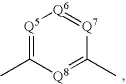
   Q⁵ and Q⁷ independently represent CH or N,
   Q⁶ and Q⁸ independently represent CH or CR⁸,
      wherein
      R⁸ represents halogen, cyano, amino, nitro, formyl, hydroxymethyl, methylthio or trifluoromethyl;
Q¹ independently represent represents CH or CR¹⁰,
   wherein
   R¹⁰ represents halogen, cyano, amino, nitro, formyl trifluoromethyl, hydroxymethyl, methylthio or benzyloxy;
   Q², Q³ and Q⁴ represent CH;
R¹ represents —OR¹¹, —CH₂NHR¹¹, —C(O)R¹¹, —C(O)NHR¹¹, —SR¹¹, —SOR¹¹, —SO₂R¹¹, —NHR¹¹, —NHC(O)R¹¹, —NHC(O)OR¹¹, —NH C(O)NR¹¹, —NHSO₂R¹¹, hydrogen, hydroxy, halogen,
   (C₁₋₆)alkyl optionally substituted by (C₁₋₆) alkoxy or R¹²,
      wherein
      said (C₁₋₆)alkoxy optionally substituted by pyrrolyl, pyrazolyl, imidazolyl, phenyl, pyridyl, pyrazinyl, pyridazinyl, pyrimidinyl, benzodioxolyl, naphthyl, indolyl, isoindolyl, quinolyl, isoquinolyl, or dihydroisoquinolyl,
   (C₂₋₆)alkenyl optionally substituted by R¹²,
   (C₂₋₆)alkynyl optionally substituted by R¹², or
   one of the following carbocyclic or heterocyclic rings selected from the group consisting of cyclopropyl, cyclobutyl, cyclopentyl, cyclohexyl, pyrrolidinyl pyrrolyl, piperidino, piperidyl, piperazinyl, pyrazolyl imidazolyl, phenyl, pyridyl, pyrazinyl, pyridazinyl pyrimidinyl, benzodioxolyl, naphthyl, indolyl, isoindolyl, quinolyl, isoquinolyl and dihydroisoquinolyl,
   in any of which the carbocyclic or heterocyclic rings may optionally be substituted with 1 to 3 substituents selected from the group consisting of hydroxy, halogen, nitro, cyano, amino, N-(C₁₋₆alkyl)amino, N,N-di(C₁₋₆alkyl)amino, (C₁₋₆)alkylthio, phenyl, phenoxy, benzyl, naphthyl,
   (C₁₋₆)alkyl optionally substituted by mono-, di- or tri-halogen, and (C₁₋₆)-alkoxy optionally substituted by mono-, di- or tri-halogen,
   wherein
   R¹¹ represents (C₁₋₆)alkoxy(C₁₋₆)alkylene,
      (C₁₋₆)alkyl optionally substituted by R¹⁰¹,
      (C₂₋₆)alkenyl optionally substituted by R¹⁰¹,
      (C₂₋₆)alkynyl optionally substituted by R¹⁰¹, or
      one of the following carbocyclic or heterocyclic rings selected from the group consisting of cyclopropyl, cyclobutyl, cyclopentyl, cyclohexyl, pyrrolidinyl pyrrolyl, piperidino, piperidyl, piperazinyl, pyrazolyl, imidazolyl, phenyl, pyridyl, pyrazinyl, pyridazinyl, pyrimidinyl, benzodioxolyl, naphthyl, indolyl, isoindolyl, quinolyl, isoquinolyl, and dihydroisoquinolyl,
      in any of which the carbocyclic or heterocyclic rings may optionally be substituted with 1 to 3 substituents selected from the group consisting of hydroxy, halogen, nitro, cyano, amino, N-(C₁₋₆)alkylamino, N,N-di(C₁₋₆)alkylamino, (C₁₋₆)alkylthio, phenyl, phenoxy, benzyl, naphthyl, (C₁₋₆)alkyl optionally substituted by mono-, di- or tri-halogen, and (C₁₋₆)alkoxy optionally substituted by mono-, di- or tri-halogen,
      R¹⁰¹ represents one of the following carbocyclic or heterocyclic rings selected from the group consisting of cyclopropyl, cyclobutyl, cyclopentyl, cyclohexyl, pyrrolidinyl pyrrolyl, phenyl, pyridyl, pyrimidinyl, benzodioxolyl, naphthyl, indolyl, isoindolyl, quinolyl, isoquinolyl, and dihydroisoquinolyl,
         in any of which the carbocyclic or heterocyclic rings may optionally be substituted with 1 to 3 substituents selected from the group consisting of hydroxy, halogen, nitro, cyano, amino, N-(C₁₋₆alkyl)amino, N,N-di(C₁₋₆alkyl)amino, (C₁₋₆)alkylthio, phenyl, phenoxy, benzyl, naphthyl, (C₁₋₆)alkyl optionally substituted by mono-, di- or tri-halogen, and (C₁₋₆)alkoxy optionally substituted by mono-, di- or tri-halogen;
   R¹² represents one of the following carbocyclic or heterocyclic rings selected from the group consisting of cyclopropyl, cyclobutyl, cyclopentyl, cyclohexyl, pyrrolidinyl pyrrolyl, phenyl, pyridyl, pyrimidinyl, benzodioxolyl, naphthyl, indolyl, isoindolyl, quinolyl, isoquinolyl, and dihydroisoquinolyl,
      in any of which the carbocyclic or heterocyclic rings may optionally be substituted with 1 to 3 substituents selected from the group consisting of hydroxy, halogen, nitro, cyano, amino, N-(C₁₋₆)alkylamino, N,N-di(C)alkylamino, (C₁₋₆)alkylthio, phenyl, phenoxy, benzyl, naphthyl, (C₁₋₆)alkyl optionally substituted by mono-, di- or tri-halogen, and (C₁₋₆)alkoxy optionally substituted by mono-, di- or tri-halogen;
R² represents hydrogen, hydroxy, (C₂₋₆)alkenyl, (C₂₋₆)alkynyl, (C₃₋₇)cycloalkyl, pyrimidinyl, indolyl, pyridyl,
   (C₁₋₆)alkoxy optionally substituted by amino, N-(C₁₋₆)alkylamino, N,N-di(C₁₋₆)alkylamino or phenyl,
   (C₁₋₆)alkyl optionally substituted by phenyl, mono-, di- or tri-halogen, (C₁₋₆) alkylthio or (C₁₋₆) alkylsulfonyl,
   phenyl optionally substituted by halogen, hydroxy, nitro, ammo, N-(C₁₋₆)alkylamino, N-(dihydroimidazolyl)amino, (C₁₋₆)alkyl, (C₁₋₆)alkoxy optionally substituted by R²¹
      wherein
      R²¹ represents amino, N-(C₁₋₆)alkylamino, N,N-di(C₁₋₆)alkylamino or morpholino;
R³ represents hydrogen or (C₁₋₆)alkyl optionally substituted by mono-, di- or tri-halogen;
R⁴ represents carboxy, tetrazolyl or N-(hydroxy)aminocarbonyl;
R⁵ represents hydrogen, (C₁₋₆)alkyl, (C₁₋₆)alkoxy, phenyl or pyridinyl;
R⁶ represents hydrogen; and
R⁷ represents hydrogen, methyl or ethyl.

Another embodiment of the compounds of formula (I) is those wherein:
Ar represents 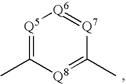
   Q⁵ and Q⁷ represent N;
   Q⁶ and Q⁸ independently represent CH or CR⁸,
      wherein
      R⁸ represents fluoro, chloro, amino, nitro, formyl, hydroxymethyl, trifluoromethyl, or methylthio;
Q¹, Q², Q³ and Q⁴ represent CH or CR¹⁰,
   wherein
   R¹⁰ represents halogen, amino, nitro, formyl, trifluoromethyl, hydroxymethyl, methylthio or benzyloxy;
R¹ represents —OR¹¹, —CH₂NHR¹¹, —C(O)R¹¹, —C(O)NHR¹¹, —SR¹¹, —SOR¹¹, —SO₂R¹¹, —NHR¹¹, —NHC(O)R¹¹, —NHC(O)OR¹¹, —NHC(O)NR¹¹, —NHSO₂R¹¹, hydrogen, hydroxy, halogen, benzodioxolyl, naphthyl,
   phenyl optionally substituted with 1 to 3 substituents selected from the group consisting of nitro, (C₁₋₆)alkoxy, (C₁₋₆)alkylthio, phenyl, and phenoxy,
   (C₁₋₆)alkyl optionally substituted by anilino, N-(benzyl)amino, indolyl, isoindolyl, quinolyl, isoquinolyl, dihydroisoquinolyl phenoxyimino, phenyl optionally substituted by halogen, or (C₁₋₆)alkoxy,
      wherein
      said (C₁₋₆)alkoxy optionally substituted by phenyl, pyridyl, benzodioxolyl, naphthyl, indolyl, isoindolyl, quinolyl, isoquinolyl, or dihydroisoquinolyl,
   (C₂₋₆)alkenyl optionally substituted by phenyl,
   (C₂₋₆)alkynyl optionally substituted by phenyl,
      wherein
      R¹¹ represents (C₁₋₆) alkoxy(C₁₋₆)alkylene,
   (C₁₋₆) alkyl optionally substituted by R¹⁰¹,
   (C₂₋₆)alkenyl optionally substituted by R¹⁰¹,
   (C₂₋₆)alkynyl optionally substituted by R¹⁰¹,
   or one of the following carbocyclic or heterocyclic rings selected from the group consisting of cyclopropyl, cyclobutyl, cyclopentyl, cyclohexyl, pyrrolidinyl pyrrolyl, phenyl, pyridyl, pyrimidinyl, benzodioxolyl, naphthyl, indolyl, isoindolyl, quinolyl, and dihydroisoquinolyl,
   in any of which the carbocyclic or heterocyclic rings may optionally be substituted with 1 to 3 substituents selected from the group consisting of hydroxy, halogen, nitro, cyano, (C₁₋₆)alkylthio, phenyl, phenoxy, benzyl, naphthyl, (C₁₋₆)alkyl optionally substituted by mono-, di- or tri-halogen, or (C₁₋₆)alkoxy optionally substituted by mono-, di- or tri-halogen,
   R¹⁰¹ represents one of the following carbocyclic or heterocyclic rings selected from the group consisting of cyclopropyl, cyclobutyl cyclopentyl, cyclohexyl, pyrrolidinyl pyrrolyl, phenyl, pyridyl, pyrimidinyl, benzodioxolyl, naphthyl, indolyl, isoindolyl, quinolyl and dihydroisoquinolyl,
      in any of which the carbocyclic or heterocyclic rings may optionally be substituted with 1 to 3 substituents selected from the group consisting of hydroxy, halogen, nitro, cyano, (C₁₋₆)alkylthio, phenyl, phenoxy, benzyl, naphthyl, (C₁₋₆)alkyl optionally substituted by r, di- or tri-halogen, and (C₁₋₆)alkoxy optionally substituted by mono-, di- or tri-halogen,
R¹ represents hydrogen, hydroxy, (C₂₋₆)alkenyl, (C₂₋₆)alkyl, pyrimidinyl, indolyl, pyridyl,
   (C₁₋₆) alkoxy optionally substituted by phenyl,
   (C₁₋₆) alkyl optionally substituted by phenyl, methylthio, mono-, di- or tri-halogen, or (C₁₋₆) alkylsulfonyl,
   phenyl optionally substituted by halogen, hydroxy, nitro, amino, N-(dihydroimidazolyl)amino or (C₁₋₆) alkoxy,
      wherein
      said (C₁₋₆)alkoxy optionally substituted by amino, N-(C₁₋₆)alkylamino, N,N-di(C₁₋₆)alkylthio, or morpholino;
R³ represents hydrogen or (C₁₋₆)alkyl;
R⁴ represents carboxy, tetrazolyl or N-(hydroxy)aminocarbonyl;
R⁵ represents hydrogen, phenyl or pyridyl;
R⁶ represents hydrogen; and
R⁷ represents hydrogen.

Another embodiment of the compounds of formula (I) is those wherein:
Ar represents 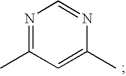
Q¹, Q², Q³ and Q⁴ represent CH;
R¹ represents hydrogen, hydroxy, halogen, benzodioxolyl, naphthyl, cyclopropylmethoxy, cyclobutylmethoxy, cyclopentylmethoxy, cyclohexylmethoxy, cyclopentylcarbonyl, cyclohexylcarbonyl, pyrrolidinylmethoxy, pyrrolidinylethoxy, phenoxy, benzyloxy, fluorobenzyloxy, difluorobenzyloxy, hydroxybenzyloxy, methoxybenzyloxy, dimethoxybenzyloxy, 1H-pyrrolylmethoxy, 1H-pyrrolylethoxy, pyridinyloxy, trifluorometylpyridinyloxy, pyridinylmethoxy, phenylethoxy, pyridinylethoxy, phenylpropoxy, cyanopyridinyloxy, pyrimidinyloxy, trifluoromethylpyrimidinyloxy, quinolinyloxy, benzoyl, fluorobenzoyl, chlorobenzoyl, anilinocarbonyl, benzylamino, benzoylamino, phenylacetylamino, phenylsulfonylamino, fuluoro phenylsulfonylamino, cyclopropylmethylamino, anilinomethyl,
   phenyl optionally substituted with 1 to 3 substituents selected from the group consisting of nitro, methoxy, ethoxy, methylthio, phenyl, and phenoxy,
   (C₁₋₆)alkyl optionally substituted by anilino, N-(benzyl)amino, indolyl, isoindolyl, quinolyl, isoquinolyl, dihydroisoquinolyl, phenoxy, phenoxyimino, or phenyl optionally substituted by halogen,
   (C₂₋₆)alkenyl optionally substituted by phenyl,
   (C₂₋₆)alkynyl optionally substituted by phenyl, or
   (C₁₋₆) alkoxy optionally substituted by trifluoro or methoxy;
R² represents hydrogen, (C₂₋₆)alkenyl, (C₂₋₆)alkynyl, pyrimidinyl, indolyl, pyridyl,
   (C₁₋₆)alkoxy optionally substituted by phenyl,
   (C₁₋₆)alkyl optionally substituted by phenyl, methylthio, mono-, di- or tri-halogen, or (C₁₋₆) alkylsulfonyl,
   phenyl optionally substituted by halogen, hydroxy, nitro, amino, N-(dihydroimidazolyl)amino or (C₁₋₆) alkoxy optionally substituted by amino, N-(C₁₋₆) alkylamino, N,N-di(C₁₋₆)alkylamino, or morpholino;
R³ represents hydrogen;
R⁴ represents carboxy or tetrazolyl;
R⁵ represents hydrogen;
R⁶ represents hydrogen; and
R⁷ represents hydrogen.

Preferably, said phenyl or heteroaryl amino alkane derivatives of the formula (I) is selected from the group consisting of:
3-(2-aminoethoxy)-N-{6-[4-(benzyloxy)phenyl]pyrimidin-4-yl}phenylalanine;
4-chloro-N-{6-[4-(cyclopropylmethoxy)phenyl]pyrimidin-4-yl}phenylalanine;
N-(6-{4-[(2-fluorobenzyl)oxy]phenyl}pyrimidin-4-yl)phenylalanine;
N-(6-{4-[(3,5-difluorobenzyl)oxy]phenyl}pyrimidin-4-yl)-3-pyridin-2-yl-alanine;
N-(6-{4-[(3,5-difluorobenzyl)oxy]phenyl}pyrimidin-4-yl)norleucine;
N-(6-{4-[(3,5-difluorobenzyl)oxy]phenyl}pyrimidin-4-yl)phenylalanine;
N-(6-{4-[(3,5-dimethoxybenzyl)oxy]phenyl}pyrimidin-4-yl)-3-pyridin-2-yl-alanine;
N-(6-{4-[(3,5-dimethoxybenzyl)oxy]phenyl}pyrimidin-4-yl)norleucine;
N-(6-{4-[(3,5-dimethoxybenzyl)oxy]phenyl}pyrimidin-4-yl)phenylalanine;
N-(6-{4-[(3-fluorobenzyl)oxy]phenyl}pyrimidin-4-yl)-3-pyridin-2-ylalanine;
N-(6-{4-[(3-fluorobenzyl)oxy]phenyl}pyrimidin-4-yl)phenylalanine;
N-(6-{4-[(3-methoxybenzyl)oxy]phenyl}pyrimidin-4-yl)-3-pyridin-2-ylalanine;
N-(6-{4-[(3-methoxybenzyl)oxy]phenyl}pyrimidin-4-yl)norleucine;
N-(6-{4-[(3-methoxybenzyl)oxy]phenyl}pyrimidin-4-yl)phenylalanine;
N-(6-{4-[(4-fluorobenzyl)oxy]phenyl}pyrimidin-4-yl)phenylalanine;
N-(6-{4-[2-(1H-pyrrol-1-yl)ethoxy]phenyl}pyrimidin-4-yl)phenylalanine;
N-[6-(3′-methoxybiphenyl-4-yl)pyrimidin-4-yl]phenylalanine;
N-[6-(4′-methoxybiphenyl-4-yl)pyrimidin-4-yl]phenylalanine;
N-{6-[4-(1,3-benzodioxol-5-yl)phenyl]pyrimidin-4-yl}phenylalanine;
N-{6-[4-(2-phenylethoxy)phenyl]pyrimidin-4-yl}-3-pyridin-2-yl-alanine;
N-{6-[4-(2-phenylethoxy)phenyl]pyrimidin-4-yl}phenylalanine;
N-{6-[4-(benzyloxy)-3-fluorophenyl]pyrimidin-4-yl}-3-pyridin-2-ylalanine;
N-{6-[4-(benzyloxy)-3-fluorophenyl]pyrimidin-4-yl}phenylalanine;
N-{6-[4-(benzyloxy)phenyl]-5-fluoropyrimidin-4-yl}phenylalanine;
N-{6-[4-(benzyloxy)phenyl]pyrimidin-4-yl}-3-(2-morpholin-4-ylethoxy)phenylalanine;
N-{6-[4-(benzyloxy)phenyl]pyrimidin-4-yl}-3-[2-(dimethylamino)ethoxy]-phenylalanine;
N-{6-[4-(benzyloxy)phenyl]pyrimidin-4-yl}-3-hydroxyphenylalanine;
N-{6-[4-(benzyloxy)phenyl]pyrimidin-4-yl}-3-pyridin-2-yl-alanine;
N-{6-[4-(benzyloxy)phenyl]pyrimidin-4-yl}-4-chlorophenylalanine;
N{6-[4-(benzyloxy)phenyl]pyrimidin-4-yl}4-fluorophenylalanine;
N-{6-[4-(benzyloxy)phenyl]pyrimidin-4-yl}-norleucine;
N-{6-[4-(benzyloxy)phenyl]pyrimidin-4-yl}-phenylalanine;
N-{6-[4-(benzyloxy)phenyl]pyrimidin-4-yl}tryptophan;
N-{6-[4-(benzyloxy)phenyl]pyrimidin-4-yl}tyrosine;
N-{6-[4-(cyclopropylmethoxy)phenyl]pyrimidin-4-yl}-4-fluorophenylalanine;
N-{6-[4-(cyclopropylmethoxy)phenyl]pyrimidin-4-yl}-phenylalanine;
N-{6-[4-(phenoxymethyl)phenyl]pyrimidin-4-yl}phenylalanine;
N-{6-[4-(phenylethynyl)phenyl]pyrimidin-4-yl}phenylalanine;
N-{6-[4-(pyridin-3-ylmethoxy)phenyl]pyrimidin-4-yl}phenylalanine; and
N-{6-[6-(benzyloxy)pyridin-3-yl]pyrimidin-4-yl}phenylalanine;

More preferably, said phenyl or heteroaryl amino alkane derivatives of the formula (I) is selected from the group consisting of:
N-{6-[4-(benzyloxy)phenyl]pyrimidin-4-yl}-3-pyridin-2-yl-D-alanine;
N-{6-[4-(benzyloxy)phenyl]pyrimidin-4-yl}-D-norleucine;
N-{6-[4-(benzyloxy)phenyl]pyrimidin-4-yl}-D-phenylalanine; and
N-{6-[4-(cyclopropylmethoxy)phenyl]pyrimidin-4-yl}-D-phenylalanine.

Further, the present invention provides a medicament, which includes one of the compounds, described above and optionally pharmaceutically acceptable excipients.

Alkyl per se and “alk” and “alkyl” in alkoxy, alkanoyl, alkylamino, alkylaminocarbonyl, alkylaminosulphonyl, alkylsulphonylamino, alkoxycarbonyl, alkoxycarbonylamino and alkanoylamino represent a linear or branched alkyl radical having generally 1 to 6, preferably 1 to 4 and particularly preferably 1 to 3 carbon atoms, representing illustratively and preferably methyl, ethyl, n-propyl, isopropyl, tert-butyl, n-pentyl and n-hexyl.

Alkoxy illustratively and preferably represents methoxy, ethoxy, n-propoxy, isopropoxy, tert-butoxy, n-pentoxy and n-hexoxy.

Alkylamino represents an alkylamino radical having one or two (independently selected) alkyl substituents, illustratively and preferably representing methylamino, ethylamino, n-propylamino, isopropylamino, tert-butylamino, n-pentylamino, n-hexyl-amino, N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

Cycloalkyl illustratively and preferably represent such as cyclopropyl, cyclobutyl, cyclopentyl, cyclohexyl or adamantyl.

Aryl per se or in combination with any other term, represents a mono- to tricyclic aromatic carbocyclic radical having generally 6 to 14 carbon atoms and more preferably from 6-10 carbon atoms. Examples of aryl radicals include, but are not limited to phenyl, naphthyl, indenyl indanyl, azulenyl, fluorenyl, anthracenyl, phenanthrenyl and the like.

Heteroaryl per se or in combination with any other term, represents an aromatic mono- or bicyclic radical having generally 5 to 10 and preferably 5 or 6 ring atoms and up to 5 and preferably up to 4 hetero atoms selected from the group consisting of S, O and N, illustratively and preferably representing thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl.

Heterocyclic ring represents a 3- to 15-membered ring radical which consists of carbon atoms and from one to five heteroatoms selected from the group consisting of nitrogen, oxygen and sulfur. The heterocyclic ring radical may be a monocyclic, bicyclic or tricyclic ring system, which may include fused or bridged ring systems; and the nitrogen, carbon or sulfur atoms in the heterocyclic ring radical may be optionally oxidized and the heterocyclic ring system may be partially or fully saturated or aromatic. Examples of such rings include, but are not limited to thienyl, furyl, benzothienyl, furanyl, benzofuranyl, pyrazinyl, pyrazolyl, pyridazinyl, pyridyl, pyrimidinyl, pyrrolyl, isothiazolyl, thiazolyl, oxazolyl, isoxazolyl, triazolyl, tetrazolyl, imidazolyl, thiadiazoyl, benzothiadiazolyl, oxadiazolyl, benzothiazolyl, indolyl, indazolyl, carbazolyl, quinolyl, isoquinolyl, benzodioxolyl, indazolyl, indazolinolyl, pyrrolidinyl, piperidinyl, pyranyl, pyrazolinyl, piperazinyl, morpholinyl, thiamorpholinyl, thiazolidinyl, benzofuranoyl, thiamorpholinyl sulfone, benzoxazolyl, oxopiperidinyl, oxopyrrolidinyl, oxoazopinyl, azepinyl, furazanyl, tetrahydropyranyl, tetrahydrofuranyl, dioxolyl, dioxinyl, oxathiolyl, benzodioxolyl and the like.

Aralkyl represents any alkyl group substituted with an aryl group in which, illustratively and preferably, the aryl and alkyl are as previously described. Examples of such aralkyl includes, but is not limited to, such as benzyl, phenethyl, naphtylmethyl, diphenylmethyl, and the like.

### EMBODIMENT OF THE INVENTION

The compound of the formula (I) of the present invention can be, but not limited to be, prepared by combining various known methods. In some embodiments, one or more of the substituents, such as amino group, carboxyl group, and hydroxyl group of the compounds used as starting materials or intermediates are advantageously protected by a protecting group known to those skilled in the art. Examples of the protecting groups are described in “Protective Groups in Organic Synthesis (3rd Edition)” by Greene and Wuts, John Wiley and Sons, New York 1999.

The compound of the formula (I) of the present invention can be, but not limited to be, prepared by the Method [A] or [B] below.

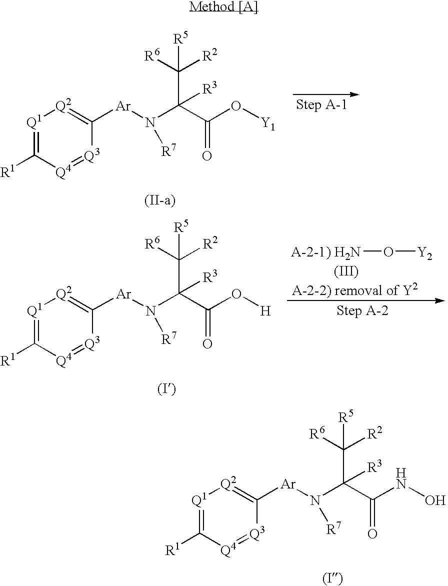

In the Step A-1, the compound of the formula (I) (wherein Ar, Q¹, Q², Q³, Q⁴, R¹, R², R³, R⁵, R⁶ and R⁷ are the same as defined above) can be obtained by the hydrolysis of the compound of formula (II-a) (wherein Ar, Q¹, Q², Q³, Q⁴, R¹, R², R³ R⁵, R⁶ and R⁷ are the same as defined above, and Y₁ represents C₁₋₆ alkyl).

The reaction can be advantageously carried out in the presence of a base including, for instance, alkali metal hydroxide such as sodium hydroxide, lithium hydroxide potassium hydroxide; and the like.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; amides such as N,N-dimethylformamide (DMF), N,N-dimethylacetamide and N-methylpyrrolidone; sulfoxides such as dimethylsulfoxide (DMSO); alcohols such as methanol, ethanol, 1-propanol, isopropanol and tert-butanol; water, and the like. Optionally, two or more of the solvents selected from the listed above can be mixed and used.

The reaction temperature can be optionally set depending on the compounds to be reacted. The reaction temperature is usually, but not limited to, about 20° C. to 100° C. The reaction may be conducted for, usually, 30 minutes to 48 hours and preferably 1 to 24 hours.

In the Step A-2, the compound of the formula (I″) (wherein Ar, Q¹, Q², Q³, Q⁴, R¹, R², R³, R⁵, R⁶ and R⁷ are the same as defined above) can be obtained by reaction of the compound of the formula (I′) (wherein Ar, Q¹, Q², Q³, Q⁴, R¹, R², R³, R⁵, R⁶ and R⁷ are the same as defined above) with the compound (III) (wherein Y₂ represents a protecting group such as, but not limited to, tert-butyldimethylsilyl, trimethylsilyl, phenyl dimethylsilyl and the like) in two steps (A-2-1 and A-2-2).

In the Step A-2-1, the reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; nitrites such as acetonitrile; amides such as N,N-dimethylformamide (DMF), N,N-and dimethylacetamide (DMAC), and N-methylpyrrolidone (NMP); urea such as 1,3-dimethyl-2-imidazolidinone (DMI); sulfoxides such as dimethylsulfoxide (DMSO); and the like. Optionally, two or more of the solvents selected from the listed above can be mixed and used.

The reaction temperature can be optionally set depending on the compounds to be reacted. The reaction temperature is usually, but not limited to, about 0° C. to 200° C. and preferably about 10° C. to 100° C. The reaction may be conducted for, usually, 10 minutes to 48 hours and preferably 30 minutes to 24 hours.

The reaction can be advantageously carried out using coupling agent including, for instance, carbodiimides such as N,N-dicyclohexylcarbodiimide and 1-(3-dimethylaminopropyl)-3-ethylcarbodiimide, 1-hydroxybenzotiazole monohydrate (HOBt), benzotriazole-1-yl-oxy-tris-pyrrolidino-phosphonium hexafluorophosphate (PyBOP), and the like.

In the Step A-2-2, the removal of protecting group Y₂ can be conducted by using a tetrabutylammonium fluoride or trifluoroacetic acid in inert solvent, including, for instance, ethers such as diethyl ether, isopropyl ether, dioxane and tetrahydrofuran (THF) and 1,2-dimethoxyethane; aromatic hydrocarbons such as benzene, toluene and xylene; dimethylformamide (DMF), and dimethylacetamide(DMAC).

The reaction temperature is usually, but not limited to, about 0° C. to 200° C. and preferably about 20° C. to 100° C. The reaction may be conducted for, usually, 30 minutes to 48 hours and preferably 2 hours to 24 hours.

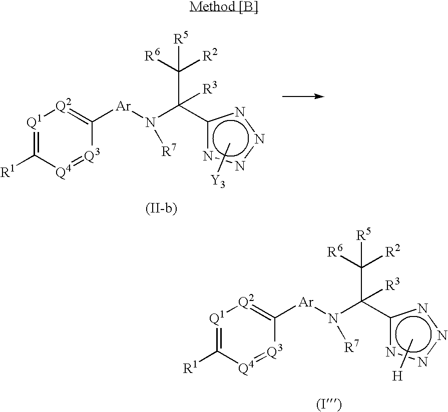

The compound of the formula (I″′) (wherein Ar, Q¹, Q², Q³, Q⁴, R¹, R², R³, R⁵, R⁶ and R⁷ are the same as defined above) can be obtained by the removal of Y₃ of the compound of formula (II-b) (wherein Ar, Q¹, Q², Q³, Q⁴, R¹, R², R³, R⁵, R⁶ and R⁷ are the same as defined above, and Y₃ represents a protecting group such as 2-(trimethylsillyl)ethoxymethyl (SEM), 2-methoxyethoxymethyl (MEM), triphenymethyl, and the like).

The removal of protecting group Y₃ can be conducted by using a reagent including, for instance, an acid such as trifluoroacetic acid and hydrochloric acid, or tetrabutylammonium fluoride.

The reaction may be carried out without solvent or in a solvent including, for instance, halogenated hydrocarbons such as dichloromethane, chloroform and 1,2-dichloroethane; alcohols such as methanol, ethanol, 1-propanol and isopropanol acetic acid, and the like. Optionally, two or more of the solvents selected from the listed above can be mixed and used.

The reaction temperature can be optionally set depending on compounds to be reacted. The reaction temperature is usually, but not limited to, about 20° C. to 120° C. The reaction may be conducted for, usually, 30 minutes to 60 hours and preferably 1 to 48 hours.

Preparation of the Compound of Intermediate

Method [C]

The compound of the formula (II) (wherein Ar, Q¹, Q², Q³, Q⁴, R¹, R², R³, R⁵, R⁶ and R⁷ are the same as defined above and R^{a} represents 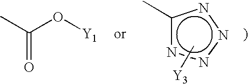
can be prepared by the following procedures;

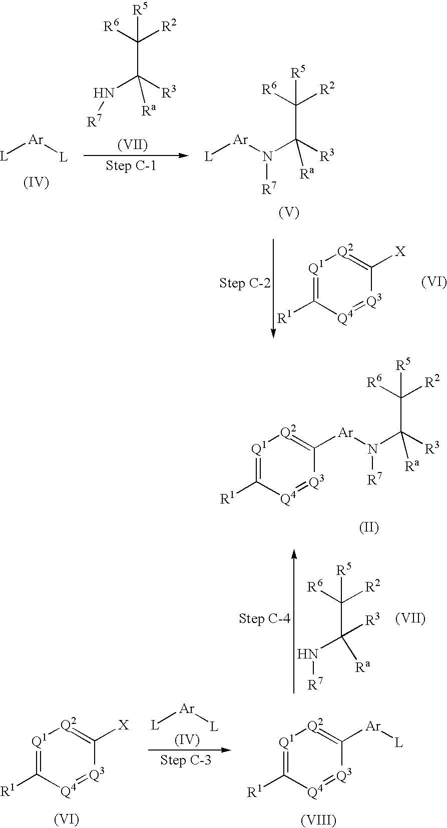

In the Step C-1, the compound of the formula (V) (wherein Ar, R^{a}, R², R³, R⁵, R⁶ and R⁷ are the same as defined above and L represents a leaving group including, for example, halogen atom such as chlorine, bromine, or iodine atom; and C₁₋₄alkylsulfonyloxy group, e.g., trifluoromethanesulfonyloxy, methanesulfonyloxy and the like) can be obtained by the reaction of the compound of the formula (IV) (wherein Ar and L are the same as defined) with the compound of the formula (VII) (wherein R^{a}, R², R³, R⁵, R⁶ and R⁷ are the same as defined above).

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; amides such as N,N-dimethylformamide (DMF), N,N-dimethylacetamide and N-methylpyrrolidone; sulfoxides such as dimethylsulfoxide (DMSO); alcohols such as methanol, ethanol, 1-propanol, isopropanol and tert-butanol and the like. Optionally, two or more of the solvents selected from the listed above can be mixed and used.

The reaction can be advantageously carried out in the presence of a base including, for instance, organic amines such as pyridine, triethylamine and N,N-diisopropylethylamine, dimethylaniline, diethylaniline, and the like.

The reaction can be advantageously carried out in the presence of a palladium catalyst such as tetrakis(triphenylphosphine)palladium.

The reaction temperature can be optionally set depending on the compounds to be reacted. The reaction temperature is usually, but not limited to, about 20° C. to 100° C. The reaction may be conducted for, usually, 30 minutes to 48 hours and preferably 1 to 24 hours.

In the Step C-2, the compound of the formula (II) (wherein Ar, Q¹, Q², Q³, Q⁴, R^{a}, R¹, R², R³, R⁵, R⁶ and R⁷ are the same as defined above) can be obtained by the reaction of the compound of the formula (V) (wherein L, Ar, R^{a}, R², R³, R⁵, R⁶ and R⁷ are the same as defined above) with the compound of the formula (VI) (wherein Q¹, Q², Q³, Q⁴ and R¹ are the same as defined above and X represents metal group including, for instance, organoborane group such as boronic acid and di-methoxy boryl; organostannyl group such as tributyl stannyl, and the like.) in the presence of a palladium catalyst such as tetrakis(triphenylphosphine)palladium.

The reaction can be advantageously carried out in the presence of a base including, for instance, cesium carbonate, sodium carbonate, potassium carbonate, and the like.

The reaction may be carried out in a solvent including, for instance, ethers such as diethyl ether, isopropyl ether, dioxane and tetrahydrofuran (THF) and 1,2-dimethoxyethane; aromatic hydrocarbons such as benzene, toluene and xylene; amides such as N,N-dimethylformamide (DMF), N,N-dimethylacetamide and N-methylpyrrolidone; sulfoxides such as dimethylsulfoxide (DMSO); alcohols such as methanol, ethanol, 1-propanol, isopropanol and tert-butanol and the like. Optionally, two or more of the solvents selected from the listed above can be mixed and used.

The reaction temperature can be optionally set depending on the compounds to be reacted. The reaction temperature is usually, but not limited to, about 20° C. to 120° C. The reaction may be conducted for, usually, 30 minutes to 48 hours and preferably 1 to 24 hours.

Alternatively, the compound of the formula (II) (wherein Ar, Q¹, Q², Q³, Q⁴, R^{a}, R¹, R², R³, R⁵, R⁶ and R⁷ are the same as defined above) can be obtained by the following procedures;

In the Step C-3, the compound of the formula (VIII) (wherein L, Ar, Q¹, Q², Q³, Q⁴, and R¹ are the same as defined above) can be obtained by the reaction of the compound of the formula (VI) (wherein Q¹, Q², Q³, Q⁴, R¹ and X are the same as defined above) with the compound of the formula (IV) (wherein L and Ar are the same as defined above) in a similar manner described in Step C-2 of Method [C] for the preparation of the compound of the formula (II).

In the Step C-4, the compound of the formula (II) (wherein Ar, Q¹, Q², Q³, Q⁴, R^{a}, R¹, R², R³, R⁵, R⁶ and R⁷ are the same as defined above) can be obtained by the reaction of the compound of the formula (VIII) (wherein L, Ar, Q¹, Q², Q³, Q⁴, and R¹ are the same as defined above) with the compound of the formula (VII) (wherein R^{a}, R², R³, R⁵, R⁶ and R⁷ are the same as defined above) in a similar manner described in Step C-1 of Method [C] for the preparation of the compound of the formula (V).

The compound of the formula (IV), (VI) and (VII) are commercially available or can be prepared by the use of known techniques.

Method [D]

The compound of the formula (II-i) (wherein Ar, Q¹, Q², Q³, Q⁴, R^{a}, R¹, R², R⁵ and R⁶ are the same as defined above) can be obtained by the following procedures;

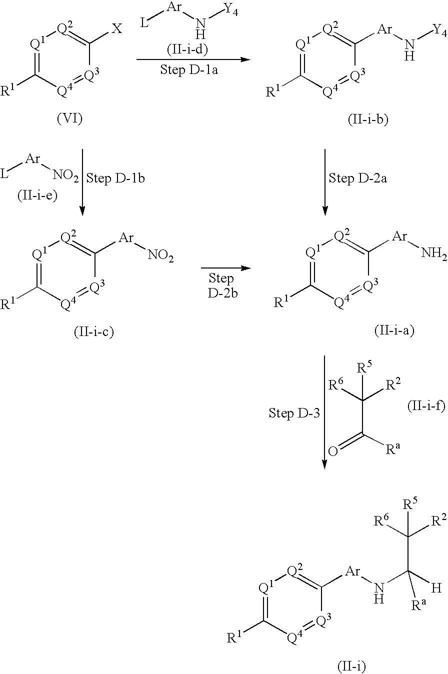

In the Step D-1a, the compound of the formula (II-i-b) (wherein Ar, Q¹, Q², Q³, Q⁴ and R¹ are the same as defined above and Y₄ represents a protecting group of amine including, for instance, tert-butoxycarbonyl, 9-fluorenylmethoxycarbonyl and the like) can be obtained by the reaction of the compound of the formula (VI) (wherein Q¹, Q², Q³, Q⁴, R¹ and X are the same as defined above) with the compound of the formula (II-i-d) (wherein Ar, L and Y₄ are the same as defined above) in a similar manner described in Step C-2 of Method [C] for the preparation of the compound of the formula (II).

In the Step D-2a, the compound of the formula (II-i-a) (wherein Ar, Q¹, Q², Q³, Q⁴ and R¹ are the same as defined above) can be obtained by the removal of a protecting group Y₄ of the compound of the formula (II-i-b) (wherein Ar, Q¹, Q², Q³, Q⁴, R¹ and Y₄ are the same as defined above).

The removal of protecting group Y₄ can be done by using a reagent including, for instance, an acid such as trifluoroacetic acid or hydrochloric acid, or a base such as morpholine, piperazine and the like.

The reaction may be carried out without solvent or in a solvent including, for instance, ethers such as diethyl ether, isopropyl ether, dioxane and tetrahydrofuran (THF) and 1,2-dimethoxyethane; aromatic hydrocarbons such as benzene, toluene and xylene; nitrites such as acetonitrile; amides such as N,N-dimethylformamide (DMF), N,N-dimethylacetamide (DMAC) and N-methylpyrrolidone (NMP); urea such as 1,3-dimethyl-2-imidazolidinone (DMI); and the like. Optionally, two or more of the solvents selected from the listed above can be mixed and used.

The reaction temperature can be optionally set depending on compounds to be reacted. The reaction temperature is usually, but not limited to, about 20° C. to 120° C. The reaction may be conducted for, usually, 30 minutes to 60 hours and preferably 1 to 48 hours.

Alternatively in the Step D-1b, the compound of the formula (II-i-c) (wherein Ar, Q¹, Q², Q³, Q⁴ and R¹ are the same as defined above) can be obtained by the reaction of the compound of the formula (VI) (wherein Q¹, Q², Q³, Q⁴, R¹ and X are the same as defined above) with the compound of the formula (II-i-e) (wherein Ar and L are the same as defined above) in a similar manner described in Step C-2 of Method [C] for the preparation of the compound of the formula (II).

In the Step D-2b, the compound of the formula (II-i-a) (wherein Ar, Q¹, Q², Q³, Q⁴ and R¹ are the same as defined above) can be obtained by the reduction of nitro group of compound of the formula (II-i-c) (wherein Ar, Q¹, Q², Q³, Q⁴ and R¹ are the same as defined above) using an agent including, for instance, metals such as zinc and iron in the presence of acid including, for instance, hydrochloric acid and acetic acid and stannous chloride, or by hydrogenation using a catalyst including, for instance, palladium on carbon and platinum on carbon.

The reaction can be carried out in a solvent including, for instance, ethers such as diethyl ether, isopropyl ether, dioxane, tetrahydrofuran (THF) and 1,2-dimethoxyethane, aromatic hydrocarbons such as benzene, toluene and xylene, alcohols such as methanol, ethanol, 1-propanol, isopropanol and tert-butanol, water and the like.

The reaction may be carried out, usually, at room temperature to 100° C. for 30 minutes to 12 hours.

In the Step D-3, the compound of the formula (II-i) (wherein Ar, Q¹, Q², Q³, Q⁴, R^{a}, R¹, R², R⁵ and R⁶ are the same as defined above) can be prepared by the reaction of the compound of the formula (II-i-a) (wherein Ar, Q¹, Q², Q³, Q⁴ and R¹ are the same as defined above) with the compound of the formula (II-i-f) (wherein R^{a}, R², R⁵ and R⁶ are the same as defined above) in the presence of a reducing agent, for instance, such as sodium triacetoxyborohydride, sodium cyanoborohydride, and the like.

The reaction may be carried out in a solvent including, for instance, ethers such as diethyl ether, isopropyl ether, dioxane and tetrahydrofuran (THF) and 1,2-dimethoxyethane; aromatic hydrocarbons such as benzene, toluene and xylene; amides such as N,N-dimethylformamide (DMF), N,N-dimethylacetamide and N-methylpyrrolidone; alcohols such as methanol, ethanol 1-propanol, isopropanol and tert-butanol; organic acid such as acetic acid; water and the like. Optionally, two or more of the solvents selected from the listed above can be mixed and used.

The reaction temperature can be optionally set depending on the compounds to be reacted. The reaction temperature is usually, but not limited to, about 20° C. to 100° C. The reaction maybe conducted for, usually, 30 minutes to 48 hours and preferably 1 to 24 hours.

The compound of the formula (II-i-d), (II-i-e) and (II-i-f) are commercially available or can be prepared by the use of known techniques.

Method [E]

The compound of the formula (II-ii) (wherein Ar, Q¹, Q², Q³, Q⁴, R^{a}, R², R³, R⁵, R⁶, R⁷ and R¹¹ are the same as defined above and Z represents O, S or NH) can be obtained by the following procedures;

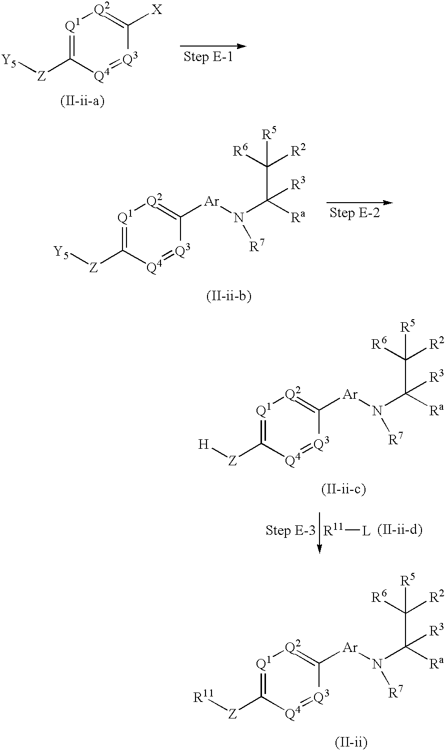

In the Step E-1, the compound of the formula (II-ii-b) (wherein Z, Ar, Q¹, Q², Q³, Q⁴, R^{a}, R², R³, R⁵, R⁶ and R⁷ are the same as defined above and Y₅ represents protecting groups such as oxygen-protecting group; for instance, C₁₋₆ alkyl, benzyl, 4-methoxybenzyl, 3,4-dimethoxybenzyl and the like, sulfur-protecting group; for instance, acetyl, benzoyl and the like, and amino-protecting group; for instance, t-butoxycarbonyl, 9-fluorenylmethoxycarbonyl and the like) can be obtained in a similar manner described in Method [C] or [D] for the preparation of the compound of the formula (II) or (II-i) by using the compound of the formula (II-ii-a) (wherein Z, Q¹, Q², Q³, Q⁴, X and Y₅ are the same as defined above) instead of the compound of the formula (VI).

In the Step E-2, the compound of the formula (II-ii-c) (wherein Z, Ar, Q¹, Q², Q³, Q⁴, R^{a}, R², R³, R⁵, R⁶ and R⁷ are the same as defined above) can be prepared by the removal of protecting group Y₅ of the compound of the formula (II-ii-b) (wherein Z, Ar, Q¹, Q², Q³, Q⁴, R^{a}, R², R³, R⁵, R⁶ and Y₅ are the same as defined above).

When Z refers to oxygen, the removal of protecting group Y₅ can be conducted by using a base including, for instance, sodium hydroxide, lithium hydroxide and potassium hydroxide, or an acid including, for instance, hydrochloric acid, trifluoroacetic acid and BBr₃. The deprotection can also be done by hydrogenation using a catalyst including, for instance, palladium on carbon and palladium hydroxide, when Y₅ is benzyl, 4-methoxybenzyl or 3,4-dimethoxybenzyl.

When Z refers to sulfur, the removal of protecting group Y₅ can be conducted by using a base such as sodium hydroxide, lithium hydroxide, potassium hydroxide, and the like.

When Z refers to amino, the removal of protecting group Y₅ can be conducted by using acids such as trifluoroacetic acid, hydrochloric acid, or base such as morpholine, piperazine and the like.

The reaction can be carried out in a solvent including, for instance, ethers such as diethyl ether, isopropyl ether, dioxane and tetrahydrofuran (THF) and 1,2-dimethoxyethane; aromatic hydrocarbons such as benzene, toluene and xylene; dimethylformamide (DMF), dimethylacetamide(DMAC), 1,3-dimethyl-3,4,5,6-tetrahydro-2(1H)-pyrimidinone (DMPU), 1,3-dimethyl-2-imidazolidinone (DMI), alcohols such as methanol, ethanol, 1-propanol, isopropanol and tert-butanol, water and the like. Optionally, two or more of the solvents selected from the listed above can be mixed and used.

The reaction temperature is usually, but not limited to, about 0° C. to 200° C. and preferably about 20° C. to 100° C. The reaction may be conducted for, usually, 30 minutes to 48 hours and preferably 2 hours to 24 hours.

In the Step E-3, the compound of the formula (II-ii) (wherein Z, Ar, Q¹, Q², Q³, Q⁴, R^{a}, R³, R⁵, R⁶, R⁷ and R¹¹ are the same as defined above) can be obtained by the reaction of the compound of the formula (II-ii-c) (wherein Z, Ar, Q¹, Q², Q³, Q⁴, R^{a}, R², R³, R⁵ and R⁷ are the same as defined above) with the compound of the formula (II-ii-d) (wherein R¹¹ and L are the same as defined above).

The reaction may be carried out in a solvent including, for instance, alcohols such as methanol and ethanol; ethers, such as dioxane, and tetrahydrofuran (THF); nitrites such as acetonitrile; amides such as dimethylformamide (DMF) and dimethylacetamide; sulfoxides such as dimethyl sulfoxide, and the like. Optionally, two or more of the solvents selected from the listed above can be mixed and used.

The reaction temperature of the reaction can be optionally set depending on the compounds to be reacted. The reaction temperature is usually, but not limited to, about −10° C. to 200° C. and preferably about 10° C. to 80° C. The reaction may be carried out for, usually, 30 minutes to 48 hours and preferably 1 to 24 hours.

The reaction can be advantageously conducted in the presence of a base. Examples of the base include an alkali metal hydride such as sodium hydride or potassium hydride; alkali metal alkoxide such as sodium methoxide or sodium ethoxide; alkali metal hydroxide such as sodium hydroxide or potassium hydroxide; carbonates such as sodium carbonate or potassium carbonate, and hydrogen carbonates such as sodium hydrogen carbonate and potassium hydrogen carbonate; organic amines such as triethylamine.

The compound of the formula (II-ii-a) and (II-ii-d) are commercially available or can be prepared by the use of known techniques.

Method [F]

The compound of formula (II-iii) (wherein Q¹, Q², Q³, Q⁴, R^{a}, R¹, R², R³, R⁵ and R⁶ are the same as defined above) can be, but not limited to be, obtained by the following procedures;

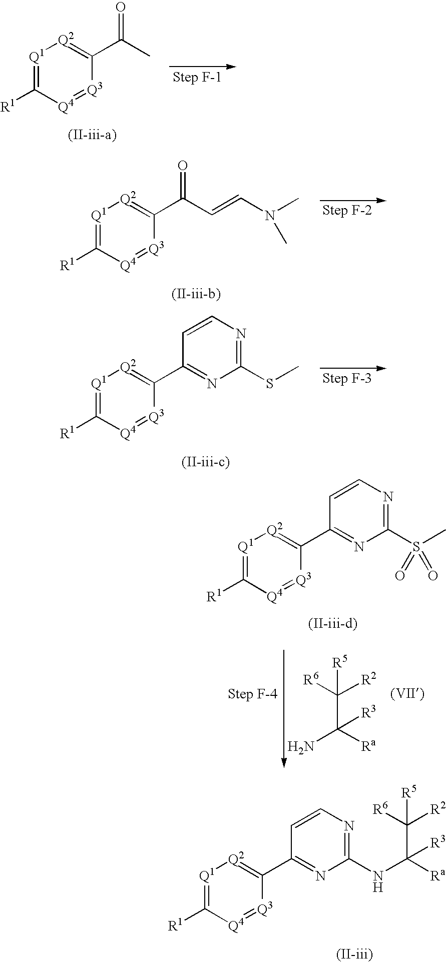

In the Step F-1, the compound of the formula (II-iii-b) (wherein Q¹, Q², Q³, Q⁴ and R¹ are the same as defined above) can be obtained by the reaction of the compound of formula (II-iii-a) (wherein Q¹, Q², Q³, Q⁴ and R¹ are the same as defined above) with N-[tert-butoxy(dimethylamino)methyl]-N,N-dimethylamine.

The reaction may be carried out in a solvent including, for instance; halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; amides such as N,N-dimethylformamide (DMF), N,N-dimethylacetamide and N-methylpyrrolidone; sulfoxides such as dimethylsulfoxide (DMSO); and the like. Optionally, two or more of the solvents selected from the listed above can be mixed and used.

The reaction temperature can be optionally set depending on the compounds to be reacted. The reaction temperature is usually, but not limited to, about 0° C. to 150° C.

The reaction may be conducted for, usually, 30 minutes to 48 hours and preferably 1 to 24 hours.

In the Step F-2, the compound of the formula (II-iii-c) (wherein Q¹, Q², Q³, Q⁴ and R¹ are the same as defined above) can be obtained by the reaction of the compound of formula (II-iii-b) (wherein Q¹, Q², Q³, Q⁴ and R¹ are the same as defined above) with thiourea and successive treatment with methyl iodide.

The reaction may be carried out in a solvent including, for instance, ethers such as diethyl ether, isopropyl ether, dioxane and tetrahydrofuran (THF) and 1,2-dimethoxyethane; aromatic hydrocarbons such as benzene, toluene and xylene; amides such as N,N-dimethylformamide (DMF), N,N-dimethylacetamide and N-methylpyrrolidone; sulfoxides such as dimethylsulfoxide (DMSO); alcohols such as methanol, ethanol, 1-propanol, isopropanol and tert-butanol and the like. Optionally, two or more of the solvents selected from the listed above can be mixed and used.

The reaction can be advantageously carried out in the presence of a base including, for instance, alkali metal hydroxide such as, sodium hydroxide, lithium hydroxide and potassium hydroxide; and the like.

The reaction temperature can be optionally set depending on the compounds to be reacted. The reaction temperature is usually, but not limited to, about 20° C. to 100° C. The reaction may be conducted for, usually, 30 minutes to 48 hours and preferably 1 to 24 hours.

In the Step F-3, the compound of the formula (II-iii-d) (wherein Q¹, Q², Q³, Q⁴ and R¹ are the same as defined above) can be obtained by the oxidation reaction of the compound of formula (II-iii-c) (wherein Q¹, Q², Q³, Q⁴ and R¹ are the same as defined above) using oxidating agent for instance, such as hydrogen peroxide, m-chloroperbenzoic acid, oxone, and the like.

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; alcohols such as methanol, ethanol 1-propanol, isopropanol and tert-butanol; water, and the like. Optionally, two or more of the solvents selected from the listed above can be mixed and used.

The reaction temperature can be optionally set depending on the compounds to be reacted. The reaction temperature is usually, but not limited to, about 0° C. to 150° C.

The reaction may be conducted for, usually, 30 minutes to 48 hours and preferably 1 to 24 hours.

The compound of the formula (II-iii-a) and (VII′) are commercially available or can be prepared by the use of known techniques.

Method [G]

The compound of formula (II-iv) (wherein Q¹, Q², Q³, Q⁴, R^{a}, R¹, R², R⁵ and R⁶ are the same as defined above and Ar′ represents 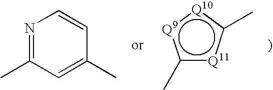
can be, but not limited to be, obtained by the following procedures;

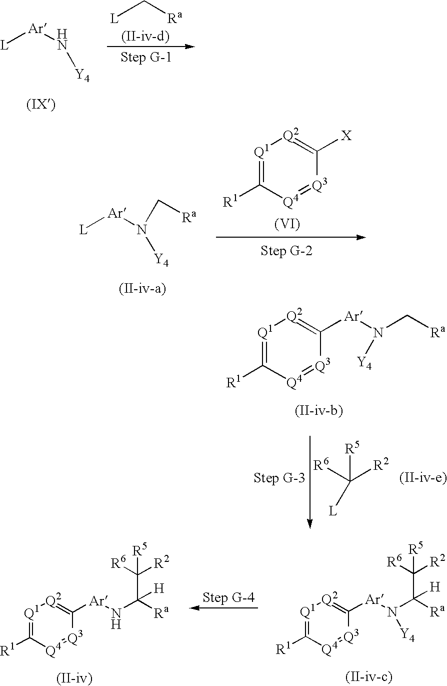

In the Step G-1, the compound of the formula (II-iv-a) (wherein Ar′, L, R^{a} and Y₄ are the same as defined above) can be obtained by the reaction of the compound of formula (IX′) (wherein Ar′, L and Y₄ are the same as defined above) with the compound of formula (II-iv-d) (wherein L and R^{a} are the same as defined above).

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; amides such as N,N-dimethylformamide (DMF), N,N-dimethylacetamide and N-methylpyrrolidone; sulfoxides such as dimethylsulfoxide (DMSO); alcohols such as methanol, ethanol, 1-propanol, isopropanol and tert-butanol and the like. Optionally, two or more of the solvents selected from the listed above can be mixed and used.

The reaction can be advantageously carried out in the presence of a base including, for instance, pyridine, sodium hydroxide or potassium carbonate and the like.

The reaction temperature can be optionally set depending on the compounds to be reacted. The reaction temperature is usually, but not limited to, about 20° C. to 100° C.

The reaction may be conducted for, usually, 30 minutes to 48 hours and preferably 1 to 24 hours.

In the Step G-2, the compound of the formula (11-iv-b) (wherein Ar′, Q¹, Q², Q³, Q⁴, R¹, R^{a} and Y₄ are the same as defined above) can be obtained by the reaction of the compound of the (II-iv-a) (wherein Ar′, L, R^{a} and Y₄ are the same as defined above) with the compound of the formula (VI) (wherein Q¹, Q², Q³, Q⁴, R¹ and X are the same as defined above) in a similar manner described in Step C-2 of Method [C] for the preparation of the compound of the formula (II).

In the Step G-3, the compound of the formula (II-iv-c) (Ar′, Q¹, Q², Q³, Q⁴, R^{a}, R¹, R², R⁵, R⁶ and Y₄ are the same as defined above) can be obtained by by the reaction of the compound of formula (I-iv-b) (wherein Ar′, Q¹, Q², Q³, Q⁴, R¹, R^{a} and Y₄ are the same as defined above) with the compound of formula (II-iv-e) (wherein L, R², R⁵ and R⁶ are the same as defined above).

The reaction may be carried out in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane; aromatic hydrocarbons such as benzene, toluene and xylene; amides such as N,N-dimethylformamide (DMF), N,N-dimethylacetamide, hexamethylphosphoric triamide, and N-methylpyrrolidone; sulfoxides such as dimethylsulfoxide (DMSO); alcohols such as methanol, ethanol, 1-propanol, isopropanol and tert-butanol and the like. Optionally, two or more of the solvents selected from the listed above can be mixed and used.

The reaction can be advantageously carried out in the presence of a base including, for instance, sodium hydride, lithium diisopropylamide, n-butyllithium, sodium bis(trimethylsilyl)amide and the like.

The reaction temperature can be optionally set depending on the compounds to be reacted. The reaction temperature is usually, but not limited to, about −100° C. to 50° C.

The reaction may be conducted for, usually, 30 minutes to 48 hours and preferably 1 to 24 hours.

In the Step G-4, the compound of the formula (II-iv) (wherein Ar′, Q¹, Q², Q³, Q⁴, R^{a}, R¹, R², R⁵ and R⁶ are the same as defined above) can be prepared by the removal of protecting group Y₄ of the compound of the formula (II-iv-c) (Ar′, Q¹, Q², Q³, Q⁴, R^{a}, R¹, R², R⁵, R⁶ and Y₄ are the same as defined above) in a similar manner described in Step D-2 a of Method [D] for the preparation of the compound of the formula (II-I a).

The compound of the formula (IX′), (II-iv-d) and (II-iv-e) are commercially available or can be prepared by the use of known techniques.

Method [H]

The compound of formula (II-v) (wherein Ar, Q¹, Q², Q³, Q⁴, R^{a}, R², R³, R⁵ and R⁶ are the same as defined above and R^{1′} represents carbocyclic ring, heterocyclic ring, C₁₋₆alkyl substituted by carbocyclic or heterocyclic ring, C₂₋₆alkenyl substituted by carbocyclic or heterocyclic ring, or C₂₋₆alkyl substituted by carbocyclic or heterocyclic ring) can be, but not limited to be, obtained by the following procedures;

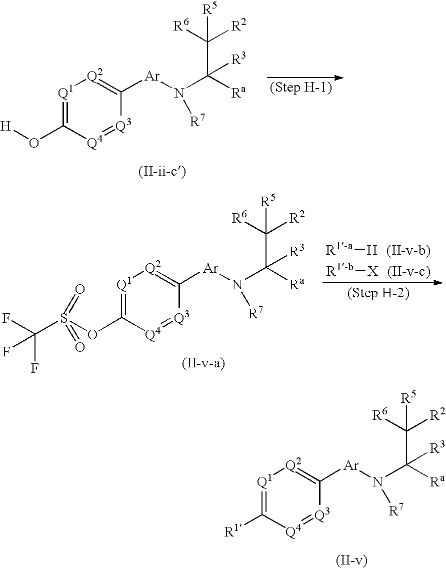

In the Step H-1, the compound of the formula (II-v-a) (wherein Ar, Q¹, Q², Q³, Q⁴, R^{a}, R², R³, R⁵, R⁶ and R⁷ are the same as defined above) can be obtained by conversion of the hydroxyl group of the compound (II-ii-c′) (wherein Ar, Q¹, Q², Q³, Q⁴, R^{a}, R², R³, R⁵, R⁶ and R⁷ are the same as defined above) by treatment with trifluoromethanesulfonic anhydride in a solvent including, for instance, halogenated hydrocarbons such as dichloromethane, chloroform and 1,2-dichloroethane; ethers such as diethyl ether, isopropyl ether, dioxane and tetrahydrofuran (THF) and 1,2-dimethoxyethane.

The reaction can be advantageously carried out in the presence of a base including, for instance triethylamine or pyridine and the like.

The reaction temperature can be optionally set depending on the compounds to be reacted. The reaction temperature is usually, but not limited to, about 0° C. to 100° C.

The reaction may be conducted for, usually, 30 minutes to 48 hours and preferably 1 to 24 hours.

In the Step H-2, the compound of the formula (II-v) (wherein Ar, Q¹, Q², Q³, Q⁴, R^{a}, R^{1′}, R², R³, R⁵, R⁶ and R⁷ are the same as, defined above) can be obtained by the compound of the formula (II-v-a) (wherein Ar, Q¹, Q², Q³, Q⁴, R^{a}, R², R³, R⁵, R⁶, and R⁷ are the same as defined) with the compound of the formula (II-v-b) (wherein R^{1′-a} represents heterocyclic rings substituted C₂₋₆alkenyl, or carbocyclic or heterocyclic rings substituted C₂₋₆alkyl) or the compound of the formula (II-v-c) (wherein R^{1′-b} carbocyclic or heterocyclic rings and X represents metal group including, for instance, organoborane group such as boronic acid and di-methoxy boryl; organostannyl group such as tributyl stannyl, and the like) in the presence of a palladium catalyst such as tetrakis(triphenylphosphine)palladium.

The reaction can be advantageously carried out in the presence of a base including, for instance, trimethylamine, triethylamine, cesium carbonate, sodium carbonate, potassium carbonate, and the like.

The reaction may be carried out in a solvent including, for instance, ethers such as diethyl ether, isopropyl ether, dioxane and tetrahydrofuran (THF) and 1,2-dimethoxyethane; aromatic hydrocarbons such as benzene, toluene and xylene; amides such as N,N-dimethylformamide (DMF), N,N-dimethylacetamide and N-methylpyrrolidone; sulfoxides such as dimethylsulfoxide (DMSO); alcohols such as methanol, ethanol 1-propanol, isopropanol and tert-butanol and the like. Optionally, two or more of the solvents selected from the listed above can be mixed and used.

The reaction temperature can be optionally set depending on the compounds to be reacted. The reaction temperature is usually, but not limited to, about 20° C. to 120° C. The reaction may be conducted for, usually, 30 minutes to 48 hours and preferably 1 to 24 hours.

The compound of the formula (II-v-b) and (II-v-c) are commercially available or can be prepared by the use of known techniques. The compound of the formula (II-ii-c′) can be prepared by Method [B].

The compounds of the formulas (II) including (II-i) to (II-v) can be further reacted to modify the substituents at R¹, R² and R¹⁰ of the formula (II) including (II-i) to (II-v) to synthesize the desired compounds in the scope of the present invention by the any conventional methods or combination of any conventional methods. Also, in the course of Method [A] to [H] above, the substituents at R¹, R² and R¹⁰ of the formula (II)including (II-i) to (II-v) can be modified.

When the compound shown by the formula (I) or a salt thereof has an asymmetric carbon in the structure, their optically active compounds and racemic mixtures are also included in the scope of the present invention.

Typical salts of the compound shown by the formula (I) include salts prepared by reaction of the compounds of the present invention with a mineral or organic acid, or an organic or inorganic base. Such salts are known as acid addition and base addition salts, successively.

Acids to form salts include inorganic acids such as, without limitation, sulfuric acid, phosphoric acid, hydrochloric acid, hydrobromic acid, hydriodic acid and the like, and organic acids, such as, without limitation, p-toluenesulfonic acid, methanesulfonic acid, oxalic acid, p-bromophenylsulfonic acid, succinic acid, citric acid, benzoic acid, acetic acid, and the like.

Base addition salts include those derived from inorganic bases, such as, without limitation, ammonium hydroxide, alkaline metal hydroxide, alkaline earth metal hydroxides, carbonates, bicarbonates, and the like, and organic bases, such as, without limitation, ethanolamine, triethylamine, tris(hydroxymethyl)aminomethane, and the like. Examples of inorganic bases include, sodium hydroxide, potassium hydroxide, potassium carbonate, sodium carbonate, sodium bicarbonate, potassium bicarbonate, calcium hydroxide, calcium carbonate, and the like.

The compound of the present invention or a salts thereof, depending on its substituents, may be modified to form lower alkylesters or known other esters; and/or hydrates or other solvates. Those esters, hydrates, and solvates are included in the scope of the present invention.

The compound of the present invention may be administered in oral forms, such as, without limitation normal and enteric coated tablets, capsules, pills, powders, granules, elixirs, tinctures, solution, suspensions, syrups, solid and liquid aerosols and emulsions.

They may also be administered in parenteral forms, such as, without limitation, intravenous, intraperitoneal, subcutaneous, intramuscular, and the like forms, well-known to those of ordinary skill in the pharmaceutical arts. The compounds of the present invention can be administered in intranasal form via topical use of suitable intranasal vehicles, or via transdermal routes, using transdermal delivery systems well-known to those of ordinary skilled in the art.

The dosage regimen with the use of the compounds of the present invention is selected by one of ordinary skill in the arts, in view of a variety of factors, including, without limitation, age, weight, sex, and medical condition of the recipient, the severity of the condition to be treated, the route of administration, the level of metabolic and excretory function of the recipient, the dosage form employed, the particular compound and salt thereof employed.

The compounds of the present invention are preferably formulated prior to administration together with one or more pharmaceutically-acceptable excipients. Excipients are inert substances such as, without limitation carriers, diluents, flavoring agents, sweeteners, lubricants, solubilizers, suspending agents, binders, tablet disintegrating agents and encapsulating material.

Yet another embodiment of the present invention is pharmaceutical formulation comprising a compound of the invention and one or more pharmaceutically-acceptable excipients that are compatible with the other ingredients of the formulation and not deleterious to the recipient thereof. Pharmaceutical formulations of the invention are prepared by combining a therapeutically effective amount of the compounds of the invention together with one or more pharmaceutically-acceptable excipients. In making the compositions of the present invention, the active ingredient may be mixed with a diluent, or enclosed within a carrier, which may be in the form of a capsule, sachet, paper, or other container. The carrier may serve as a diluent, which may be solid, semi-solid, or liquid material which acts as a vehicle, or can be in the form of tablets, pills, powders, lozenges, elixirs, suspensions, emulsions, solutions, syrups, aerosols, ointments, containing, for example, up to 10% by weight of the active compound, soft and hard gelatin capsules, suppositories, sterile injectable solutions and sterile packaged powders.

For oral administration, the active ingredient may be combined with an oral, and non-toxic, pharmaceutically-acceptable carrier, such as, without limitation, lactose, starch, sucrose, glucose, sodium carbonate, mannitol, sorbitol, calcium carbonate, calcium phosphate, calcium sulfate; methyl cellulose, and the like; together with, optionally, disintegrating agents, such as, without limitation, maize, starch, methyl cellulose, agar bentonite, xanthan gum, alginic acid, and the like; and optionally, binding agents, for example, without limitation, gelatin, natural sugars, beta-lactose, corn sweeteners, natural and synthetic gums, acacia, tragacanth, sodium alginate, carboxymethylcellulose, polyethylene glycol, waxes, and the like; and, optionally, lubricating agents, for example, without limitation, magnesium stearate, sodium stearate, stearic acid, sodium oleate, sodium benzoate, sodium acetate, sodium chloride, talc, and the like.

In powder forms, the carrier may be a finely divided solid which is in admixture with the finely divided active ingredient. The active ingredient may be mixed with a carrier having binding properties in suitable proportions and compacted in the shape and size desired to produce tablets. The powders and tablets preferably contain from about 1 to about 99 weight percent of the active ingredient which is the novel composition of the present invention. Suitable solid carriers are magnesium carboxymethyl cellulose, low melting waxes, and cocoa butter.

Sterile liquid formulations include suspensions, emulsions, syrups and elixirs. The active ingredient can be dissolved or suspended in a pharmaceutically acceptable carrier, such as sterile water, sterile organic solvent, or a mixture of both sterile water and sterile organic solvent.

The active ingredient can also be dissolved in a suitable organic solvent, for example, aqueous propylene glycol. Other compositions can be made by dispersing the finely divided active ingredient in aqueous starch or sodium carboxymethyl cellulose solution or in suitable oil.

The formulation may be in unit dosage form, which is a physically discrete unit containing a unit dose, suitable for administration in human or other mammals. A unit dosage form can be a capsule or tablets, or a number of capsules or tablets. A “unit dose” is a predetermined quantity of the active compound of the present invention, calculated to produce the desired therapeutic effect, in association with one or more excipients. The quantity of active ingredient in a unit dose may be varied or adjusted from about 0.1 to about 1000 milligrams or more according to the particular treatment involved.

Typical oral dosages of the present invention, when used for the indicated effects, will range from about 0.01 mg/kg/day to about 100 mg/kg/day, preferably from 0.1 mg/kg/day to 30 mg/kg/day, and most preferably from about 0.5 mg/kg/day to about 10 mg/kg/day. In the case of parenteral administration, it has generally proven advantageous to administer quantities of about 0.001 to 100 mg/kg/day, preferably from 0.01 mg/kg/day to 1 mg/kg/day. The compounds of the present invention may be administered in a single daily dose, or the total daily dose may be administered in divided doses, two, three, or more times per day. Where delivery is via transdermal forms, of course, administration is continuous.

### EXAMPLES

The present invention will be described in detail below in the form of examples, but they should by no means be construed as defining the meets and bounds of the present invention.

In the examples below, all quantitative data, if not stated otherwise, relate to percentages by weight.

Melting points are uncorrected. Liquid Chromatography—Mass spectroscopy (LC-MS) data were recorded on a Micromass Platform LC with Shimadzu Phenomenex ODS column (4.6 mm×30 mm) flushing a mixture of acetonitrile-water (9:1 to 1:9) at 1 ml/min of the flow rate. Mass spectra were obtained using electrospray (ES) ionization techniques (micromass Platform LC). TLC was performed on a precoated silica gel plate (Merck silica gel 60 F-254). Silica gel (WAKO-gel C-200 (75-150 μm)) was used for all column chromatography separations. All chemicals were reagent grade and were purchased from Sigma-Aldrich, Wako pure chemical industries, Ltd., Great Britain, Tokyo kasei kogyo Co., Ltd., Japan, Nacalai tesque, Inc., Watanabe Chemical Ind. Ltd., Maybridge plc, Lancaster Synthesis Ltd., Great Britain, Merck KgaA, Germany, Kanto Chemical Co., Ltd. ¹H NMR spectra were recorded using either Bruker DRX-300 (300 MHz for ¹H) spectrometer or Brucker 500 UltraShieled™ (500 MHz for 1H). Chemical shifts are reported in parts per million (ppm) with tetramethylsilane (TMS) as an internal standard at zero ppm. Coupling constant (J) are given in hertz and the abbreviations s, d, t, q, m, and br refer to singlet, doblet, triplet, quartet, multiplet, and broad, respectively. The mass determinations were carried out by MAT95 (Finnigan MAT).

The effects of the present compounds were examined by the following assays and pharmacological tests.

[Measurement of the [³H]-iloprost Binding to HEL Cells] (Assay 1)

A human erythloleukemia cell line, HEL 92.1.7, was purchased from American Type Culture Correction and maintained in RPMI-1640 medium (Gibco BRL) supplemented with 10% fetal calf serum (FCS), 2 mM glutamine, 4.5 g/L glucose, 10 mM Hepes, 1 mM sodium pyruvate, 100 U/ml penicillin, and 100 μg/ml streptomycin in a humidified 5% CO₂ atmosphere at 37° C. Cells were collected with centrifugation and washed with binding assay buffer (BAB: 50 mM Tris-HCl, 5 mM MgCl₂ (pH 7.5)). Cells were suspended at the density of 6.25×10⁶ cells/ml in BAB, and one million cells in 160 μl aliquot of cell suspension were put in a well of 96 well plate (Falcon). Then, 20 μl of compound solution, 100 μM of iloprost (for non-specific binding), or buffer alone (total binding), diluted with 1% DMSO in BAB was added. Finally, another 20 μl containing [³H]-iloprost (0.02 μCi, 0.5-1 pmol) in BAB was added and incubated at room temperature for 30 min with a gentle shaking. Cell suspension was then transferred to a well of MultiScreen plate with GF/C glass filters (Millipore) to harvest cells. Cells were washed twice with 200 μl of ice-cold BAB and the plate was kept at 55° C. for 30 min to dry filters. The filter in the well was punched out to a counting tube and 2 ml of Ultima Gold XR (Packard) was added. [³H]-radio activity in the filter was measured by a liquid scintillation counter (Beckman, USA).

[Iloprost-Induced cAMP Production Assay in HEL Cells] (Assay 2)

HEL cells were collected with centrifugation and washed with cAMP assay buffer (CAB: Hank's balanced salt solution, 17 mM Hepes, 0.1% bovine serum albumin, 1 mM IBMX, 0.4% DMSO, and 1 mM L-ascorbic acid sodium salt (pH 7.4)). Cells were suspended at the density of 2.5×10⁵ cells/ml in CAB, and twenty thousand cells in 80 μl aliquot of cell suspension were put in a well of 96 well plate (Falcon). Then, 10 μl of compound solution diluted with 1% DMSO in CAB or buffer alone was added. The plate was incubated at 37° C. for 30 min. Then, another 10 μl containing 100 nM iloprost in CAB or buffer alone was added and further incubated at 37° C. for 30 min. cAMP content in the well was measured by a cAMP ELISA kit (Applied Biosystems, USA).

[Measurement of Rhythmic Bladder Contraction in Anesthetized Rats]

(1) Animals
   Female Sprague-Dawley rats (200˜250 g/Charles River Japan) were used.
(2) Rhythmic bladder contraction in anesthetized rats

Rats were anesthetized by intraperitoneal administration of urethane (Sigma) at 1.25 g/kg. The trachea was cannulated with a polyethylene tube (HIBIKI, No. 8) to facilitate respiration; and a cannula (BECTON DICKINSON, PE-50) was placed in the left femoral vein for intravenous administration of testing compounds. The abdomen was opened through a midline incision, and after both ureters were cut, a water-filled balloon (about 1 ml capacity) was inserted through the apex of the bladder dome. The balloon was connected to a pressure transducer onto a polygraph. Rhythmic bladder contraction was elicited by raising up intravesical pressure to approximately 15 cm H₂O. After the rhythmic bladder contraction was stable, a testing compound was administered intravenously. Activity was estimated by measuring disappearance time and amplitude of the rhythmic bladder contraction. The effect on amplitude of bladder contractions was expressed as a percent suppression of the amplitude of those after the disappearance was recovered. Experimental values were expressed as the mean±S.E.M. The testing compounds-mediated inhibition of the rhythmic bladder contraction was evaluated using Student's t-test. A probability level less than 5% was accepted as significant difference.

Results of IP receptor binding/cAMP is shown in Examples and tables of the Examples below. The data corresponds to the compounds as yielded by solid phase synthesis and thus to levels of purity of about 40 to 90%. For practical reasons, the compounds are grouped in three classes of activity as follows:
IC₅₀=A<0.1 μM≦B<1 μM≦C

The compounds of the present invention also show excellent selectivity, and strong activity in vivo assays.

[Starting Compound 1A]

### 1-Iodo-4-cyclopropylmethoxybenzene

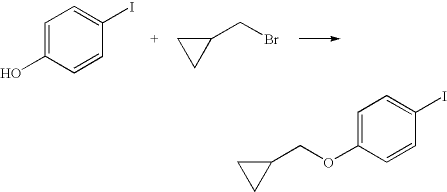

To a mixture of 4-iodophenol (108.6 g, 493.8 mmol), potassium carbonate (136.5 g, 988 mmol) and N,N-dimethylformamide (1 L) was added (bromomethyl)cyclopropane (72 mL, 741 mol), and the mixture was stirred at 80° C. for 4.5 hours. After cooling to room temperature, the resulting precipitates were filtered off and washed with ethyl acetate. The filtrate was concentrated under reduced pressure, and the resulting solid was recrystallized from methanol to give 1-iodo-4-cyclopropylmethoxy-benzene (124.8 g, 92%) as a colorless plate crystal.

### 4-(cyclopropylmethoxy)phenylboronic acid

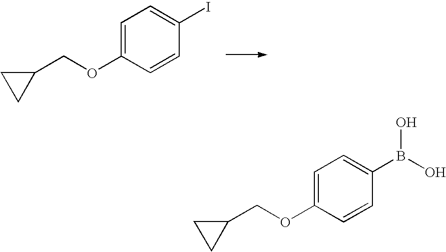

To a solution of 1-Iodo-4-cyclopropylmethoxy-benzene (1.9 g, 6.93 mmol) in tetra-hydrofuran (20 mL) at −78° C. was added dropwise n-butyl lithium (1.56 M in n-hexane, 5.33 mL, 8.32 mmol). After 20 minutes, trimethyl borate (1.2 mL, 10.4 mmol) was added dropwise. The reaction mixture was stirred for additional 30 minutes, and then allowed to warm to room temperature. The reaction was quenched with 1M hydrochloric acid (30 mL) and stirring was continued for 30 minutes. The mixture was extracted with diethyl ether and, the extracts were dried over magnesium sulfate and concentrated under reduced pressure. The residue was dissolved in toluene and then concentrated, and the resulting solid was washed with a mixture of hexane and ethyl acetate (8:2) to give 4-(cyclopropylmethoxy)phenylboronic acid (0.95 g, 71%) as a colorless solid.

[Starting Compound 1B]

### [4-(Anilinocarbonyl)phenyl]boronic acid

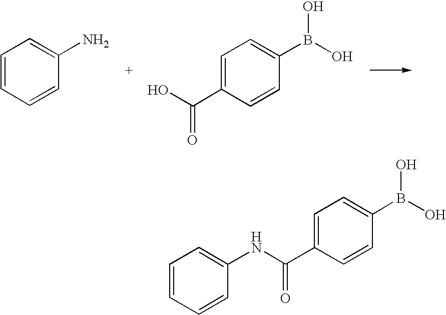

To a mixture of 4-carboxyphenyl boronic acid (0.200 g, 1.21 mmol), aniline (0.13 mL, 1.45 mmol) and triethylamine (0.34 mL, 2.41 mmol) in dichloromethane (3 mL) was added benzotriazole-1-yl-oxy-tris(pyrrolidine)-phosphonium hexafluorophosphate (0.753 g, 1.45 mmol) at room temperature, and the stirring was continued overnight. The mixture was diluted with water and extracted with ethyl acetate. The separated organic phase was washed with saturated sodium carbonate solution and brine, dried over sodium sulfate, filtered and concentrated under reduced pressure. The residue was purified by recrystallization from ethyl acetate to give [4-(anilinocarbonyl)phenyl]boronic acid (0.183 g, 63%) as a colorless solid.

[Starting Compound 1C]

### (2E)-3-(4-Bromophenyl)-1-phenylprop-2-en-1-one

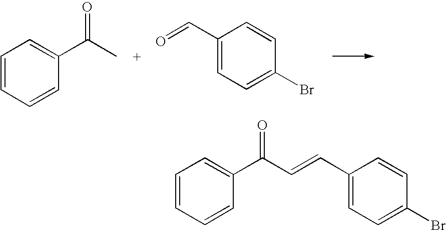

To a mixture of acetophenone (1.00 g, 8.32 mmol) and 4-bromobenzaldehyde (1.54 g, 8.32 mmol) and ethanol (15 mL) at 0° C. was added a solution of potassium hydroxide (1.03 g, 18.3 mmol) in water (10 mL). The reaction mixture was stirred for 1 hour at room temperature. The resulting precipitate was collected by filtration, washed with water and dried under reduced pressure to give (2E)-3-(4-bromophenyl)-1-phenylprop-2-en-1-one (2.10 g, 88%).

### 1-Bromo-4-(3-phenylpropyl)benzene

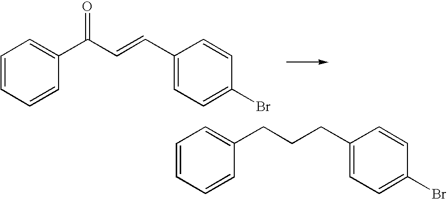

To a mixture of (2E)-3-(4-bromophenyl)-1-phenylprop-2-en-1-one (380 mg, 1.32 mmol) in trifluoroacetic acid (8 mL) at 0° C. was added dropwise triethylsilane (1.06 mL, 6.62 mmol). This mixture was stirred for 18 hours at room temperature. The reaction mixture was concentrated under reduced pressure. The residue was purified by column chromatography on silica gel (hexane/ethyl acetate 100:1) to give 1-bromo-4-(3-phenylpropyl)benzene (350 mg, 96%).

### [4-(3-Phenylpropyl)phenyl]boronic acid

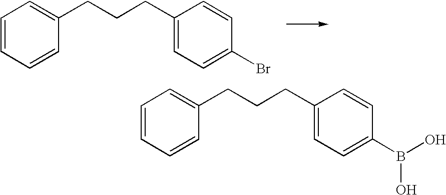

To a solution of 1-bromo-4-(3-phenylpropyl)benzene (350 mg, 1.27 mmol) in tetrahydrofuran (5 mL) at −78° C. was added n-butyllithium (1.53 M, 1.00 mL, 1.53 mmol in tetrahydrofuran). This mixture was stirred for 1 hour at −78° C., and then trimethyl borate (0.21 mL, 1.91 mmol) was added dropwise. The reaction mixture was stirred for 2 hours at −78° C., and then quenched with 1N hydrochloric acid. The mixture was stirred for 2 hours at room temperature and extracted with ethyl acetate. The organic phase was washed with water and brine, dried over magnesium sulfate, filtered, and concentrated under reduced pressure. The resulting precipitate was collected by filtration, washed with hexane, and dried under reduced pressure to give [4-(3-phenylpropyl)phenyl]boronic acid (120 mg, 39%).

### Example 1-1

### Methyl N-(6-chloropyrimidin-4-yl)-D-phenylalaninate

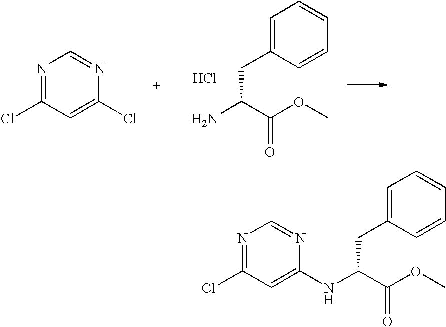

To a mixture of 4,6-dichloropyrimidine (57 g, 383 mmol), D-phenylalanine methyl ester hydrochloride (75 g, 348 mmol) and 1,4-dioxane (440 mL) was added N,N-diisopropylethylamine (123 mL, 730 mmol), and the mixture was stirred at 80° C. overnight. After cooled to room temperature, the mixture was concentrated under reduced pressure, and the residue was partitioned between ethyl acetate and water. The separated organic phase was washed with brine, dried over sodium sulfate, filtered and concentrated under reduced pressure. The crude product was purified by column chromatography on silica-gel (hexane:ethyl acetate, 3:1) to give methyl N-(6-chloropyrimidin-4-yl)-D-phenylalaninate (99.3 g, 98%) as a brown oil.

### Methyl N-{6-[4-(benzyloxy)phenyl] pyrimidin-4-yl}-D-phenylalaninate

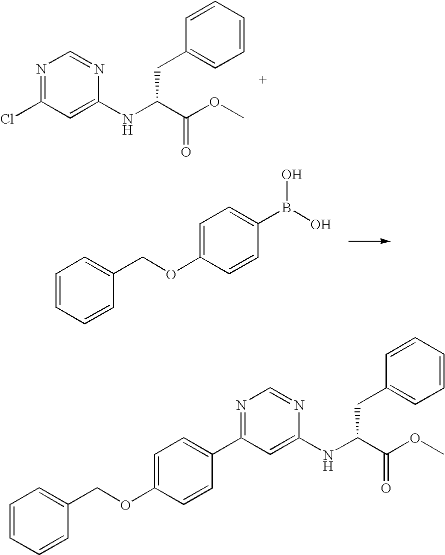

To a mixture of methyl N-(6-chloropyrimidin-4-yl)-D-phenylalaninate (30.0 g, 103 mmol), 4-benzyloxy)phenylboronic acid (28.1 g, 123 mmol), potassium carbonate (28.4 g, 206 mmol) and benzene (22 mL) under an argon atmosphere was added tetrakis(triphenylphosphine)palladium (5.94 g, 5.14 mmol). The mixture was stirred under reflux overnight. After cooled to room temperature, the mixture was diluted with ethyl acetate, and filtered through a Celite pad to remove inorganic salts. The filtrate was washed water and brine, dried over sodium sulfate, filtered, and concentrated under reduced pressure. The crude product was purified by column chromatography on silica-gel (hexane:ethyl acetate, 3:1-1:1). The product was triturated with diisopropyl ether (300 mL), and the suspension was stirred vigorously for 3 hours. The white precipitate was collected by filtration, washed with diisopropyl ether, and dried under reduced pressure to give methyl N-{6-[4-(benzyloxy)phenyl] pyrimidin-4-yl}-D-phenylalaninate (30.2 g, 67%) as a white solid.

### N-{6-[4-(Benzyloxy)phenyl] pyrimidin-4-yl}-D-phenylalanine

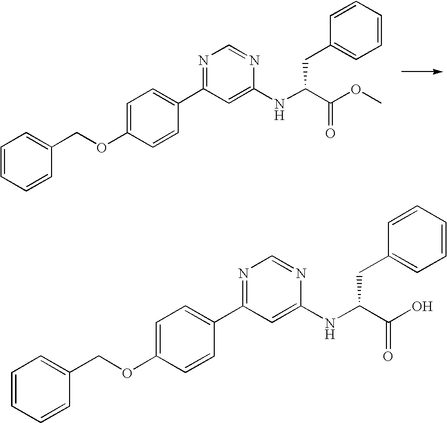

To a solution of methyl N-{6-[4-(benzyloxy)phenyl]-4-pyrimidinyl}-D-phenylalaninate (20.0 g, 45.5 mmol) in tetrahydrofuran (666 mL) at 0° C. was added dropwise 1N lithium hydroxide aqueous solution (90.0 mL, 90.0 mmol). The mixture was allowed to warm to room temperature, and stirring was continued for 2 hours. The mixture was neutralized at 0° C. by 1N HCl (90.0 mL, 90.0 mmol), then the mixture was concentrated under reduced pressure. The residue was partitioned between ethyl acetate and water. The separated organic phase was washed with brine, dried over sodium sulfate, filtered, and concentrated under reduced pressure. The crude product was purified by recrystallization from a mixture of acetonitrile and methanol to give methyl N-{6-[4-(benzyloxy)phenyl] pyrimidin-4-yl}-D-phenylalaninate (16.3 g, 84%) as a white solid.

Melting point: 150° C.

Molecular weight: 425.49

Mass spectrometry: 426 (M+H)⁺

In vitro activity grade: A

¹H-NMR (500 M, DMSO-d6): δ 3.00 (1H, dd, J=9.5, 13.9 Hz), 3.19 (1H, dd, J=4.6, 13.9 Hz), 4.77 (1H, br), 5.17 (2H, s), 6.98 (1H, br s), 7.11 (21, d, J=8.8 Hz), 7.18-7.20 (1H, m), 7.26-7.28 (4H, m), 7.32 (1H, t, J=7.4 Hz), 7.40 (2H, t, J=7.4 Hz), 7.47 (2H, d, J=7.4 Hz), 7.62 (1H, br), 7.93 (2H, d, J=8.0 Hz), 8.43 (1H, s), 12.74 (1H, br s).

Enantiomeric excess: >99% ee (DAICEL CHIRALCEL OJ, 0.1% phosphate buffer (pH 2): acetnitrile (65:35), flow rate; 1.0 mL/min, retention time; 7 min) Optical rotation: [α]_{D}+25° (c=1.0, DMF, 23° C.)

### Example 1-2

### Methyl N-{6-[4-(cyclopropylmethoxy)phenyl] pyrimidin-4-yl}-D-phenylalaninate

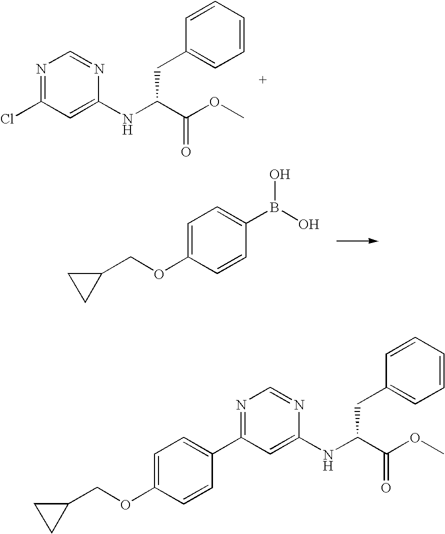

To a mixture of methyl N-(6-chloropyrimidin-4-yl)-D-phenylalaninate (1.27 g, 4.34 mmol), 4-(cyclopropylmethoxy)phenylboronic acid [starting compound 1A] (1.0 g, 5.21 mmol) and benzene (8.7 mL) under an argon atmosphere was added potassium carbonate (1.2 g, 8.68 mmol) followed by tetrakis(triphenylphosphine)palladium (0.25 g, 0.22 mmol). The mixture was stirred at reflux overnight. After cooled to room temperature, the mixture was filtered through a pad of celite and the filterate was partitioned between ethyl acetate and water. The separated organic phase was washed with brine, dried over sodium sulfate, filtered and concentrated under reduced pressure. The crude product was purified by column chromatography on silica-gel (hexane:ethyl acetate, 8:2) to give methyl N-{6-[4-(cyclopropylmethoxy)phenyl] pyrimidin-4-yl}-D-phenylalaninate (1.05 g, 60%) as a pale yelow oil.

### N-{6-[4-(Cyclopropylmethoxy)phenyl] pyrimidin-4-yl}-D-phenylalanine

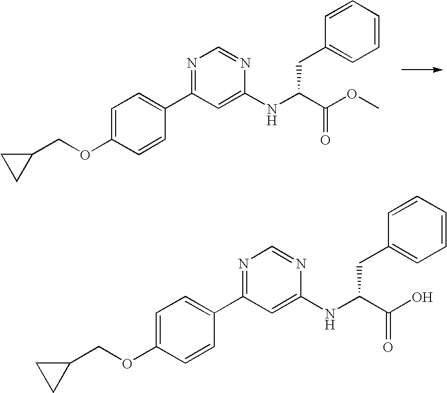

To a solution of methyl N-{6-[4-(cyclopropylmethoxy)phenyl] pyrimidin-4-yl}-D-phenylalaninate (5.0 g, 12 mmol) in THF (100 mL) at 0° C. was added dropwise 1M Lithium hydroxyde aqueous solution (24.8 mL, 24.8 mmol). The mixture was stirred at room temperature for 50 minutes, and diluted with water. The solution was washed with diethyl ether, and the separated aqueous phase was neutralized at 0° C. by 1M HCl (25 mL). The resulting precipitates were collected by filtration, and recrystallized from a mixture of acetnitrile and methanol to give N-{6-[4-(cyclopropylmethoxy)phenyl] pyrimidin-4-yl}-D-phenylalanine (4.1 g, 85%)

Melting point: 180-183° C. (dec)

Molecular weight: 389.453

Mass spectrometry: 390 (M+H)⁺

In vitro activity grade: A

¹H-NMR (500 MHz, MeOH-d4): δ 0.36(2H, ddd, J=4.4, 4.7, 6.0 Hz), 0.63 (2H, ddd, J=4.4, 6.0, 8.2 Hz), 1.27 (1H, m), 3.08 (1H, dd, J=8.5, 13.9 Hz), 3.89 (1H, dd, J=5.0, 13.9 Hz), 3.89 (2H, d, J=6.9 Hz), 4.96 (1H, br s), 6.85 (1H, br s), 7.01 (2H, d, J=8.8 Hz), 7.17 (1H, m), 7.26 (4H, m), 7.79 (2H, d, J=8.8 Hz), 8.40 (1H, s)

Enantiomeric excess: >99% ee (DAICEL, CHIRALCEL OJ 0.1% phosphate buffer (pH 2): acetnitrile (3:1), flow rate; 0.7 mL/min, retention time; 17 min)

Optical rotation: [α]_{D}=+29° (c=1.0, DMF, 23° C.)

### Example 1-3

### Ethyl D-norleucinate hydrochloride

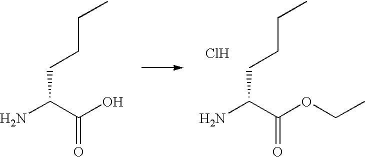

A solution of D-norleucine (15.0 g, 114 mmol) in ethanol (300 mL) was cooled to −70° C., and thionyl chloride (25.0 mL, 343 mmol) was added dropwise over 30 minutes. The mixture was heated under reflux overnight. After cooled to room temperature, the mixture was concentrated under reduced pressure to give ethyl D-norleucinate hydrochloride (22.2 g, quant.) as a colorless solid.

### Ethyl N-(6-chloropyrimidin-4-yl)-D-norleucinate

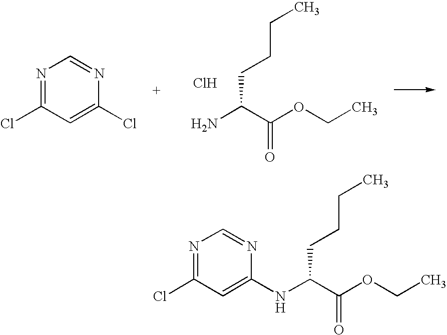

To a mixture of 4,6-dichloropyrimidine (15.0 g, 101 mmol) and ethyl D-norleucinate hydrochloride (21.7 g, 111 mmol) in dioxane (440 mL) was added dropwise N,N′-diisopropylethylamine (38.6 mL, 222 mmol). The mixture was stirred at 65° C. overnight, and then at 80° C. for 4 hours. After cooled to room temperature, the mixture was evaporated under reduced pressure. The residue was diluted with water, and the mixture was extracted with ethyl acetate. The separated organic layer was washed with brine, dried over sodium sulfate, filtered, and concentrated under reduced pressure. The resulting crude product was purified by column chromatography on silica-gel (hexane:ethyl acetate, 8:1-5:1-3:1) to give ethyl N-(6-chloropyrimidin-4-yl)-D-norleucinate (19.4 g, 71%) as a yellowish oil.

### Ethyl N-{6-[4-(benzyloxy) phenyl]pyrimidin-4-yl}-D-norleucinate

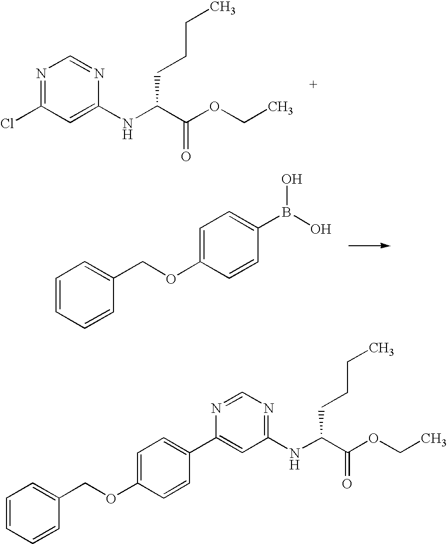

A mixture of ethyl N-(6-chloropyrimidin-4-yl)-D-norleucinate (19.0 g, 69.9 mmol), 4-(benzyloxy)phenylboronic acid (19.1 g, 83.9 mmol) and potassium carbonate (19.3 g, 140 mmol) in toluene (570 mL) was bubbled with argon gas for 10 minutes. Tetrakis(triphenylphosphine)palladium(0) (4.03 g, 3.50 mmol) was added to the mixture under argon gas, and the mixture was stirred at 80° C. for 20 hours. After cooled to room temperature, the mixture was partitioned between ethyl acetate and water. The organic layer was separated and washed with brine, dried over sodium sulfate, filtered and concentrated under reduced pressure. The residue was dissolved in chloroform (200 mL) and activated carbon (2 g) was added. The mixture was stirred for 2.5 hours. The mixture was passed through Celite and silica-gel pad with chloroform and the filtrate was concentrated. The resulting oil was purified by column chromatography on silica-gel (hexane:ethyl acetate, 5:1-3:1-1:1) to give ethyl N-{6-[4-(benzyloxy) phenyl]pyrimidin-4-yl}-D-norleucinate (22.38 g, 76%) as a yellowish solid.

### N-{6-[4-(benzyloxy) phenyl]pyrimidin-4-yl}-D-norleucine hydrochloride

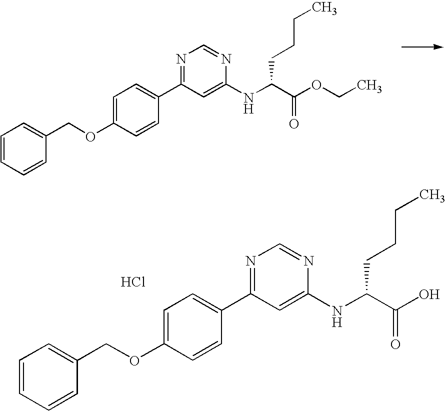

To a cold (0° C.) solution of N-{6-[benzyloxy)phenyl]pyrimidin-4-yl}-D-norleucinate (16.12 g, 38.42 mmol) in tetrahydrofuran (320 mL) was added 1N lithium hydroxide aqueous solution (76.9 mL, 76.9 mmol). The mixture was allowed to warm to room temperature, the stirring was continued for 6 hours. The mixture was concentrated under reduced pressure, and the residue was partitioned between diethyl ether and water. The separated aqueous phase was neutralized with 1N HCl (76.9 mL), and extracted twice with ethyl acetate. The combined organic phase was washed with brine, dried over sodium sulfate, filtered, and concentrated under reduced pressure.

The resulting yellowish solid was triturated with diisopropylethyl, and dried under reduced pressure to give a colorless solid. The product was dissolved in tetra-hydrofuran (300 mL), and treated with 4N hydrochloride in dioxane (9.6 mL). The resulting solid was collected by filtration, washed with tetrahydrofuran and diisopropyl ether, and then dried under reduced pressure. The solid obtained was purified by recrystallization from a mixture of tetrahydrofuran and water to give N{6-[4-(benzyloxy)phenyl]pyrimidin-4-yl}-D-norleucine hydrochloride (14.7 g, 89%) as a colorless solid.

Melting point: 199-203° C.

Molecular weight: 427.93

Mass spectrometry: 392 (M−HCl+H)⁺

In vitro activity grade: A

¹H-NMR (500 MHz, DMSO-d6): δ 0.88 (3H, t, J=7.2 Hz), 1.29-1.39 (4H, m), 1.79 (1H, br), 1.88 (1H, br), 4.62 (1H, br), 5.23 (2H, s), 7.09 (1H, br), 7.25 (2H, br), 7.35 (1H, t, J=7.3 Hz), 7.41 (2H, t, J=7.6 Hz), 7.48 (2H, d, J=7.3 Hz), 7.85 (2H, d, J=7.9 Hz), 8.75 (1H, br).

Enantiomeric excess: 98.7% ee (DAICEL, CHIRALCEL OJ 0.1% A phosphate buffer (pH 2): acetnitrile (65:35), flow rate; 1 mL/min, retention time; 6 min)

Optical rotation: [α]_{D}=+0.58° (c=1.0, DMF, 23° C.)

### Example 1-4

### Methyl 3-pyridin-2-yl-D-alaninate dihydrochloride

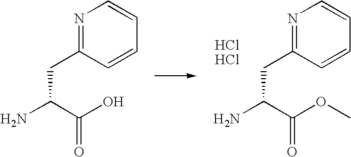

To a cooled (−40° C.) methanol (340 mL) was added dropwise thionyl chloride (65.8 mL, 903 mmol), and the mixture was gradually warmed up to room temperature. After 3-pyridin-2-yl-D-alanine (50.0 g, 301 mmol) was added portionwise, the resulting mixture was stirred at 80° C. for 5 hours. After cooled to room temperature, the reaction mixture was concentrated under reduced pressure. The residue was diluted with methanol and concentrated under reduced pressure. The residual solid was triturated with diethyl ether and dried at 60° C. under reduced pressure to give methyl 3-pyridin-2-yl-D-alaninate dihydrochloride (55.8 g, 73%) as a white powder.

### Methyl N-(6-chloropyrimidin-4-yl)-3-pyridin-2-yl-D-alaninate

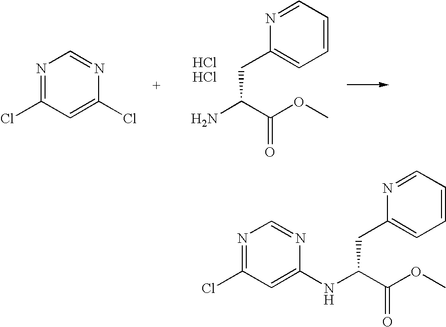

To a mixture of 4,6-dichloropyrimidine (28.1 g, 189 mmol), methyl 3-pyridin-2-yl-D-alaninate dihydrochloride (52.6 g, 208 mmol) and 1,4-dioxane (300 mL) was added N,N-diisopropylethylamine (102 mL, 586 mmol), and the mixture was stirred at 85° C. for 14 hours. After cooled to room temperature, the mixture was concentrated under reduced pressure, and the residue was partitioned between ethyl acetate and water. The separated organic phase was washed with water and brine, dried over magnesium sulfate, filtered, and concentrated under reduced pressure. The crude product was purified by column chromatography on silica-gel (hexane:ethyl acetate, 1:1) and washed with diisopropylether to give methyl N-(6-chloropyrimidin-4-yl)-3-pyridin-2-yl-D-alaninate (33.3 g, 60%) as a pale yellow solid.

### Methyl N-{6-[4-(benzyloxy)phenyl]pyrimidin-4-yl}-3-pyridin-2-yl-D-alaninate

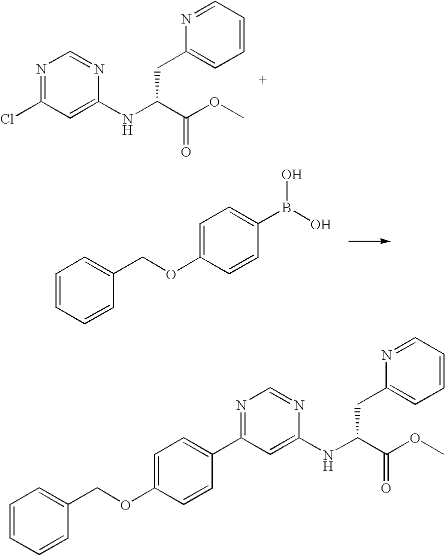

To a mixture of methyl N-(6-chloropyrimidin-4-yl)-3-pyridin-2-yl-D-alaninate (29.6 g, 101 mmol), 4-(Benzyloxy)phenylboronic acid (27.7 g, 121 mmol), potassium carbonate (27.9 g, 202 mmol) and benzene (60 mL) under an argon atmosphere was added tetrakis(triphenylphosphine)palladium (0) (5.00 g, 4.33 mmol). The mixture was stirred at 90° C. for 15 hours. After cooled to room temperature, the mixture was filtered through a Celite pad. The filtrate was concentrated under reduced pressure, and the residue was partitioned between ethyl acetate and water. The separated organic phase was washed with water and brine, dried over magnesium sulfate, filtered, and concentrated under reduced pressure. The crude product was purified by column chromatography on silica-gel (chloroform: ethyl acetate, 1:1) and washed with a mixture of diisopropyl ether and ethyl acetate (10:1) to give methyl N-{6-[4-(benzyloxy)phenyl]pyrimidin-4-yl}-3-pyridin-2-yl-D-alaninate (37.7 g, 85%) as a white solid.

Enantiomeric excess: >99% ee (DAICEL, CHIRALCEL OD hexane: ethanol (6:1), flow rate; 1 mL/min, retention time; 13 min).

### N-{6-[4-(benzyloxy)phenyl]pyrimidin-4-yl}-3-pyridin-2-yl-D-alanine

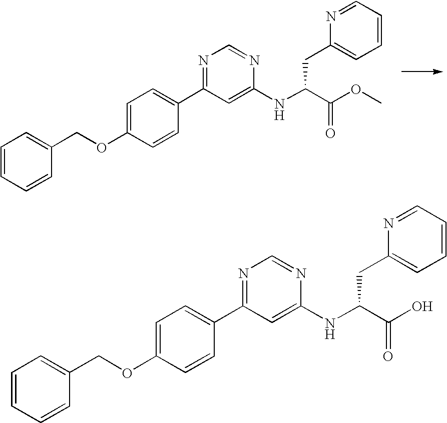

To a solution of methyl N-{6-[4-(benzyloxy)phenyl]pyrimidin-4-yl}-3-pyridin-2-yl-D-alaninate (30.8 g, 70.0 mmol) in methanol (100 mL) and tetrahydrofuran (400 mL) was added a solution of lithium hydroxide monohydrate (5.86 g, 140 mmol) in water (140 mL), and the mixture was stirred at room temperature for 3 hours. The mixture was neutralized at 0° C. with 1N hydrochloric acid solution. The volatile was removed under reduced pressure, and the precipitate was collected by filtration, washed with water, diisopropyl ether and methanol successively, and dried under reduced pressure to give N-{6-[4-(benzyloxy)phenyl]pyrimidin-4-yl}-3-pyridin-2-yl-D-alanine (21.9 g, 74%) as a white solid.

Melting point: 142° C.

Molecular weight: 426.47

Mass spectrometry: 427 (M+H)⁺

In vitro activity grade: A

¹H-NMR (500 MHz, DMSO-d6): δ 3.19 (1H, d, J=9.0, 12.7 Hz), 3.31 (1H, m), 5.01 (1H, br s), 5.17 (2H, s), 6.95 (1H, s), 7.11 (2H, d, J=9.0 Hz), 7.20-7.23 (1H, m), 7.31-7.35 (2H, m), 7.40 (2H, t, J=7.0 Hz), 7.47 (2H, t, J=7.3 Hz), 7.63 (1H, br), 7.70 (1H, dt, 1.9, 7.6 Hz), 7.93 (2H, d, J=7.9 Hz), 8.43 (1H, s), 8.49-8.51 (1H, m), 12.68 (1H, br s).

Enantiomeric excess: >99% ee. (The enantiomeric excess was determined by a chiral HPLC analysis of the corresponding methyl ester analog converted from the title product using diazomethane.)

Optical rotation: [α]_{D}=+33° (c>1.0, DMF, 23° C.).

### Examples 1-5 to 1-58

In the similar manners as described in Example 1-1 to Example 1-4 above, compounds in Examples 1-5 to 1-58 as shown in Table 1 were synthesized.

TABLE EXAMPLE 1

| Ex. | | | MASS | | |
|---|---|---|---|---|---|
| No. | Structure | M.W. | (M + 1) | MP | In vitro |
| 1-5 | 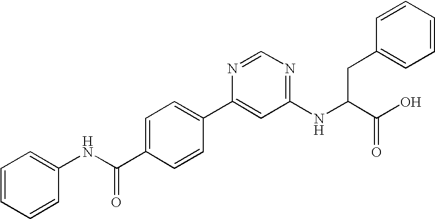 | 438.49 | 439 | 228-231Z | B |
| 1-6 | 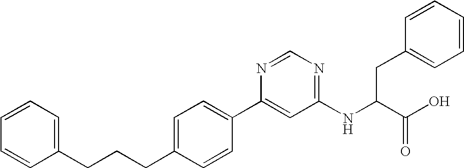 | 437.55 | 438 | 112-114 | A |
| 1-7 | 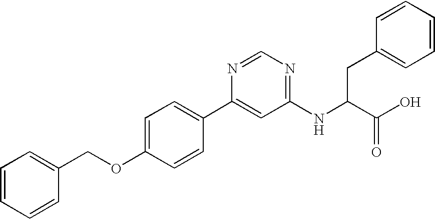 | 425.49 | 426 | 175-178 | A |
| 1-8 | 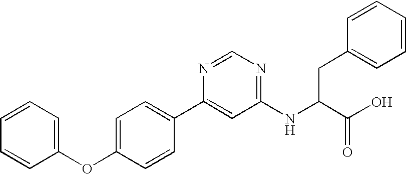 | 411.46 | 412 | 214-217Z | A |
| 1-9 | 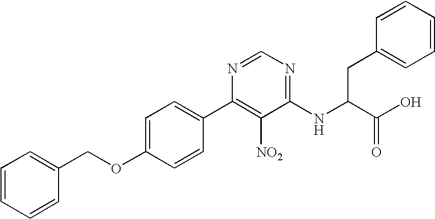 | 470.49 | 471 | <80 | C |
| 1-10 | 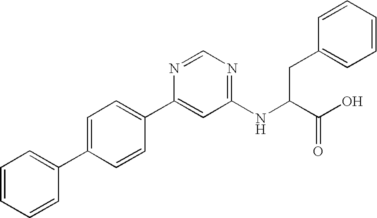 | 395.47 | 396 | 146-148Z | A |
| 1-11 | 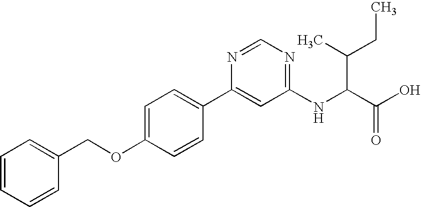 | 391.47 | 392 | 56 | B |
| 1-12 | 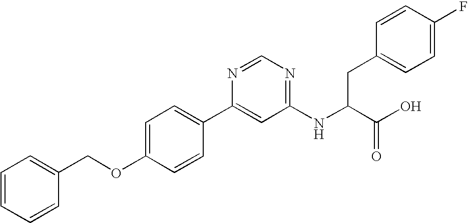 | 443.48 | 444 | 186 | A |
| 1-13 | 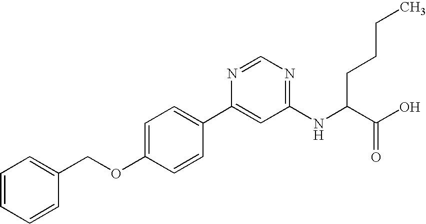 | 391.47 | 392 | 187 | A |
| 1-14 | 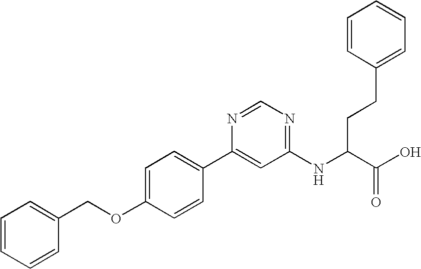 | 439.52 | 440 | 226-228 | A |
| 1-15 | 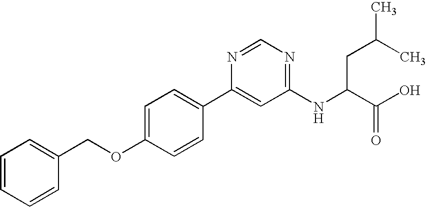 | 391.47 | 392 | 244-246 | B |
| 1-16 | 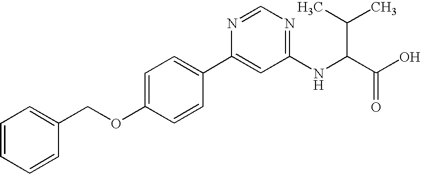 | 377.45 | 378 | 235-237 | B |
| 1-17 | 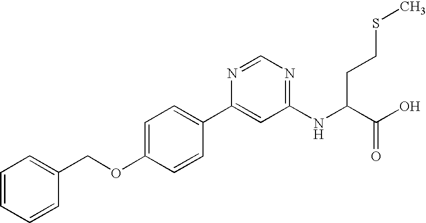 | 409.51 | 410 | 210 | A |
| 1-18 | 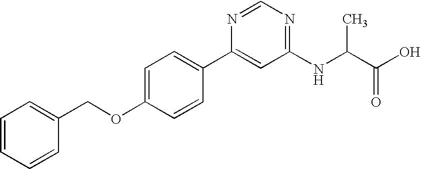 | 349.39 | 350 | 158 | B |
| 1-19 | 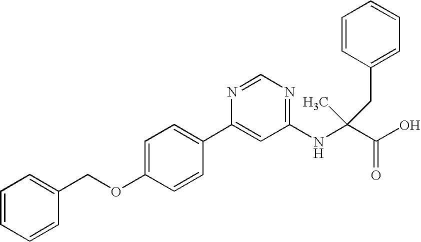 | 439.52 | 440 | 187 | A |
| 1-20 | 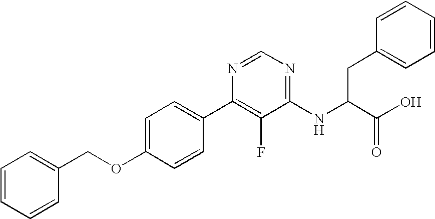 | 443.48 | 444 | 145-146 | A |
| 1-21 | 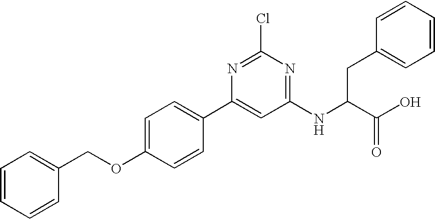 | 459.94 | 456 | 97-100 | A |
| 1-22 | 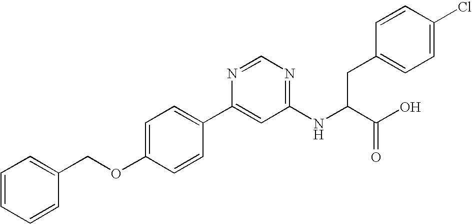 | 459.94 | 460 | 150-153Z | A |
| 1-23 | 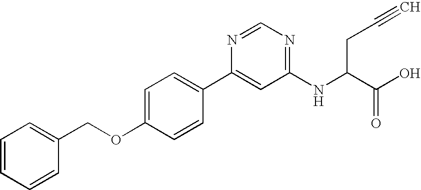 | 373.42 | 374 | 155 | A |
| 1-24 | 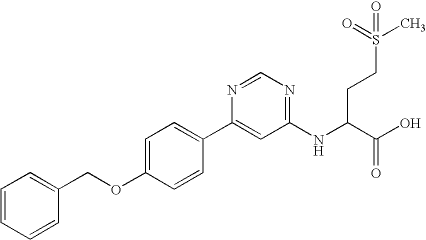 | 441.51 | 442 | 138 | B |
| 1-25 | 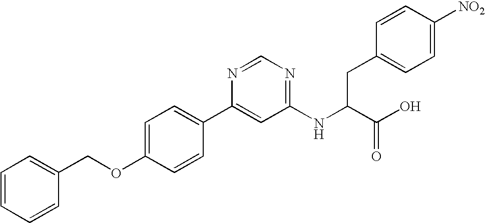 | 470.49 | 471 | 211 | A |
| 1-26 | 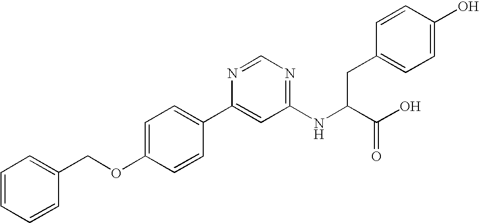 | 441.49 | 442 | 233-236 | A |
| 1-27 | 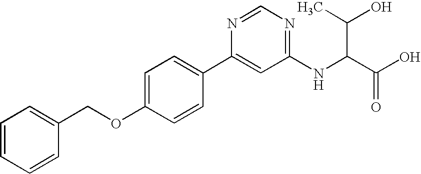 | 379.42 | 380 | 211-214 | B |
| 1-28 | 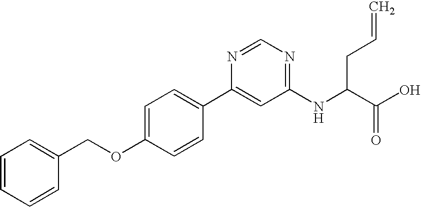 | 375.43 | 376 | 135-137 | A |
| 1-29 | 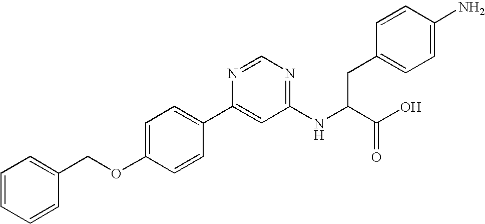 | 440.51 | 441 | 145 | A |
| 1-30 | 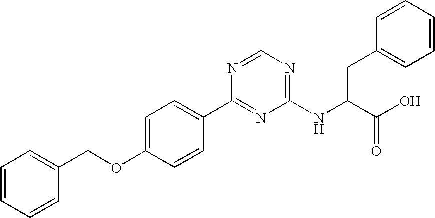 | 426.48 | 427 | 186-188 | A |
| 1-31 | 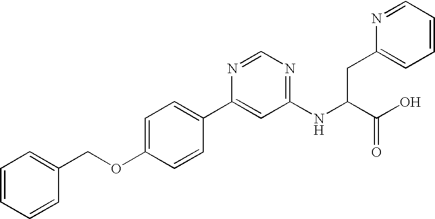 | 426.48 | 427 | 218-223 | A |
| 1-32 | 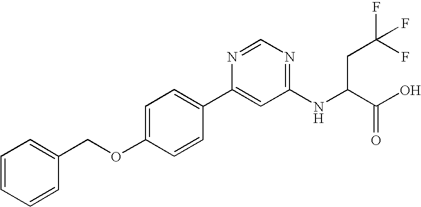 | 417.39 | 418 | 156-158 | A |
| 1-33 | 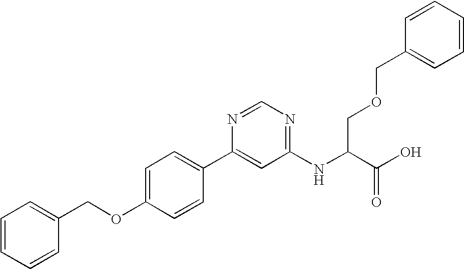 | 455.52 | 456 | 132-135 | A |
| 1-34 | 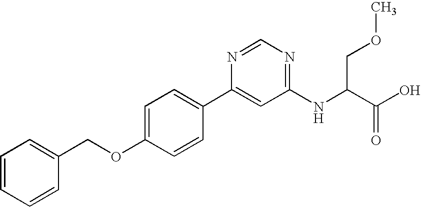 | 379.42 | 380 | 115-120 | A |
| 1-35 | 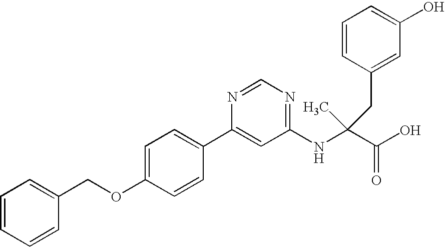 | 455.52 | 456 | 265-267 | C |
| 1-36 | 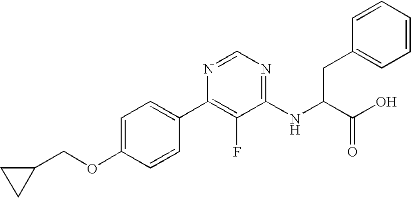 | 407.45 | 408 | 170-173 | A |
| 1-37 | 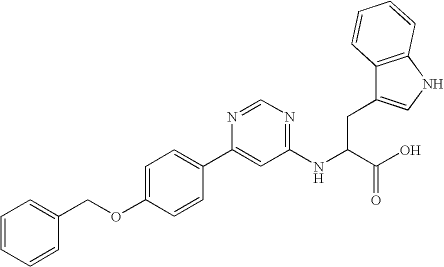 | 464.53 | 465 | 205-507 | A |
| 1-38 | 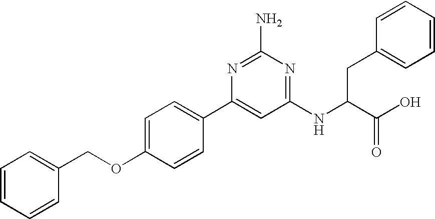 | 440.51 | 441 | 245Z | A |
| 1-39 | 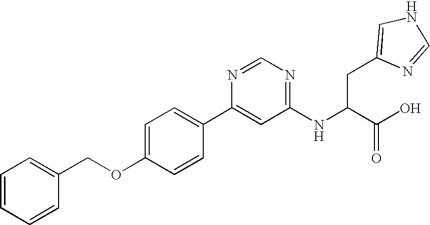 | 415.46 | 416 | 183-188 | B |
| 1-40 | 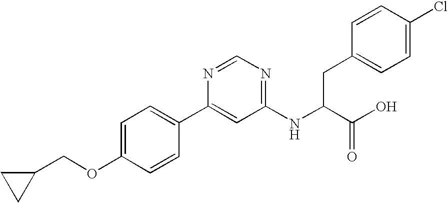 | 423.90 | 424 | 166-169 | A |
| 1-41 | 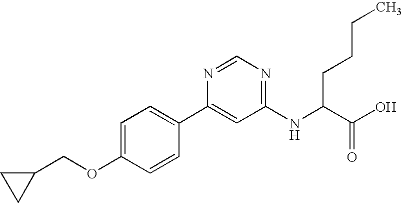 | 355.44 | 356 | 100-104 | B |
| 1-42 | 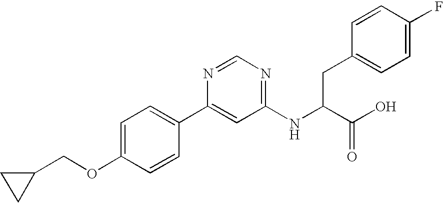 | 407.45 | 408 | 126-128 | A |
| 1-43 | 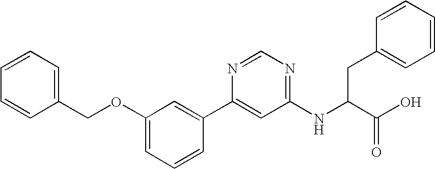 | 425.49 | 426 | 150-152 | C |
| 1-44 | 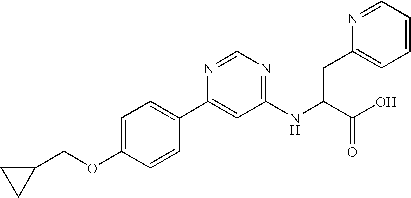 | 390.45 | 391 | 105-107 | B |
| 1-45 | 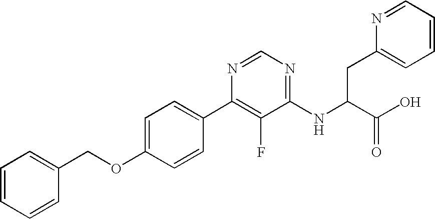 | 444.47 | 445 | 177-178 | A |
| 1-46 | 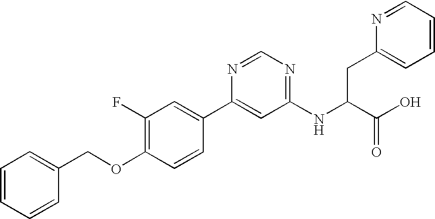 | 444.47 | 445 | 107 | A |
| 1-47 | 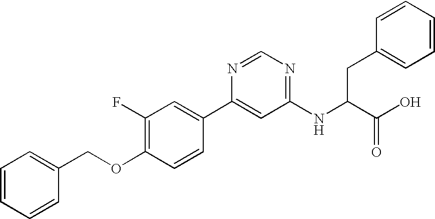 | 443.48 | 444 | 117-120 | A |
| 1-48 | 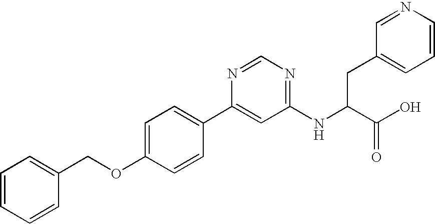 | 426.48 | 427 | 188-192 | A |
| 1-49 | 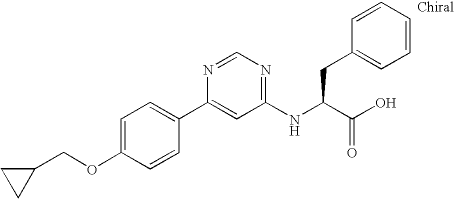 | 389.46 | 390 | 180-185 | C |
| 1-50 | 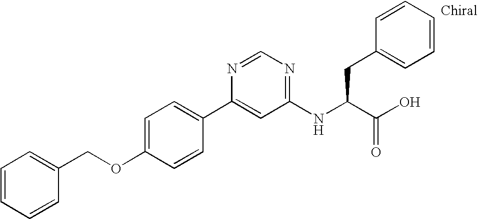 | 425.49 | 426 | 150-155 | B |
| 1-51 | 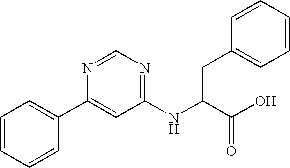 | 319.37 | 320 | 117Z | B |
| 1-52 | 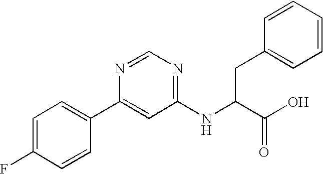 | 337.36 | 338 | 221Z | B |
| 1-53 | 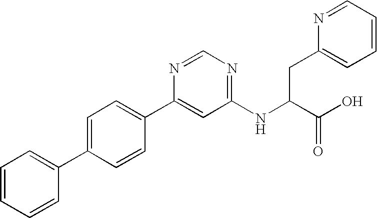 | 396.45 | 397 | amorphous | A |
| 1-54 | 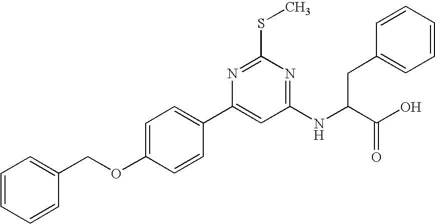 | 471.58 | 472 | 106Z | B |
| 1-55 | 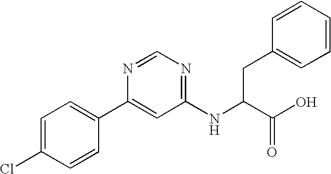 | 353.81 | 354 | 113Z | B |
| 1-56 | 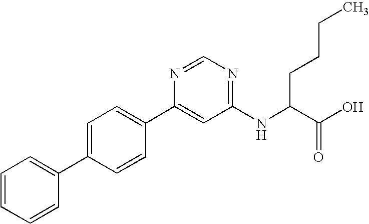 | 361.45 | 362 | 110-120 | B |
| 1-57 | 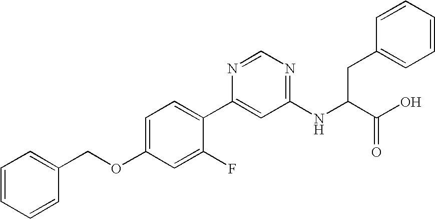 | 443.48 | 444 | 133-136 | B |
| 1-58 | 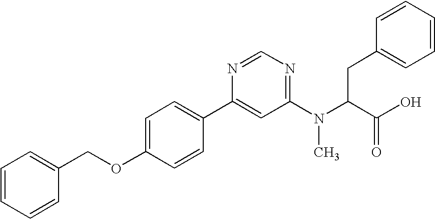 | 439.52 | 440 | 112-115 | A |

### Example 2-1

### Methyl N-[6-(4-hydroxyphenyl) pyrimidin-4-yl]phenylalaninate

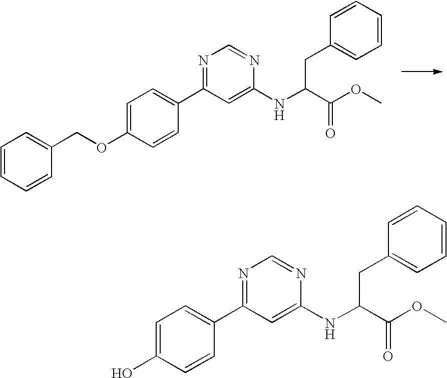

A mixture of methyl N-{6-[4-(benzyloxy)phenyl] pyrimidin-4-yl}phenylalaninate (0.253 g, 0.576 mmol), 10% palladium on activated carbon (0.050 g) and methanol (10 mL) under a hydrogen atmosphere was stirred at room temperature for 2 days. The resulting mixture was filtered through a Celite pad, and the filtrate was concentrated under reduced pressure. The residue was purified by column chromatography on silica-gel (hexane:ethyl acetate, 1:1) to give methyl N-[6-(4-hydroxyphenyl) pyrimidin-4-yl]phenylalaninate (0.150 g, 75%) as a colorless oil.

### Methyl N-{6-[4-(cyclopropylmethoxy)phenyl] pyrimidin-4-yl}phenylalaninate

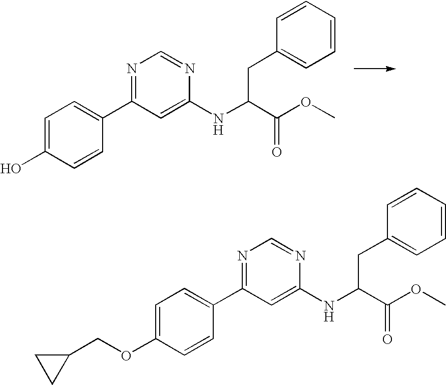

To a mixture of methyl N-[6-(4-hydroxyphenyl) pyrimidin-4-yl]phenylalaninate (0.020 g, 0.057 mmol), potassium carbonate (0.016 g, 0.11 mmol), acetone (1.0 mL) and DMF (1.0 mL) was added (bromomethyl)cyclopropane (0.008 mL, 0.09 mmol), and the mixture was stirred at reflux overnight. After cooled to room temperature, the mixture was filtered and the filtrate was concentrated under reduced pressure. The residue was purified by preparative TLC (hexane; ethyl acetate, 1:1) to give methyl N-{6-[4-(cyclopropylmethoxy)phenyl]pyrimidin-4-yl}phenylalaninate (0.024 g, 100%)as an yellow oil.

### N-{6-[4-(Cyclopropylmethoxy)phenyl]pyrimidin-4-yl}phenylalanine

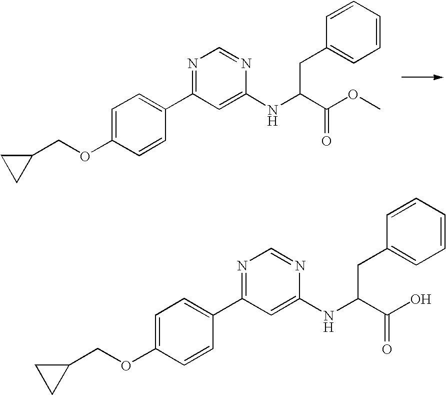

To a solution of Methyl N-{6-[4-(cyclopropylmethoxy)phenyl] pyrimidin-4-yl}-phenylalaninate (0.024 g, 0.059 mmol) in methanol (2.0 mL) was added 1M NaOH aqueous solution (0.5 mL), and stirring was continued at room temperature overnight. After removal of methanol under reduced pressure, the residue was diluted with water. The solution was washed with diethyl ether and acidified by 1M aqueous hydrochloric acid. The resulting precipitate was collected by filteration, washed with ethyl acetate, and dried under reduced pressure to give N-{6-[4-(cyclopropylmethoxy)phenyl] pyrimidin-4-yl}phenylalanine (0.018 g, 77%) as a colorless solid.

Melting point: 216-218° C.

Molecular weight: 389.45

Mass spectrometry: 390 (M+H)⁺

In vitro activity grade: A

¹H-NMR (500 MHz, MeOH-d4): δ 0.38(2H, m), 0.64 (2H, m), 1.28 (1H, m), 3.12 (1H, dd, J=9.1, 13.9 Hz), 3.42 (1H, dd, J=4.7, 13.6 Hz), 3.92 (2H, d, J=6.9 Hz), 5.21 (1H, m), 6.96 (1H, s), 7.12 (2H, d, J=8.8 Hz), 7.21 (1H, m), 7.26 (4H, m), 7.73 (2H, d, J=8.5 Hz), 8.58 (1H, s).

### Examples 2-2 to 2-46

In the similar manners as described in Example 2-1 above, compounds in Examples 2-2 to 2-46 as shown in Table 2 were synthesized.

TABLE EXAMPLE 2

| | | | MASS | | |
|---|---|---|---|---|---|
| Ex. No | Structure | M.W. | (M + 1) | MP | In vitro |
| 2-2 | 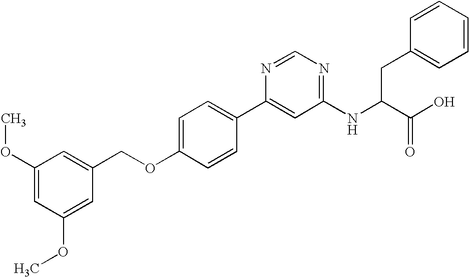 | 485.54 | 486 | 90-93 | A |
| 2-3 | 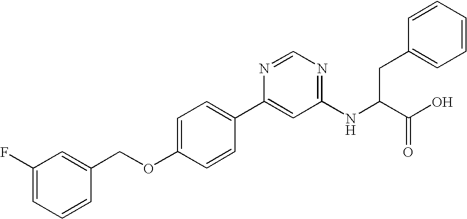 | 443.48 | 444 | 175-177 | A |
| 2-4 | 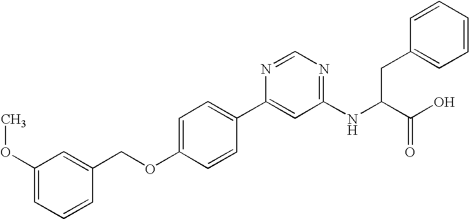 | 455.52 | 456 | 90-93 | A |
| 2-5 | 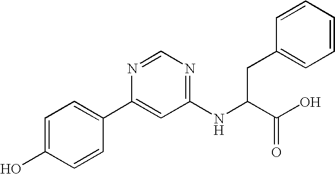 | 335.37 | 336 | 223-227 | C |
| 2-6 | 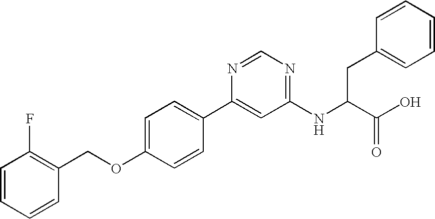 | 443.48 | 444 | 220-223Z | A |
| 2-7 | 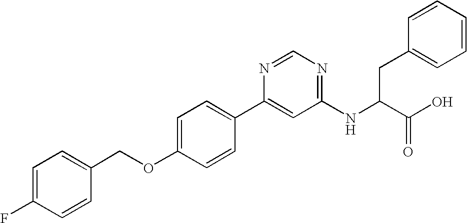 | 443.48 | 444 | 228-230Z | A |
| 2-8 | 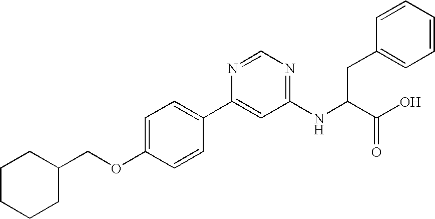 | 431.54 | 432 | 118-120 | A |
| 2-9 | 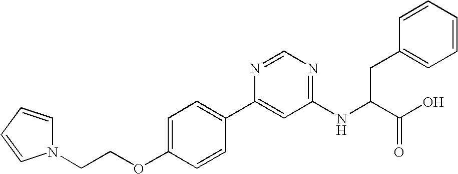 | 428.50 | 429 | 209-211 | A |
| 2-10 | 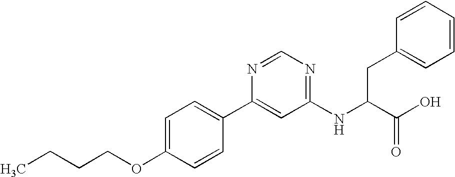 | 391.47 | 392 | 205-208 | A |
| 2-11 | 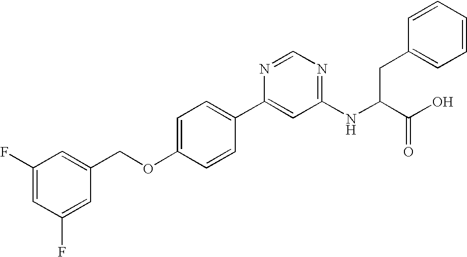 | 461.47 | 462 | 207-210Z | A |
| 2-12 | 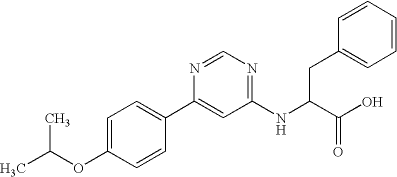 | 377.45 | 378 | 201-204Z | A |
| 2-13 | 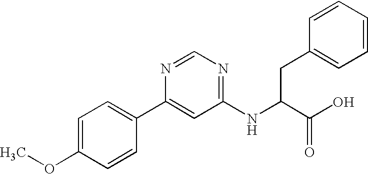 | 349.39 | 350 | 203-206Z | B |
| 2-14 | 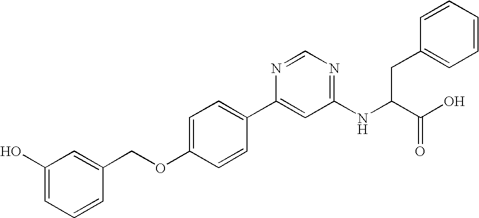 | 441.49 | 442 | 190-193Z | A |
| 2-15 | 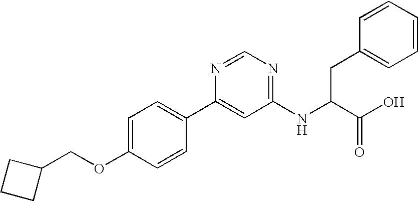 | 403.49 | 404 | 210-213Z | A |
| 2-16 | 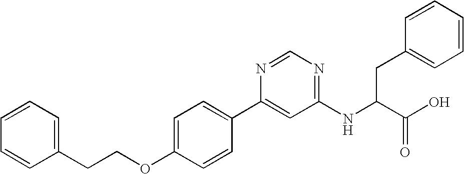 | 439.52 | 440 | 214-216Z | A |
| 2-17 | 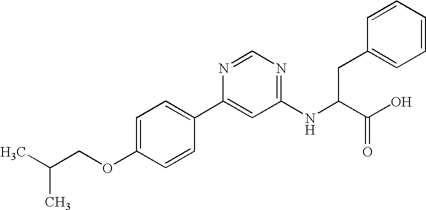 | 391.47 | 392 | 210-213Z | A |
| 2-18 | 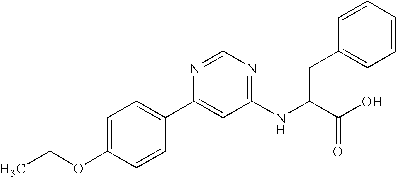 | 363.42 | 364 | 190Z | A |
| 2-19 | 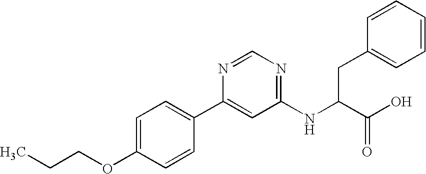 | 377.45 | 378 | 205Z | A |
| 2-20 | 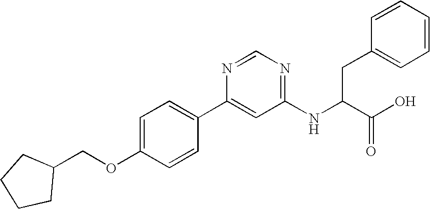 | 417.51 | 418 | 125-128 | A |
| 2-21 | 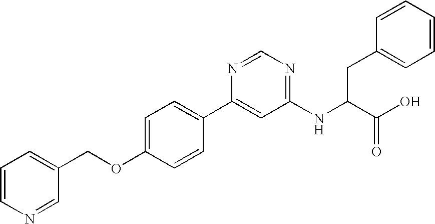 | 426.48 | 427 | 135-136Z | A |
| 2-22 | 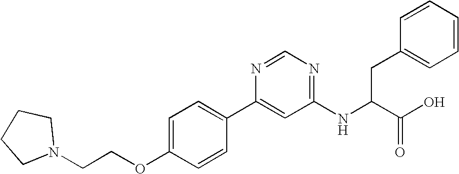 | 432.53 | 433 | 117-120 | C |
| 2-23 | 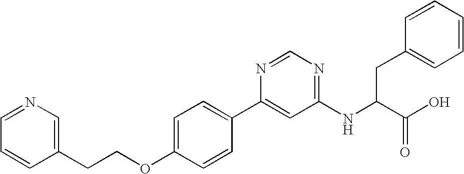 | 440.51 | 441 | 85-88 | A |
| 2-24 | 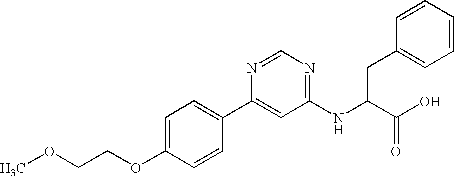 | 393.45 | 394 | 190-193Z | A |
| 2-25 | 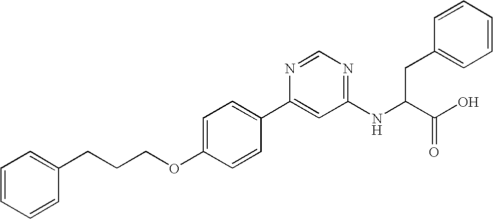 | 453.55 | 454 | 101-104 | A |
| 2-26 | 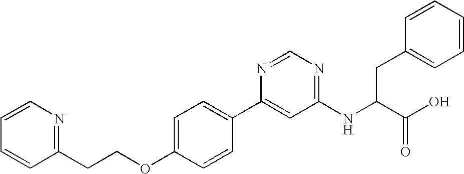 | 440.51 | 441 | 120-122Z | A |
| 2-27 | 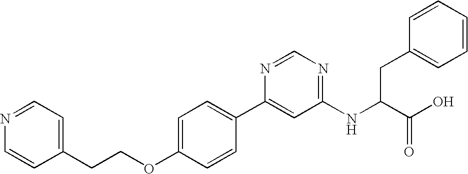 | 440.51 | 441 | 113-115 | A |
| 2-28 | 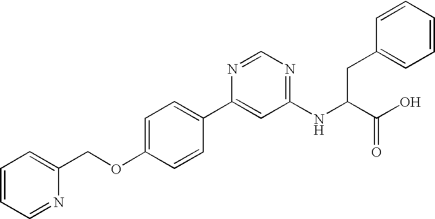 | 426.48 | 427 | 120-123 | A |
| 2-29 | 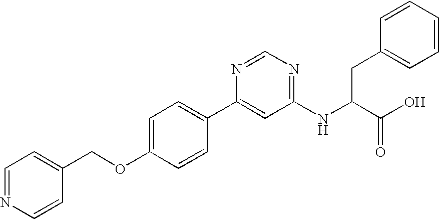 | 426.48 | 427 | 110-113 | A |
| 2-30 | 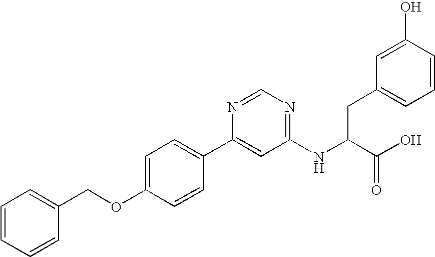 | 441.49 | 442 | 169 | A |
| 2-31 | 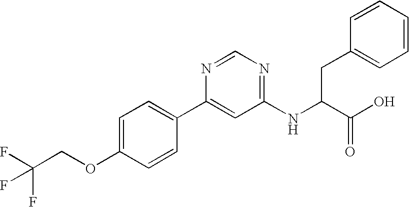 | 417.39 | 418 | 109-112 | A |
| 2-32 | 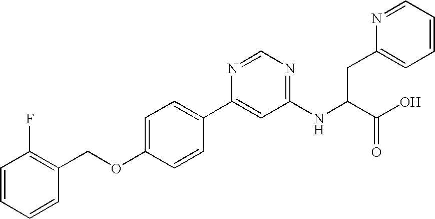 | 444.47 | 445 | 84-87 | A |
| 2-33 | 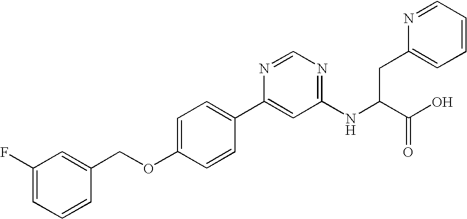 | 444.47 | 445 | 92-95 | A |
| 2-34 | 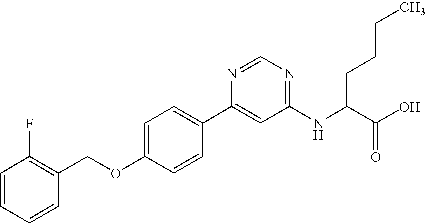 | 409.46 | 410 | 85-90 | A |
| 2-35 | 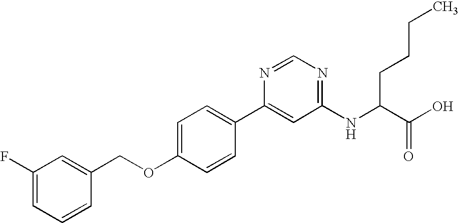 | 409.46 | 410 | 181-186 | A |
| 2-36 | 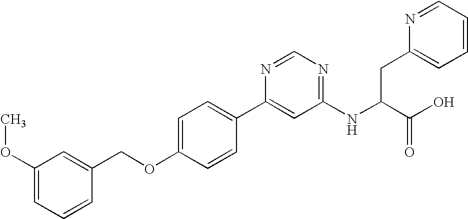 | 456.51 | 457 | 109Z | A |
| 2-37 | 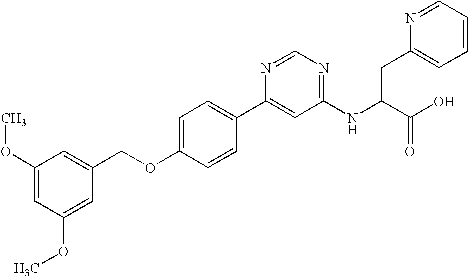 | 486.53 | 487 | 99Z | A |
| 2-38 | 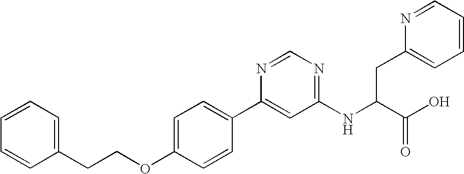 | 440.51 | 441 | 52-53 | A |
| 2-39 | 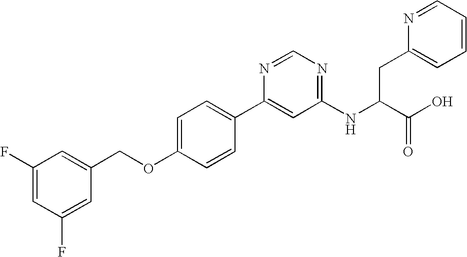 | 462.46 | 463 | 90-92 | A |
| 2-40 | 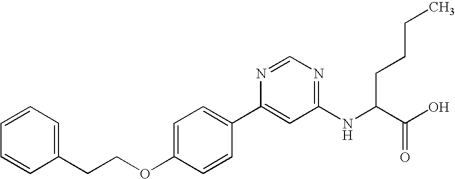 | 405.50 | 406 | 86-88 | A |
| 2-41 | 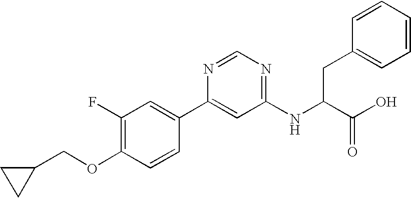 | 407.45 | 408 | oil | A |
| 2-42 | 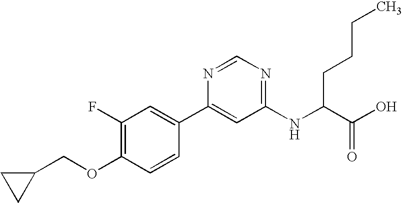 | 373.43 | 374 | oil | B |
| 2-43 | 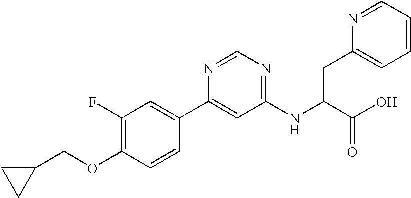 | 408.44 | 409 | 98Z | B |
| 2-44 | 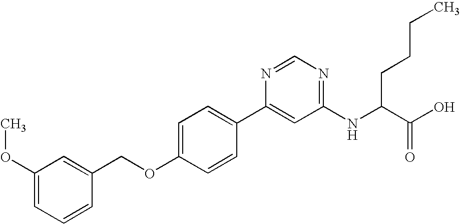 | 421.50 | 422 | 115-118 | A |
| 2-45 | 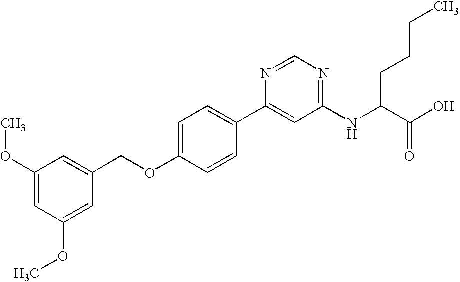 | 451.53 | 452 | 88-93 | A |
| 2-46 | 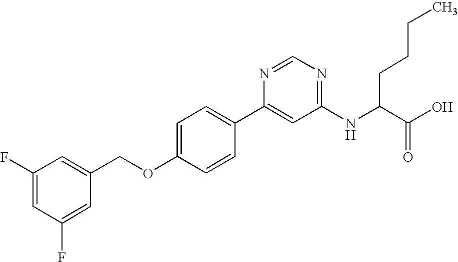 | 427.45 | 428 | 141-147 | A |

### Example 3-1

### Methyl N-(2-chloro-4-pyrimidinyl)phenylalaninate

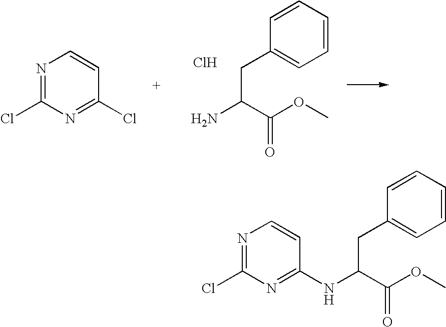

To a mixture of 2,4-dichloropyrimidine (0.800 g, 4.85 mmol), DL-phenylalanine methyl ester hydrochloride (1.098 g, 5.090 mmol) and ethanol (15 mL) was added N,N-diisopropylethylamine (1.773 mL, 10.18 mmol), and the mixture was stirred at reflux for 6 hours. After cooled to room temperature, the precipitate was removed by filtration and washed with ethanol. The combined filtrates were concentrated under reduced pressure, and the residue was partitioned between ethyl acetate and water. The separated organic phase was washed with brine, dried over sodium sulfate, filtered and concentrated under reduced pressure. The crude product was purified by column chromatography on silica-gel (hexane:ethyl acetate, 2:1) to give methyl N-(2-chloro-4-pyrimidinyl)phenylalaninate (1.020 g, 72%) as a colorless oil.

### Methyl N-{2-[4-(benzyloxy)phenyl]-4-pyrimidinyl}phenylalaninate

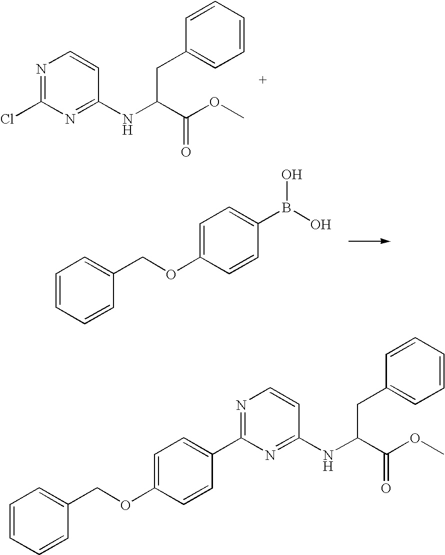

To a mixture of methyl N-(2-chloro-4-pyrimidinyl)phenylalaninate (0.368 g, 1.261 mmol), 4-(benzyloxy)phenylboronic acid (0.316 g, 1.388 mmol) and DMF (5 mL)under an argon atmosphere was added a solution of sodium carbonate (0.414 g, 3.910 mmol) in water (2 mL) followed by tetrakis(triphenylphosphine)palladium (0.068 g, 0.059 mmol). The mixture was stirred at 95° C. overnight. After cooled to room temperature, the mixture was treated with 1N aqueous sodium hydroxide solution (2 mL) and stirred at room temperature for 2 hours. The mixture was diluted with water, and washed with ethyl acetate. The separated aqueous phase was neutralized by 1N aqueous hydrochloric acid solution. The resultant precipitate was collected by filtration, washed with water and dried under reduced pressure. The residue was dissolved in a mixture of methylene chloride (10 mL) and methanol (10 mL), and treated with a solution of diazomethane in ether, which was prepared from 1-methyl-3-nitro-1-nitrosoguanidine (0.5 g, 3.4 mmol), potassium hydroxide (6 g), water (9 g) and ether (25 mL). After being stirred for 1 hour, the mixture was concentrated under reduced pressure. The crude product was purified by column chromatography on silica-gel (hexane:ethyl acetate, 2:1) to give methyl N-{2-[4-(benzyloxy)phenyl]-4-pyrimidinyl}phenylalaninate (0.223 g, 40%) as a colorless oil.

### N-{2-[4-(Benzyloxy)phenyl]-4-pyrimidinyl}phenylalanine

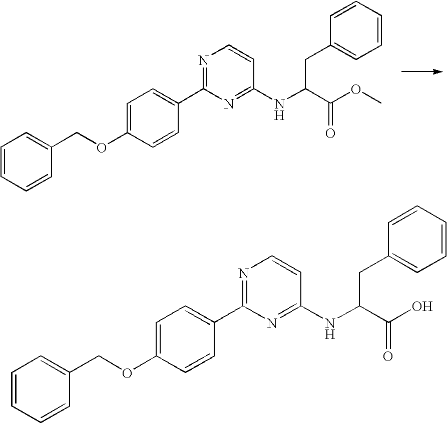

To a solution of methyl N-{2-[4-(benzyloxy)phenyl]4-pyrimidinyl}phenylalaninate (0.220 g, 0.501 mmol) in methanol (2.0 mL), water (2.0 mL) and tetrahydrofuran (4.0 mL), and the mixture was stirred at room temperature for 2 hours. The mixture was diluted with water (5 mL). The mixture was neutralized with 1N hydrochloric acid solution (0.715 mL). The resultant crystal was collected by filtration, washed with water and ether, and dried under reduced pressure to give N-{2-[4-4-(benzyloxy)phenyl]4-pyrimidinyl}phenylalanine (0.178 g, 73%) as a white solid.

Melting point: 120-125° C.

Molecular weight: 425.49

Mass spectrometry: 426 (M+H)⁺

In vitro activity grade: B

¹H-NMR (500 MHz, DMSO-d6): δ 3.04 (1H, dd, J=9.3, 13.9 Hz), 3.19 (1H, dd, J=5.0, 13.9 Hz), 4.75 (1H, br), 5.17 (2H, s), 6.45 (1H, d, J=5.5 Hz), 7.07 (2H, d, J=9.0 Hz), 7.19 (1H, dd, J=6.9, 7.1 Hz), 7.25-7.36 (5H, m), 7.40 (2H, dd, J=7.1, 7.7 Hz), 7.47 (2H, d, J=7.1 Hz), 7.75 (1H, br), 8.11 (1H, d, J=5.8 Hz), 8.23 (2H, d, J=8.8 Hz), 12.66 (1H, br s).

### Examples 3-2 to 3-4

In the similar manners as described in Example 3-1 above, compounds in Examples 3-2 to 3-4 as shown in Table 3 were synthesized.

TABLE EXAMPLE 3

| | | | MASS | | |
|---|---|---|---|---|---|
| Ex. No. | Structure | M.W. | (M + 1) | MP | In vitro |
| 3-2 | 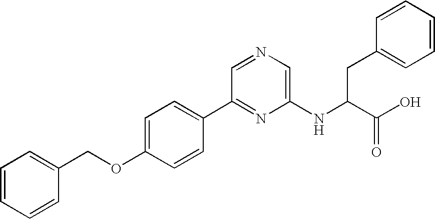 | 425.49 | 426 | 89-92 | C |
| 3-3 | 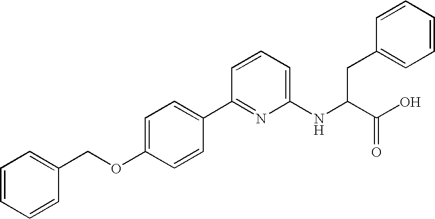 | 424.50 | 425 | 81-84 | C |
| 3-4 | 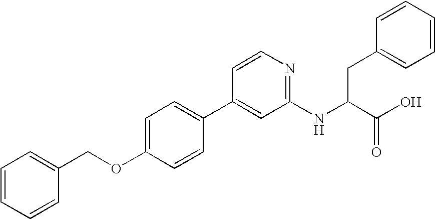 | 424.50 | 425 | 194-196 | A |

### Example 4-1

### Ethyl N-{6-[4-(benzyloxy)phenyl]pyrimidin-4-yl}-3-[2-(dimethylamino)ethoxy] phenylalaninate

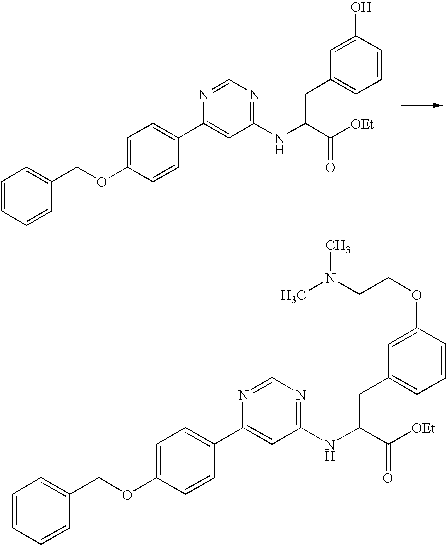

A mixture of ethyl N-{6-[4-(benzyloxy)phenyl]pyrimidin-4-yl}-3-hydroxyphenyl-alaninate (44.0 mg, 0.09 mmol), (2-chloroethyl)dimethylamine hydrochloride (16.2 mg, 0.11 mmol) and potassium carbonate (32.4 mg, 0.23 mmol) in DMF (0.5 mL) was stirred at 60° C. overnight and at 90° C. for 4 hours. After cooled to room temperature, the mixture was concentrated under reduced pressure. The residue was diluted with ethyl acetate and the precipitate was filtered off. The filtrate was purified by preparative TLC (CH₂Cl₂/MeOH/conc.NH₃, 100/10/1) to give ethyl N-{6-[4-(benzyloxy) phenyl]pyrimidin-4-yl}-3-[2-(dimethylamino)ethoxy]phenylalaninate (30.0 mg, 59%) as a gum.

### N-{6-[4-(Benzyloxy)phenyl]pyrimidin-4-yl}-3-[2-(dimethylamino)ethoxy]phenylalanine

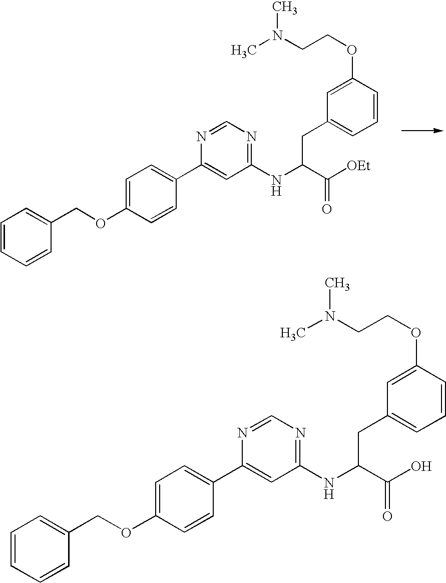

To a solution of N-{6-[4-(benzyloxy) phenyl]pyrimidin-4-yl}-3-[2-(dimethylamino)ethoxy]phenylalaninate (30 mg, 0.060 mmol) in THF (0.1 mL) was added 1N LiOH aqueous solution (0.08 mL, 0.08 mmol) and the mixture was stirred at room temperature overnight. The mixture was neutralized with 1N HCl (0.08 mL) and concentrated under reduced pressure. The residue was purified by reversed phase preparative TLC (Merck RP-18, CH3CN/water, 2/1) followed by crystallization from ethyl ether to give N-{6-[4-(benzyloxy)phenyl]pyrimidin-4-yl}-3-[2-(dimethylamino)ethoxy]phenylalanine (10.1 mg, 36%) as a colorless powder.

Melting point: 143.1° C.

Molecular weight: 512.61

Mass spectrometry: 513 (M+H)⁺

In vitro activity grade: A

¹H-NMR (500 MHz, DMSO-d6): δ 2.22 (6H, s), 2.63 (2H, bs), 2.97 (1H, dd, J 13.2, 9.5 Hz), 3.17 (1H, d, J=10.1 Hz), 4.00 (2H, t, J=5.7 Hz), 4.75 (1H, bs), 5.17 (2H, s), 6.75 (1H, d, J=7.9 Hz), 6.84 (1H, d, J=7.5 Hz), 6.85 (1H, s), 6.99 (1H, s), 7.12 (2H, d, J=7.2 Hz), 7.16 (1H, t, J=7.9 Hz), 7.34 (1H, t, J=7.2 Hz), 7.40 (2H, t, J=7.0 Hz), 7.47 (2H, d, J=7.5 Hz), 7.56 (1H, bs), 7.93 (2H, d, J=7.6 Hz), 8.43 (1H, bs).

### Example 4-2

In the similar manners as described in Example 4-1 above, compound in Example 4-2 as shown in Table 4 was synthesized.

TABLE EXAMPLE 4

| | | | MASS | | |
|---|---|---|---|---|---|
| Ex. No | Structure | M.W. | (M + 1) | MP | In vitro |
| 4-2 | 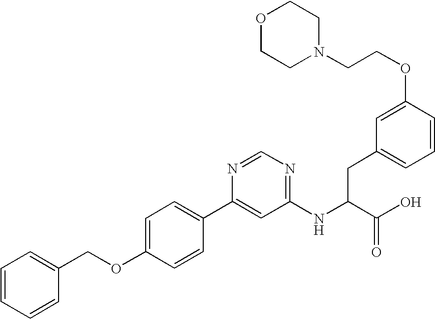 | 554.65 | 555 | 111 | A |

### Example 5-1

### Ethyl N-{6-[4-(benzyloxy)phenyl]pyrimidin-4-yl}-3-{2-[(tert-butoxycarbonyl)amino]ethoxy}phenylalaninate

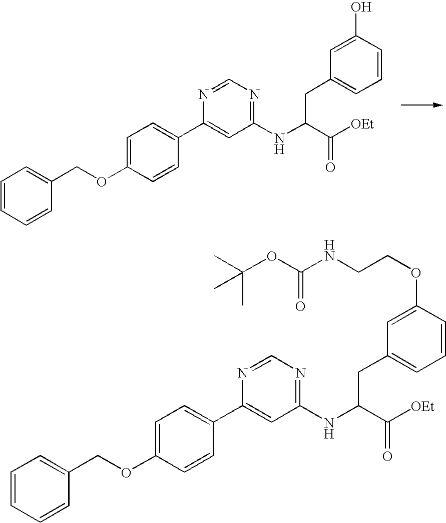

A mixture of ethyl N-{6-[4-(benzyloxy)phenyl]pyrimidin-4-yl}-3-hydroxyphenylalaninate (150.0 mg, 0.32 mmol), tert-butyl (2-bromoethyl)carbamate (107.4 mg, 0.48 mmol) and potassium carbonate (66.2 mg, 0.48 mmol) and DMF (1.0 mL) was stirred at room temperature for 2 days. The mixture was partitioned between ethyl acetate and water. The separated organic layer was washed with brine, dried over sodium sulfate, filtered and concentrated under reduced pressure. The residue was purified by preparative TLC (CHCl₃/MeOH, 19/1; then CHCl₃/AcOEt 2/1)) to give ethyl N-{6-[4-(benzyloxy)phenyl]pyrimidin-4-yl}-3-{2-[(tert-butoxycarbonyl) amino]-ethoxy}phenyl alaninate (47.0 mg, 24%) as a gum.

### 3-(2-Aminoethoxy)-N-{6-[4-(benzyloxy)phenyl]pyrimidin-4-yl}phenylalanine

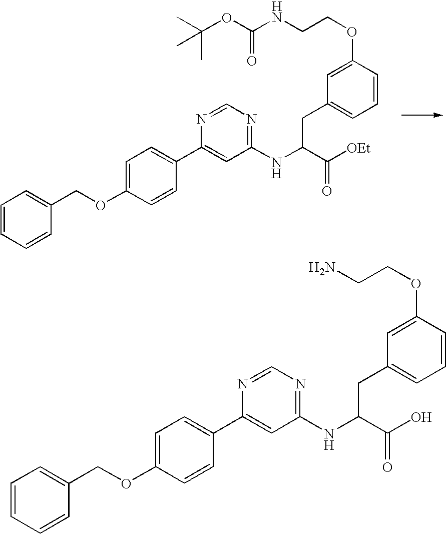

To a solution of ethyl N-{6-[4-(benzyloxy)phenyl]pyrimidin-4-yl}-3-{2-[(tertbutoxycarbonyl)amino]ethoxy}phenyl alaninate (47.0 mg, 0.080 mmol) in ethanol (11.0 mL) was added 1N LiOH aqueous solution (0.12 mL, 0.12 mmol) and the mixture was stirred at room temperature for 3 hours. The mixture was neutralized with 1N HCl (0.12 mL) and partitioned between ethyl acetate and water, washed with brine, dried over sodium sulfate and concentrated under reduced pressure. The residue was dissolved in methanol (1.0 mL). A 4N solution of HCl in dioxane(1.0 mL, 4.0 mmol) was added to the solution, which was kept at room temperature overnight. The volatiles were evaporated off, and the residual solid was tirturated with ethyl ether to give a crude powder, which was recrystallized from a mixture of THF and water to give 3-(2-aminoethoxy)-N-{6-[4-(benzyloxy)phenyl]pyrimidin-4-yl}phenylalanine (13.7 mg, 37%) as a colorless powder.

Melting point: 153.8° C.

Molecular weight: 484.55

Mass spectrometry: 485 (M+H)⁺

In vitro activity grade: A

¹H-NMR (500 MHz, CD₃OD): δ 3.11 (1H, dd, J=14.0, 8.8 Hz), 3.33 (2H, t, J=5.1 Hz), 3.37 (1H, dd, J=14.1 Hz, 5.2 Hz), 4.19 (1H, t, J=4.9 Hz), 5.13 (1H, bs), 5.20 (2H, s), 6.87 (1H, d, J=8.2 Hz), 6.93-6.96 (3H, m), 7.18 (2H, d, J=8.8 Hz), 7.23 (1H, t, J=7.9 Hz), 7.32 (1H, t, J=7.3 Hz), 7.38 (2H, t, J=7.4 Hz), 7.45 (2H, d, J=7.9 Hz), 7.78 (2H, d, J=9.1 Hz), 8.54 (1H, s).

### Example 6-1

### Ethyl N-{6-[4-(benzyloxy)phenyl]pyrimidin-4-yl}-4-(4,5-dihydro-1H-imidazol-2-ylamino)phenylalaninate

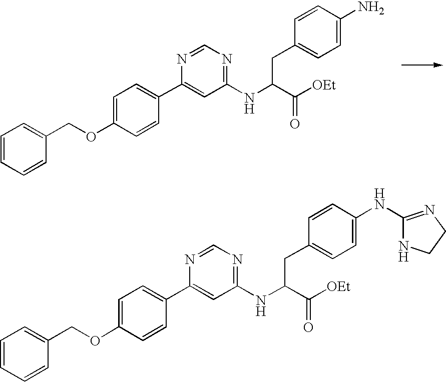

A mixture of ethyl 4-amino-N-{6-[4-(benzyloxy)phenyl]pyrimidin-4-yl}phenyl alaninate (65.0 mg, 0.14 mmol), methyl 2-(methylthio)-4,5-dihydro-1H-imidazole-1-carboxylate (29.0 mg, 0.17 mmol) in acetic acid (0.20 mL) and ethanol (2.0 mL) was stirred at 65° C. for 2 days. After cooled to room temperature, the mixture was concentrated under reduced pressure. The residue was purified by preparative TLC (CH₂Cl₂/MeOH/conc.NH₃, 100/10/1) to give ethyl N-{6-[4-(benzyloxy)phenyl]pyrimidin-4-yl}-4-(4,5-dihydro-1H-imidazol-2-ylamino)phenylalaninate (49.0 mg, 66%) as a gum.

### N-{6-[4-(Benzyloxy)phenyl]pyrimidin-4-yl}-4-(4,5-dihydro-1H-imidazol-2-yl-amino)phenylalanine

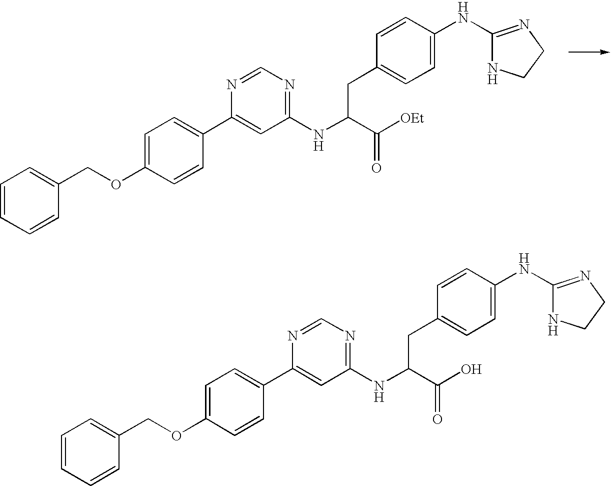

To an iced solution of ethyl N-{6-[4-(benzyloxy)phenyl]pyrimidin-4-yl}-4-(4,5-dihydro-1H-imidazol-2-ylamino)phenylalaninate (49.0 mg, 0.09 mmol) in tetrahydrofuran (1.0 mL) was added 1N LiOH (0.14 mL, 0.14 mmol) and the mixture was stirred at rt for 5 hours. After neutralized with 1N HCl (0.147 mL), the mixture was concentrated under reduced pressure to the driness. The residue was purified by HP-20 column chromatography (water ->MeOH) followed by trituration with ethyl ether to give N-{6-[4-(benzyloxy)phenyl]pyrimidin-4-yl}-(4,5-dihydro-1H-imidazol-2-ylamino)phenylalanine (23.0 mg, 50%) as an ivory powder.

Melting point: 169.2° C. (dec.)

Molecular weight: 508.58

Mass spectrometry: 509 (M+H)⁺

In vitro activity grade: A

¹H-NMR (500 MHz, CD₃OD): δ 3.12 (1H, dd, J=13.8, 7.3 Hz), 3.33 (1H, m), 3.74 (4H, m), 5.16 (2H, s), 6.84 (1H, bs), 7.10 (2H, d, J=8.8 Hz), 7.15 (2H, d, J=8.2 Hz), 7.30-7.39 (5H, m), 7.45 (2H, d, J=7.2 Hz), 7.84 (2H, d, J=8.8 Hz), 8.40 (1H, s).

### Example 7-1

### Methyl N-[6-(4-{[(trifluoromethyl)sulfonyl]oxy}phenyl)pyrimidin-4-yl]phenylalaninate

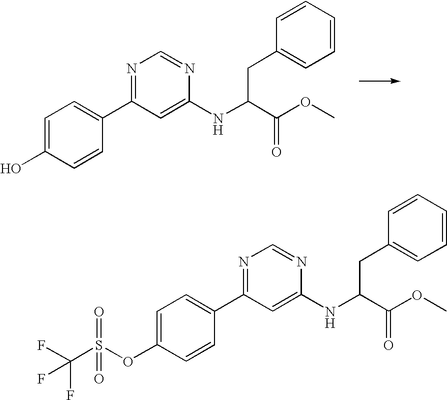

To a solution of methyl N-[6-(4-hydroxyphenyl)-4-pyrimidinyl]phenylalaninate (0.03 g, 0.09 mmol) and triethylamine (0.03 mL, 0.19 mmol) in dichloromethane (2 mL) was added trifluoromethanesulfonic anhydride (0.04 mL, 0.26 mmol) at 0° C., and the mixture was stirred at room temperature for 4 hours. The mixture was diluted with water and extracted with chloroform. The separated organic phase was washed with brine, dried over sodium sulfate, filtered and concentrated under reduced pressure. The residue was purified by column chromatography on silica-gel (hexane:ethyl acetate, 3:1) to give methyl N-[6-(4-{[(trifluoromethyl)sulfonyl]oxy}-phenyl) pyrimidin-4-yl]phenylalaninate (39 mg, 94%) as slightly yellow oil.

### Methyl N-(6-{4-[(E)-2-phenylvinyl]phenyl}pyrimidin-4-yl)phenylalaninate

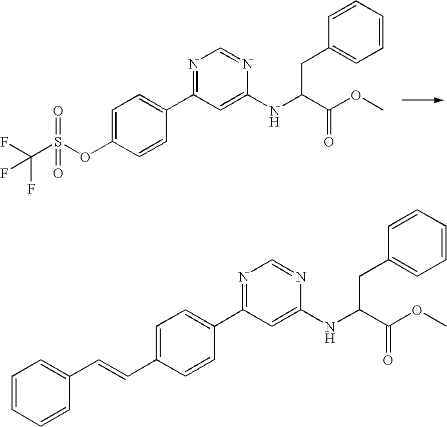

To a solution of methyl N-[6-(4-{[(trifluoromethyl)sulfonyl]oxy}phenyl)pyrimidin-4-yl]phenylalaninate (0.046 g, 0.10 mmol) and trimethylamine (0.04 mL, 0.29 mmol) in N,N-dimethylformamide (2 mL) was added tetrakis(triphenylphosphine)palladium (0.09 g, 0.01 mmol) and styrene (0.020 mL, 0.19 mmol), and the mixture was stirred at 80° C. overnight. After cooled to room temperature, the mixture was partitioned between ethyl acetate and water. The separated organic phase was washed with brine, dried over sodium sulfate, filtered and concentrated under reduced pressure. The crude product was purified by preparative TLC (silica-gel, hexane:ethyl acetate, 3:2) to give methyl N-(6-{4-[(E)-2-phenylvinyl]phenyl}pyrimidin-4-yl) phenylalaninate (0.0126 g, 30%) as colorless solid.

### N-(6-{4-[(E)-2-Phenylvinyl]phenyl}pyrimidin-4-yl)phenylalanine

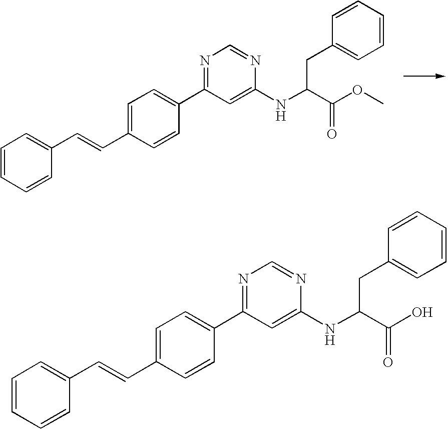

To a solution of methyl N-(6-{4-[(E)-2-phenylvinyl]phenyl}pyrimidin-4-yl) phenylalaninate (0.016 g, 0.04 mmol) in methanol (2 m) was added 1M NaOH aqueous solution (0.5 mL) at room temperature, and the mixture was stirred for 3 hours. After the removal of methanol under reduced pressure, the residue was diluted with water. The solution was washed with diethyl ether and acidified by 1N aqueous hydrochloric acid. The resulting precipitates were collected by filtration and dried under reduced pressure to give N-(6-{4-[(E)-2-phenylvinyl]phenyl}pyrimidin-4-yl)phenylalanine (0.014 g, 92%) as colorless solid.

Melting point: 214-216° C. (dec.)

Molecular weight: 421.503

Mass spectrometry: 422 (M+H)⁺

In vitro activity grade: A

¹H-NMR (500 MHz, MeOH-d4): δ 3.15 (1H, dd, J=8.8, 13.9 Hz), 3.44 (1H, dd, J=4.4, 13.9 Hz), 5.24 (1H, m), 7.06 (1H, br s), 7.21 (1H, m),7.29 (6H, m), 7.37 (2H, m), 7.40 (1H, d, J=5.0 Hz), 7.61 (2H, d, J=7.3 Hz), 7.80 (4H, m), 863 (1H, br s).

### Example 8-1

### Methyl N-{6-[4-(2-phenylethyl)phenyl]pyrimidin-4-yl}phenylalaninate

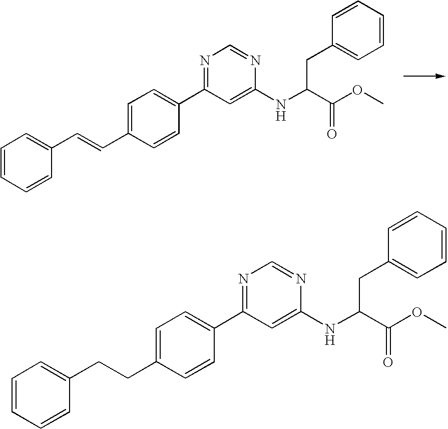

A mixture of methyl N-(6-{4-[(E)-2-phenylvinyl]phenyl}pyrimidin-4-yl) phenylalaninate (0.016 g, 0.04 mmol), 10% palladium on activated carbon (0.002 g) and methanol (1 mL) under a hydrogen atmosphere was stirred at room temperature for 4 hours. The resulting mixture was filtered through a Celite pad, and the filtrate was concentrated under reduced pressure. The residue was purified by preparative TLC (silica-gel, hexane:ethyl acetate, 1:1) to give methyl N-{6-[4-(2-phenylethyl)phenyl]pyrimidin-4-yl}phenylalaninate (0.014 g, 88%) as colorless oil.

### N-{6-[4-(2-Phenylethyl)phenyl]pyrimidinyl}phenylalanine

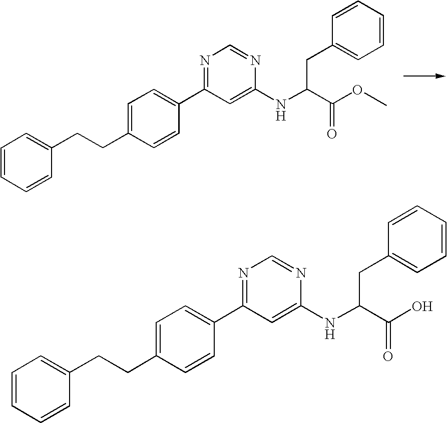

To a solution of methyl N-{6-[4-(2-phenylethyl)phenyl]pyrimidin-4-yl}phenylalaninate (0.013 g, 0.03 mmol) in methanol (2 mL) was added 1M NaOH aqueous solution (0.5 mL) at room temperature, and the mixture was stirred for 3 hours. After the removal of methanol under reduced pressure, water was added to the residue; The solution was washed with diethyl ether and acidified by 1N aqueous hydrochloric acid. The resulting precipitates were collected by filtration and dried under reduced pressure to give N-{6-[4-(2-phenylethyl)phenyl]pyrimidin-4-yl}phenylalanine (0.01 g, 85%) as colorless solid.

Melting point: 216-218° C.

Molecular weight: 423.519

Mass spectrometry: 424 (M+H)⁺

In vitro activity grade: A

¹H-NMR (500 MHz, MeOH-d4): δ 2.96 (2H, dd, J=7.6, 7.9 Hz), 3.04 (2H, dd, J=6.0, 7.9 Hz), 3.13 (1H, dd, J=9.5, 14.2 Hz), 3.42 (1H, m), 5.22 (1H, br s), 7.00 (1H, br s), 7.14-7.27 (10H, m), 7.40 (2H, d, J=8.2 Hz), 7.67 (2H, d, J=7.9 Hz), 8.59 (1H, br s).

### Example 9-1

### Methyl N-{6-[4-(phenylethynyl)phenyl]pyrimidin-4-yl}phenylalaninate

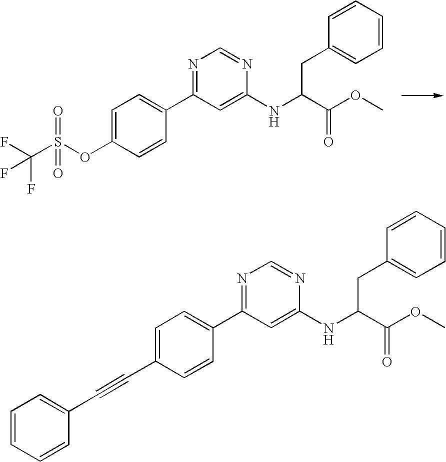

To a solution of methyl N-[6-(4-{[(trifluoromethyl)sulfonyl]oxy}phenyl)pyrimidin-4-yl]phenylalaninate (0.05 g, 0.10 mmol) and trimethylamine (0.04 mL, 0.31 mmol) in N,N-dimethylformamide (2 mL) was added tetrakis(triphenylphosphine)palladium (0.06 g, 0.01 mmol) and phenylacetylene (0.02 mL, 0.21 mmol), and the mixture was stirred at 80° C. for 7 hours. After cooled to room temperature, the mixture was partitioned between ethyl acetate and water. The separated organic phase was washed with brine, dried over sodium sulfate, filtered and concentrated under reduced pressure. The crude product was purified by column chromatography on silica-gel (hexane:ethyl acetate, 4:1) to give methyl N-{6-[4-(phenylethynyl)phenyl]-pyrimidin-4-yl}phenylalaninate (0.038 g, 85%) as slightly yellow oil.

### N-{6-[4-(Phenylethynyl)phenyl]pyrimidin-4-yl}phenylalanine

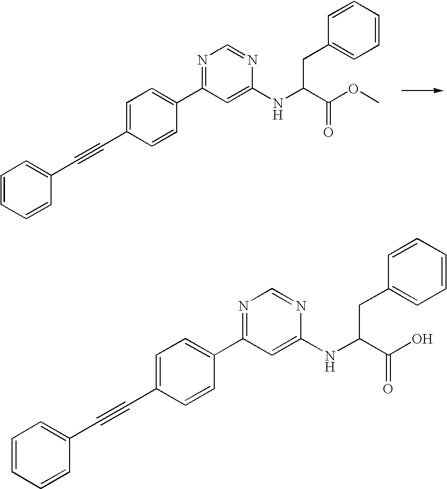

To a solution of methyl N-{6-[4-(phenylethynyl)phenyl]pyrimidin-4-yl}phenylalaninate (0.011 g, 0.030 mmol) in methanol (2 mL) was added 1M NaOH aqueous solution (0.5 mL) at room temperature, and the mixture was stirred for 3 hours. After the removal of methanol under reduced pressure, water was added to the residue. The solution was washed with diethyl ether and acidified by 1N aqueous hydrochloric acid. The resulting precipitates were collected by filtration and dried under reduced pressure to give N-{6-[4-(phenylethynyl)phenyl]pyrimidin-4-yl}phenylalanine (0.007 g, 67%) as a colorless solid.

Melting point: 215-218° C. (dec.)

Molecular weight: 419.487

Mass spectrometry: 420 (M+H)⁺

In vitro activity grade: A

¹H-NMR (500 MHz, MeOH-d4): δ 3.13 (1H, dd, J=8.8, 13.9 Hz), 3.41 (1H, dd, J=4.7, 13.9 Hz), 5.18 (1H, m), 7.03 (1H, br s), 7.20 (1H, m), 7.27 (4H, m), 7.40 (3H, m), 7.55 (2H, m), 7.71 (2H, d, J=8.2 Hz), 7.83 (2H, d, J=8.2 Hz), 8.60 (1H, br s).

### Example 10-1

### Methyl N-(6-{4-[(Z)-2-phenylvinyl]phenyl}pyrimidin-4-yl)phenylalaninate

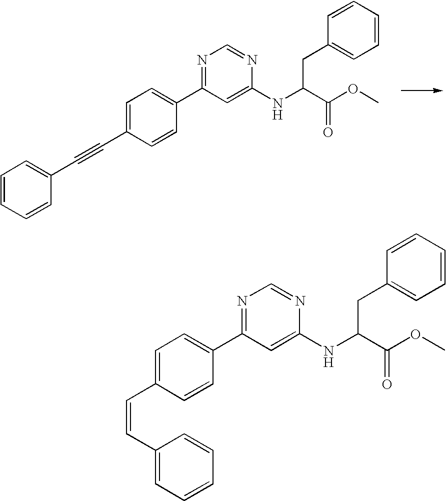

A mixture of methyl N-{6-[4-(phenylethynyl)phenyl]pyrimidin-4-yl}phenylalaninate (0.025 g, 0.06 mmol), palladium-barium sulfate (0.001 g), quinoline (0.01 mL) and methanol (2 mL) under a hydrogen atmosphere was stirred at room temperature for 2 hours. The resulting mixture was filtered through a Celite pad, and the filtrate was concentrated under reduced pressure. The residue was purified by preparative TLC (silica-gel, hexane:ethyl acetate, 7:3×5) to give methyl N-(6-{4-[(Z)-2-phenylvinyl]phenyl}pyrimidinyl) phenylalaninate (0.017 g, 69%) as colorless oil.

### N-(6-{4-[(Z)-2-Phenylvinyl]phenyl}pyrimidin-4-yl)phenylalanin

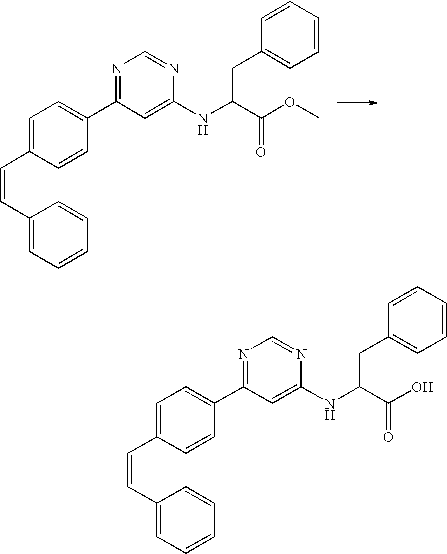

To a solution of methyl N-(6-{4-[(Z)-2-phenylvinyl]phenyl}pyrimidin-4-yl) phenyl alaninate (0.016 g, 0.04 mmol) in methanol (2 mL) was added 1M NaOH aqueous solution (0.5 mL) at room temperature, and the mixture was stirred for 3 hours. After the removal of methanol under reduced pressure, water was added to the residue. The solution was washed with diethyl ether, and the separated aqueous solution was acidified by 1N aqueous hydrochloric acid. The resulting precipitates were collected by filtration and dried under reduced pressure to give N-(6-{4-[(Z)-2-phenylvinyl]phenyl}pyrimidin-4-yl)phenylalanin (0.008 g, 53%) as slightly yellow solid.

Melting point: 217-220° C. (dec.)

Molecular weight: 421.503

Mass spectrometry: 422 (M+H)⁺

In vitro activity grade: A

¹H-NMR (500 MHz, MeOH-d4): δ 3.14 (1H, dd, J=8.8, 13.9 Hz), 3.42 (1H, dd, J=4.7, 13.9 Hz), 5.23 (1H, m), 6.69 (1H, d, J=12.0 Hz), 6.83 (1H, d, J=12.3 Hz), 7.01 (1H, br s), 7.24 (10H, m), 7.45 (2H, d, J=7.9 Hz), 7.64 (2H, d, J=8.2 Hz), 8.61 (1H, br s)

### Example 11-1

### Methyl N-[6-(4′-methoxybiphenyl-4-yl)pyrimidinyl]phenylalaninate

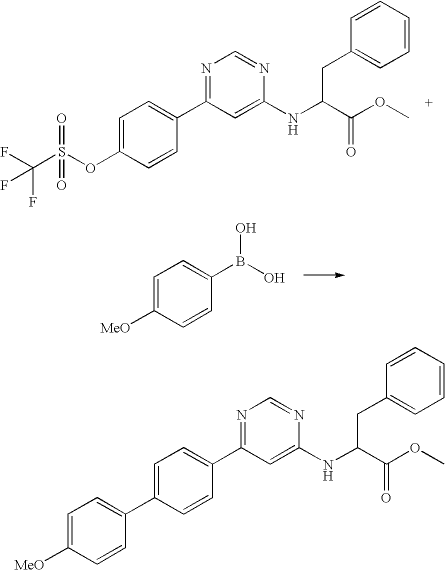

To a mixture of methyl N-[6-(4-{[(trifluoromethyl)sulfonyl]oxy}phenyl)pyrimidin-4-yl]phenylalaninate (0.060 g, 0.12 mmol), 4-methoxyphenylboronic acid (0.038 g, 0.25 mmol), potassium carbonate (0.052 g, 0.37 mmol) and benzene (0.4 mL) under an argon atmosphere was added tetrakis(triphenylphosphine)palladium (0.007 g, 0.01 mmol). The mixture was stirred at 85° C. overnight. After cooled to room temperature, the mixture was filtered through a Celite pad, and the filtrate was partitioned between ethyl acetate and water. The separated organic phase was washed with brine, dried over sodium sulfate, filtered and concentrated under reduced pressure. The crude product was purified by preparative TLC (silica-gel, hexane:ethyl acetate, 3:2) to give methyl N-[6-(4′-methoxybiphenyl-4-yl)pyrimidin-4-yl]-phenylalaninate (0.039 g, 72%) as yellow solid.

### N-[6-(4′-Methoxybiphenyl-4-yl)pyrimidin-4-yl]phenylalanine

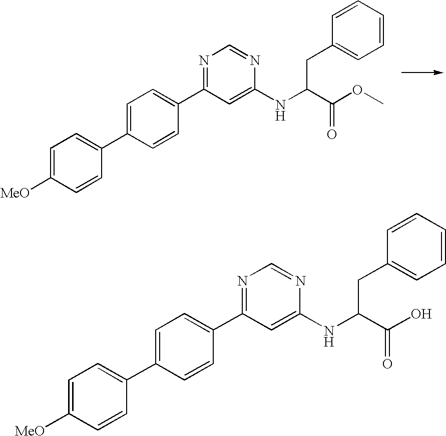

To a solution of methyl N-[6-(4′-methoxybiphenyl-4-yl)pyrimidinyl]phenylalaninate (0.038 g, 0.09 mmol) in methanol (1 mL) was added 1M NaOH aqueous solution (0.5 mL) at room temperature, and the mixture was stirred for 1 hour. After the removal of methanol under reduced pressure, water was added to the residue. The aqueous solution was washed with diethyl ether and neutralized by aqueous hydrochloric acid. The resulting precipitates were collected by filtration and dried under reduced pressure to give N-[6-(4′-methoxybiphenyl-4-yl)pyrimidin-4-yl]-phenylalanine (0.034 g, 92%) as slightly yellow solid

Melting point: 123-125° C.

Molecular weight: 425.492

Mass spectrometry: 426 (M+H)⁺

In vitro activity grade: A

¹H-NMR (500 MHz, DMSO-d6): δ 3.03 (1H, dd, J=9.5, 13.9 Hz), 3.22 (1H, m), 3.81 (3H, s), 4.82 (1H, m), 7.05 (2H, d, J=8.8 Hz), 7.11 (1H, br s), 7.20 (1H, m), 7.30 (4H, m), 7.70 (2H, d, J=8.8 Hz), 7.77 (21, d, J=8.5 Hz), 8.02 (2H, d, J=7.9 Hz), 8.53 (1H, br s).

### Examples 11-2 to 11-12

In the similar manners as described in Example 11-1 above, compounds in Examples 11-2 to 11-12 as shown in Table 11 were synthesized.

TABLE EXAMPLE 11

| | | | MASS | | |
|---|---|---|---|---|---|
| Ex. No | Structure | M.W. | (M + 1) | MP | In vitro |
| 11-2 | 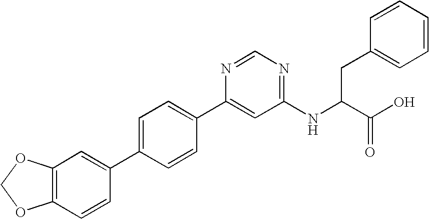 | 439.47 | 440 | 139-142Z | A |
| 11-3 | 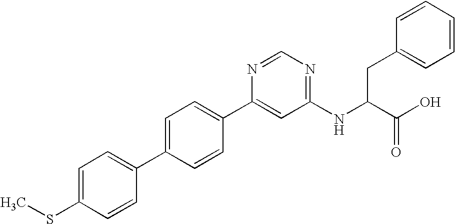 | 441.56 | 442 | 135-137Z | A |
| 11-4 | 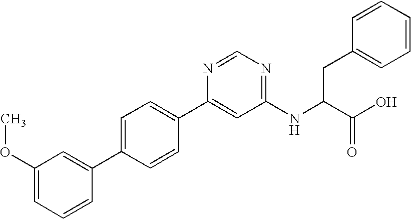 | 425.49 | 426 | 112-114Z | A |
| 11-5 | 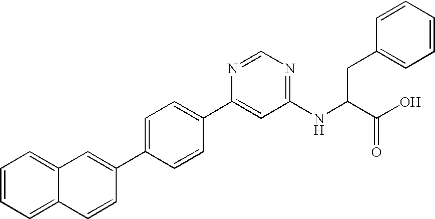 | 445.53 | 446 | 130-132Z | A |
| 11-6 | 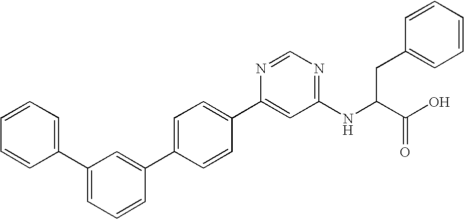 | 471.56 | 472 | 124-126Z | B |
| 11-7 | 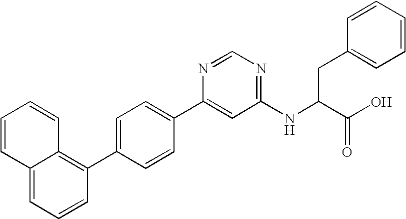 | 445.53 | 446 | 135-138Z | B |
| 11-8 | 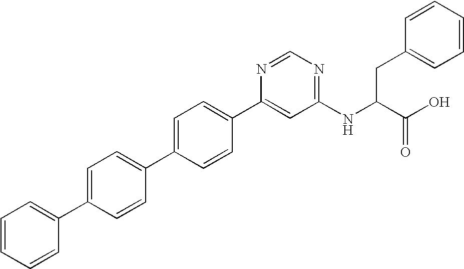 | 471.56 | 472 | 160Z | C |
| 11-9 | 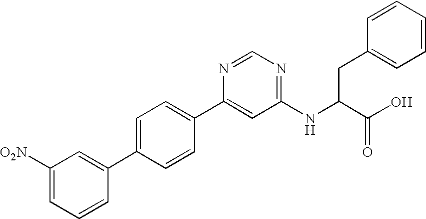 | 440.46 | 441 | 125-127Z | A |
| 11-10 | 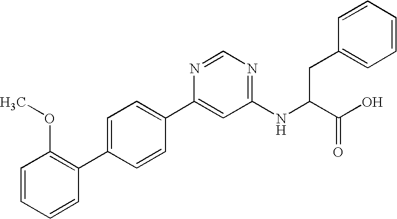 | 425.49 | 426 | 121-123Z | B |
| 11-11 | 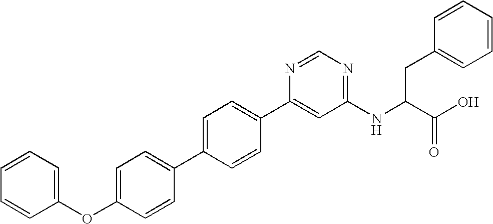 | 487.56 | 488 | 120-123Z | B |
| 11-12 | 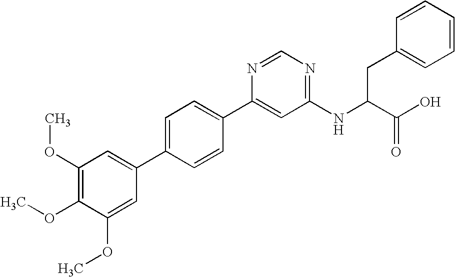 | 485.54 | 486 | 120-123z | B |

### Example 12-1

### Methyl N-(6-{4-[(4-cyanopyridin-2-yl)oxy]phenyl}pyrimidin-4-yl)phenylalaninate

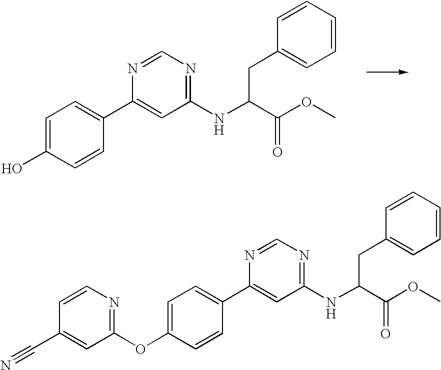

To a solution of methyl N-[6-(4-hydroxyphenyl)pyrimidin-4-yl]phenylalaninate (50 mg, 0.14 mmol) in dimethylsulfoxide (1.0 mL) were added 2-chloro-4-cyanopyridine (30 mg, 0.21 mmol) and potassium carbonate (30 mg, 0.21 mmol) and the mixture was stirred at 60° C. overnight. After cooled to room temperature, the mixture was poured into a mixture of ethyl acetate and water. The organic layer was separated and purified by preparative TLC (n-hexane/ethyl acetate, 1/1) to give methyl N-(6-{4-[(4-cyanopyridin-2-yl)oxy]phenyl}pyrimidin-4-yl)phenylalaninate (60.0 mg, 93%)as a gum.

### N-(6-{4-[(4-cyanopyridin-2-yl)oxy]phenyl}pyrimidin-4-yl)phenylalanine

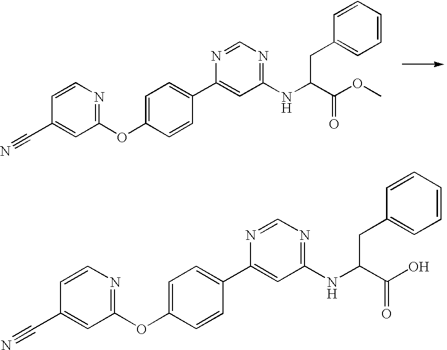

To an iced solution of N-(6-{4-[(4-cyanopyridin-2-yl)oxy]phenyl}pyrimidin-4-yl)phenylalaninate (60 mg, 0.13 mmol) in tetrahydrofuran (1.0 mL) was added 1N LiOH aqueous solution (0.16 mL, 0.16 mmol) and the mixture was stirred at room temperature overnight. The mixture was neutralized with 1N HCl (0.16 mL) and concentrated under reduced pressure. The resultant precipitate was collected by filtration, washed with water to give N-(6-{4[(4-cyanopyridin-2-yl)oxy]phenyl}-pyrimidin-4-yl)phenylalanine (37.1 mg, 64%) as a colorless powder.

Melting point: 139.5° C.

Molecular weight: 437.46

Mass spectrometry: 438 (M+H)⁺

In vitro activity grade: A

¹H-NMR (500 MHz, CD₃OD): δ 3.02 (1H, dd, J=13.7, 9.3 Hz), 3.21 (1H, dd, J=14.5, 4.6 Hz), 4.80 (1H, bs), 7.07 (1H, s), 7.17-7.21 (1H, bs), 7.27-7.33 (7H, m), 7.80 (1H, bs), 8.04 (2H, d, J=8.7, 2.4 Hz), 8.35 (1H, dd, J=8.7, 2.4 Hz), 8.50 (1H, s), 8.67 (1H, d, J=2.2 Hz), 12.77 (1H, bs).

### Examples 12-2 to 12-6

In the similar manners as described in Example 12-1 above, compounds in Examples 12-2 to 12-6 as shown in Table 12 were synthesized.

TABLE EXAMPLE 12

| | | | MASS | | |
|---|---|---|---|---|---|
| Ex No | Structure | M.W. | (M + 1) | MP | In vitro |
| 12-2 | 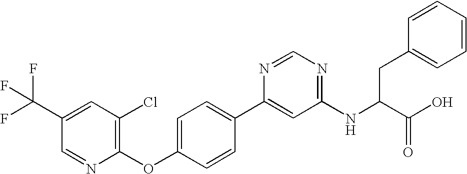 | 514.90 | 515 | 151 | C |
| 12-3 | 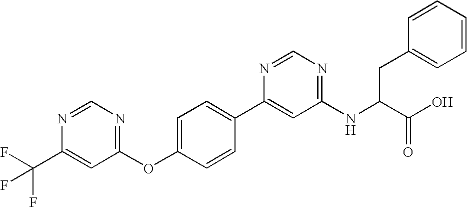 | 481.44 | 482 | 196 | B |
| 12-4 | 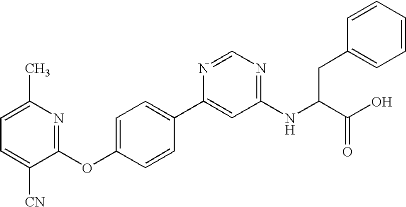 | 451.49 | 452 | 116Z | C |
| 12-5 | 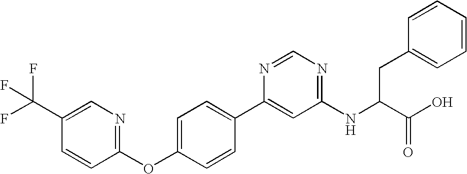 | 480.45 | 481 | 110Z | B |
| 12-6 | 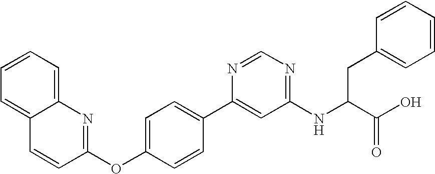 | 462.51 | 463 | 113Z | B |

### Example 13-1

### tert-Butyl (4-bromophenyl)carbamate

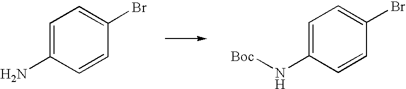

A solution of 4-bromoaniline (5.02 g, 29.18 mmol) and di-tert-butyl dicarbonate (7.64 g, 35.02 mmol) in toluene (150 mL) was stirred at 70° C. overnight. After the removal of toluene under reduced pressure, the residue was dissolved with ethyl acetate. The solution was washed with 0.1 M hydrochloric acid and brine, dried over sodium sulfate, filtered and concentrated under reduced pressure. The crude product was purified by recrystallization from hexane to give tert-butyl (4-bromophenyl)carbamate (6.56 g, 83%) as colorless needle crystals.

### tert-Butyl benzyl(4-bromophenyl)carbamate

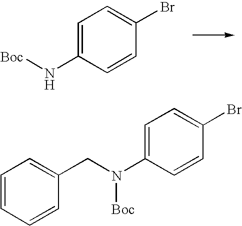

To a solution of tert-butyl (4-bromophenyl)carbamate (0.50 g, 1.84 mmol) and benzyl bromide (0.262 mL, 2.20 mmol) m tetrahydrofuran (20 mL) was added sodium hydride (60% in oil, 0.11 g, 2.76 mmol) at 0° C., and the mixture was stirred at room temperature for 1 hour and then at 60° C. for 4 hours. After cooled to room temperature, the reaction mixture was quenched with saturated ammonium chloride solution, and extracted with ethyl acetate. The separated organic phase was washed with brine, dried over sodium sulfate, filtered and concentrated under reduced pressure. The crude product was purified by column chromatography on silica-gel (hexane: ethyl acetate, 0.9:1) to give tert-butyl benzyl(4-bromophenyl)carbamate (0.68 g, 100%) as colorless oil.

### {4-[Benzyl(tert-butoxycarbonyl)amino]phenyl}boronic acid

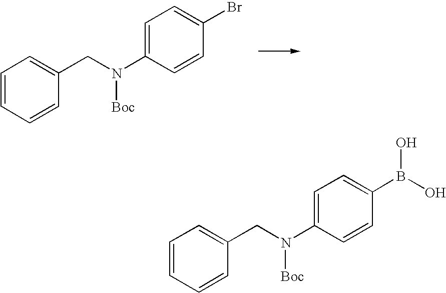

To a solution of tert-butyl benzyl(4-bromophenyl)carbamate (0.682 g, 1.88 mmol) in tetrahydrofuran (15 mL) was added dropwise n-butyl lithium (1.56 M in n-hexane, 1.45 mL, 2.26 mmol) at −78° C. After 10 minutes, trimethyl borate (0.27 mL, 2.45 mmol) was added dropwise. The reaction mixture was stirred for additional 30 minutes, and then allowed to warm to room temperature. The reaction was quenched with 1M hydrochloric acid (6 mL) and stirring was continued for 30 minutes. The mixture was extracted with ethyl acetate, and the extracts were dried over sodium sulfate, filtered, and concentrated under reduced pressure. The residue was purified by column chromatography on silica-gel (chloroform:methanol, 49:1) to give {4-[benzyl(tert-butoxycarbonyl)amino]phenyl}boronic acid (0.21 g, 35%) as colorless solid.

### Methyl N-(6-{4-[benzyl(tert-butoxycarbonyl)amino]phenyl}pyrimidin-4-yl)phenylalaninate

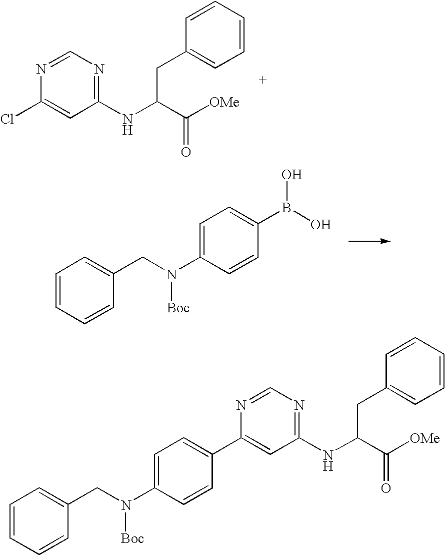

To a mixture of methyl N-(6-chloro-4-pyrimidinyl)phenylalaninate (0.12 g, 0.41 mmol), {4-[benzyl(tert-butoxycarbonyl)amino]phenyl}boronic acid (0.20 g, 0.61 mmol) and N,N-dimethylformamide (5 mL) under an argon atmosphere was added 2N sodium carbonate aqueous solution (0.41 mL, 0.82 mmol) followed by tetrakis(triphenylphosphine)palladium (0.024 g, 0.02 mmol). The mixture was stirred at 85° C. for 2 day. After cooled to room temperature, the mixture was partitioned between ethyl acetate and water. The separated organic phase was washed with brine, dried over sodium sulfate, filtered and concentrated under reduced pressure. The crude product was purified by column chromatography on silica-gel (hexane:ethyl acetate, 4:1) to give methyl N-(6-{4-[benzyl(tert-butoxycarbonyl)amino]phenyl}-pyrimidin-4-yl)phenylalaninate (0.03 g, 14%) as a colorless oil.

### N-(6-{4-[Benzyl(tert-butoxycarbonyl)amino]phenyl}pyrimidinyl)phenylalanine

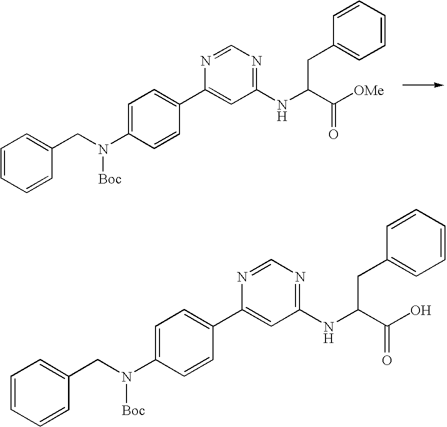

To a solution of methyl N-(6-{4-[benzyl(tert-butoxycarbonyl)amino]phenyl}-pyrimidin-4-yl)phenylalaninate (0.028 g, 0.05 mmol) in methanol (2 mL) was added 1M NaOH aqueous solution (0.5 mL) at room temperature, and the mixture was stirred for 3 hours. After the removal of methanol under reduced pressure, water was added to the residue. The solution was washed with diethyl ether and neutralized by aqueous hydrochloric acid. The resulting precipitates were collected by filtration and dried under reduced pressure to give N-(6-{4-[benzyl(tert-butoxycarbonyl)amino]-phenyl}pyrimidin-4-yl)phenylalanine (0.018 g, 66%) as slightly yellow solid.

### N-{6-[4-Benzylamino)phenyl]pyrimidin-4-yl}phenylalanine hydrochloride

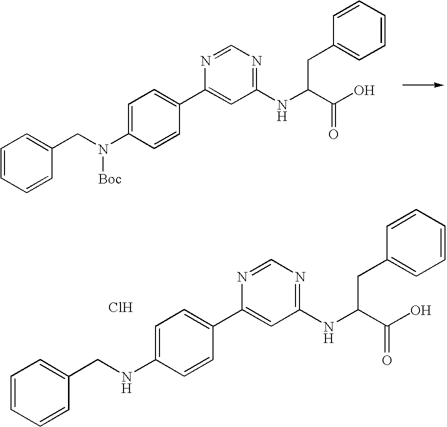

To a solution of N-(6-{4-[benzyl(tert-butoxycarbonyl)amino]phenyl}pyrimidin-4-yl)-phenylalanine (0.015 g, 0.03 mmol) in dioxane (2 mL) was added 4N hydrochloric acid dioxane solution (0.5 mL) at 0° C., and the mixture was stirred at room temperature overnight. The resulting precipitates were collected by filtration and dried under reduced pressure to give N-{6-[4-(enzylamino)phenyl]pyrimidin-4-yl}phenylalanine hydrochloride (0.012 g, 93%) as slightly yellow solid.

Melting point: 144-147° C. (dec.)

Molecular weight: 460.968

Mass spectrometry: 425 (M−HCl+H)⁺

In vitro activity grade: A

¹H-NMR (500 MHz, MeOH-d4): δ 3.11 (1H, dd, J=9.1, 14.2 Hz), 3.40 (1H, dd, J=5.014.2 Hz), 4.43 (2H, s), 5.20 (1H, dd, J=4.7, 9.1 Hz), 6.76 (2H, d, J=9.1 Hz), 6.86 (1H, s), 7.18-7.35 (10H, m), 7.55 (2H, d, J=8.8 Hz), 8.47 (1H, s)

### Example 14-1

### {4-[(tert-Butoxycarbonyl)amino]phenyl}boronic acid

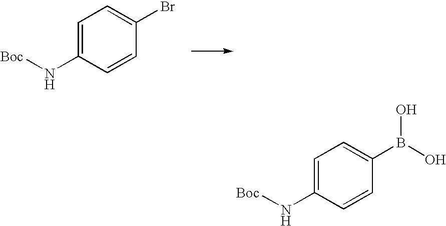

To a solution of tert-butyl (4-bromophenyl)carbamate (1.00 g, 3.67 mmol) in tetrahydrofuran (7 mL) was added dropwise a methyllithium solution (1.5 M in diethyl ether, 2.45 mL, 3.67 mmol) at 0° C. The mixture was stirred at 0° C. for 15 minutes and then cooled to −78° C., and n-butyl lithium (1.56 M in n-hexane, 1.45 mL, 2.26 mmol) was added dropwise. After the stirring for 1 hour, trimethyl borate (1.03 mL, 9.19 mmol) was added dropwise, and the reaction mixture was stirred for additional 45 minutes, and then at 0° C. for 1 hour. The reaction was treated with 5% hydrochloric acid for 15 minutes and NaCl was added to saturate the aqueous layer. The mixture was extracted with ethyl acetate, and the extracts were dried over sodium sulfate and concentrated in reduced pressure. The residue was purified by recrystallization from a mixture of hexane and ethyl acetate (4:1) to give {4-[(tertbutoxycarbonyl)amino]phenyl}boronic acid (0.48 g, 55%) as a colorless solid.

### Methyl N-(6-{[(tert-butoxycarbonyl)amino]phenyl}pyrimidin-4-yl)phenylalaninate

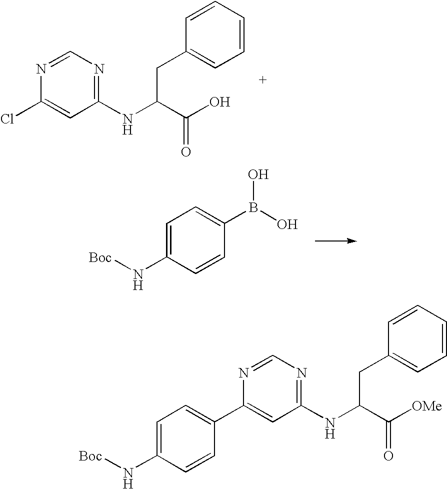

To a mixture of methyl N-(6-chloro-4-pyrimidinyl)phenylalaninate (0.49 g, 1.69 mmol), {[(tert-butoxycarbonyl)amino]phenyl}boronic acid (0.48 g, 2.02 mmol) and N,N-dimethylformamide (10 mL) under an argon atmosphere was added 2N sodium carbonate aqueous solution (1.69 mL, 3.37 mmol) followed by tetrakis(triphenylphosphine)palladium (0.097 g, 0.08 mmol). The mixture was stirred at 85° C. for 2 day. After cooled to room temperature, the mixture was partitioned between ethyl acetate and water. The separated organic phase was washed with brine, dried over sodium sulfate, filtered and concentrated under reduced pressure. The crude product was purified by column chromatography on silica-gel (hexane:ethyl acetate, 4:1) to give methyl N-(6-{4[(tertbutoxycarbonyl)amino]phenyl}pyrimidinyl)phenylalaninate (0.189 g, 25%) as colorless oil.

### Methyl N-[6-(4-aminophenyl)pyrimidin-4-yl]phenylalaninate hydrochloride

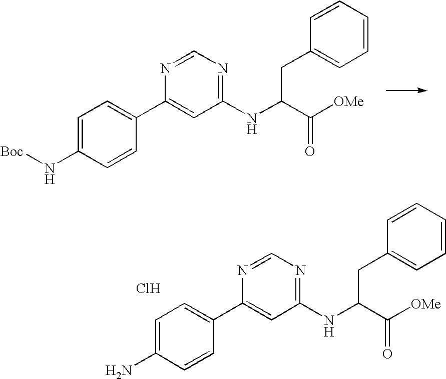

To a solution of methyl N-(6-{4-[(tert-butoxycarbonyl)amino]phenyl}pyrimidin-4-yl)phenylalaninate (0.187 g, 0.42 mmol) in dioxane (1 mL) was added 4N hydrochloric acid dioxane solution (2 mL) at 0° C., and the mixture was stirred at room temperature overnight. The resulting precipitates were collected by filtration, washed with diethyl ether and dried under reduced pressure to give 2-[6-(4-amino-phenyl)-pyrimidin-4-ylamino]-3-phenyl-propionic acid methyl ester hydrochloric acid (0.133 g, 83%) as slightly yellow solid.

### Methyl N-{6-[4-(benzoylamino)phenyl]pyrimidin-4-yl}phenylalaninate

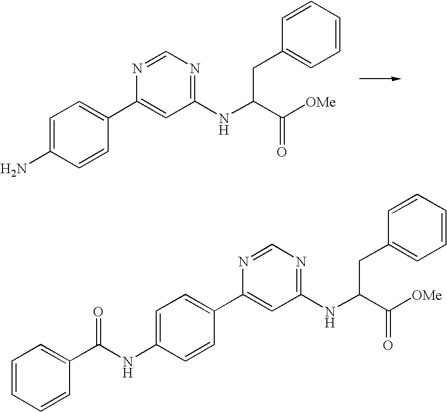

To a solution of methyl N-[6-(4-aminophenyl)pyrimidin-4-yl]phenylalaninate hydrochloride (0.020 g, 0.05 mmol) and N,N-diisopropylethylamine (0.027 mL, 0.16 mmol) in dichloromethane (1.5 mL) was added benzoyl chloride (0.007 mL, 0.06 mmol) at 0° C. After stirred at room temperature for 2 hours, the mixture was partitioned between dichloromethane and water. The separated organic phase was washed with brine, dried over sodium sulfate, filtered and concentrated under reduced pressure. The crude product was purified by preparative TLC (silica-gel, hexane:ethyl acetate, 1:1) to give methyl N-{6-[4-(benzoylamino)phenyl]pyrimidin-4-yl}phenylalaninate (0.021 g, 88%) as slightly yellow oil.

### N-{6-[4-(Benzoylamino)phenyl]pyrimidin-4-yl}phenylalanine

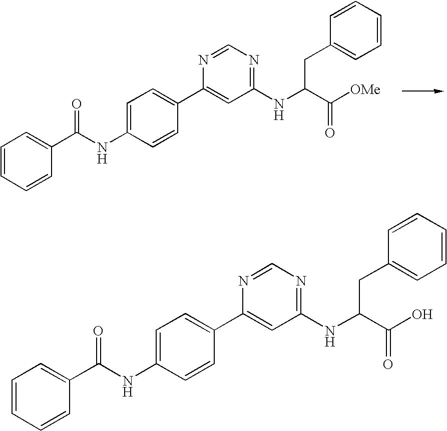

To a solution of methyl N-{6-[4-(benzoylamino)phenyl]pyrimidin-4-yl}phenylalaninate (0.020 g, 0.04 mmol) in methanol (1.5 mL) was added 1N NaOH aqueous solution (0.5 mL) at room temperature. The mixture was stirred for 30 minutes, and partitioned between diethyl ether and water. The separated aqueous phase was neutralized by aqueous hydrochloric acid. The resulting precipitates were collected by filtration and dried under reduced pressure to give N-{6-[4-(benzoylamino)phenyl]pyrimidin-4-yl}phenylalanine (0.012 g, 64%) as colorless solid.

Melting point: 250-252° C. (dec.)

Molecular weight: 438.490

Mass spectrometry: 439 (M+H)⁺

In vitro activity grade: B

¹H-NMR (500 MHz, DMSO-d6): δ 3.02 (1H, dd, J=9;8, 14.2 Hz), 3.21 (1H, dd, J=4.7, 14.2 Hz), 4.80 (1H, m), 7.04 (1H, br s), 7.20 (1H, m), 7.30 (4H, m), 7.55 (2H, t, J=7.3 Hz), 7.61 (1H, t, J=7.3 Hz), 7.75 (1H, br s), 7.92 (2H, d, J=8.8 Hz), 7.97 (4H, m), 8.48 (1H, br s), 10.43 (1H, s), 12.76 (1H, br s).

### Examples 14-2 and 14-3

In the similar manners as described in Example 14-1 above, compounds in Examples 14-2 and 14-3 as shown in Table 14 were synthesized.

TABLE EXAMPLE 14

| Ex. | | | MASS | | |
|---|---|---|---|---|---|
| No. | Structure | M.W. | (M + 1) | MP | In vitro |
| 14-2 | 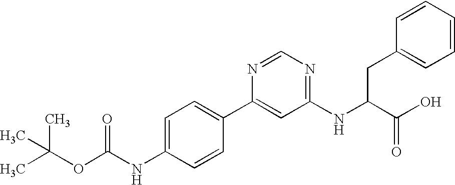 | 434.50 | 435 | 153-155Z | B |
| 14-3 | 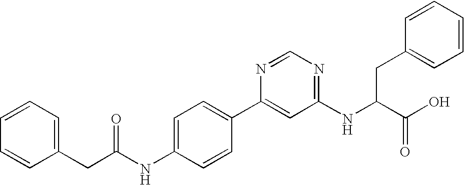 | 452.52 | 453 | 145-148Z | B |

### Example 15-1

### Methyl N-(6-{4-[(phenylsulfonyl)amino]phenyl}pyrimidin-4-yl)phenylalaninate

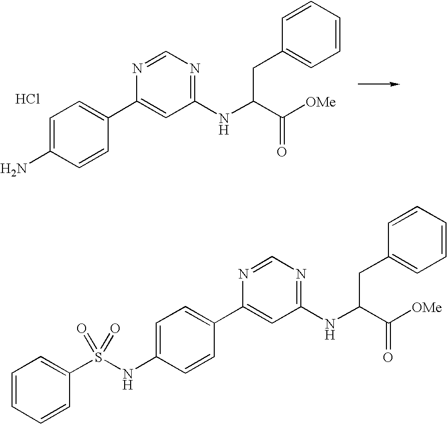

To a solution of methyl N-[6-(4-aminophenyl)pyrimidin-4-yl]phenylalaninate hydrochloride (0.015 g, 0.04 mmol) and N,N-diisopropylethylamine (0.02 mL, 0.12 mmol) in dichloromethane (1 mL) was added benzenesulfonyl chloride (0.006 mL, 0.05 mmol). The reaction mixture was stirred at room temperature for 2.5 hours, and partitioned between ethyl acetate and water. The separated organic phase was washed with brine, dried over sodium sulfate, filtered and concentrated under reduced pressure. The crude product was purified by preparative TLC (silica-gel, hexane:ethyl acetate, 7: 13) to give methyl N-(6-{4-[(phenylsulfonyl)amino]phenyl}pyrimidinyl)phenylalaninate (0.014 g, 71%) as slightly yellow oil.

### N-(6-{4-[(Phenylsulfonyl)amino]phenyl}pyrimidin-4-yl)phenylalanine

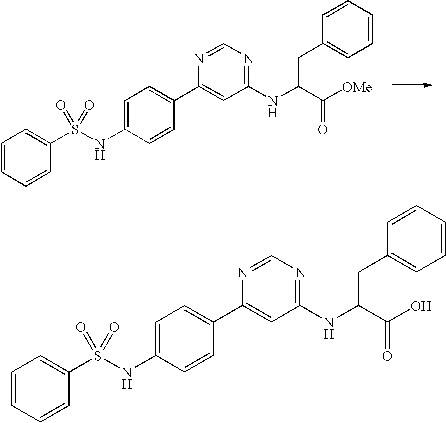

To a solution of methyl N-(6-{4-[(phenylsulfonyl)amino]phenyl}pyrimidin-4-yl)phenylalaninate (0.013 g, 0.03 mmol) in tetrahydrofuran (0.75 mL) and water (0.25 mL) was added lithium hydroxide monohydrate (0.0013 g, 0.03 mmol) at room temperature. The mixture was stirred for 2 hours and partitioned between diethyl ether and water. The separated aqueous phase was neutralized by 1N aqueous hydrochloric acid. The resulting precipitates were collected by filtration and dried under reduced pressure to give N-(6-{4-[(phenylsulfonyl)amino]phenyl}pyrimidinyl)phenylalanine (0.010 g, 79%) as yellow solid.

Melting point: 235-237° C. (dec.)

Molecular weight: 474.542

Mass spectrometry: 475 M+H)⁺

In vitro activity grade: C

¹H-NMR (500 MHz, MeOH-d4): δ 3.06 (1H, dd, J=8.5, 13.9 Hz), 3.33 (1H, m), 4.95 (1H, m), 6.84 (1H, br s), 7.16-7.25 (7H, m), 7.49 (2H, t, J=7.3 Hz), 7.57 (1H, t, J=7.3 Hz), 7.73 (2H, d, J=8.2 Hz), 7.81 (2H, d, J=8.5 Hz), 8.39 (1H, br s).

### Example 15-2

In the similar manners as described in Example 15-1 above, compound in Example 15-2 as shown in Table 15 was synthesized.

TABLE EXAMPLE 15

| Ex. | | | MASS | | |
|---|---|---|---|---|---|
| No. | Structure | M.W. | (M + 1) | MP | In vitro |
| 15-2 | 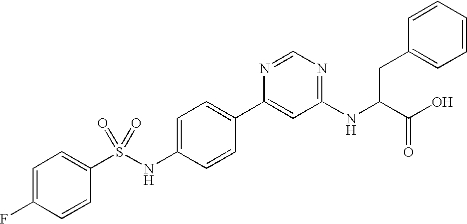 | 492.53 | 493 | 217-220Z | C |

### Example 16-1

### Methyl N-(6-{4-[(cyclopropylmethyl)amino]phenyl}pyrimidin-4-yl)phenylalaninate

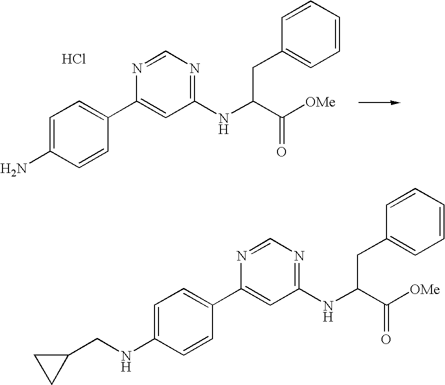

To a solution of methyl N-[6-(4-aminophenyl)pyrimidin-4-yl]phenylalaninate hydrochloride (0.02 g, 0.05 mmol) and cyclopropanecarboxyaldehyde (0.006 mL, 0.08 mmol) in methanol was added sodium cyanoborohydride (0.004 g, 0.06 mmol). The mixture was stirred at room temperature overnight and partitioned between ethyl acetate and water. The separated organic phase was washed with brine, dried over sodium sulfate, filtered and concentrated under reduced pressure. The crude product was purified by preparative TLC (silica-gel, hexane:ethyl acetate, 1:1) to give methyl N-(6-{4-[(cyclopropylmethyl)amino]phenyl}pyrimidin-4-yl)phenylalaninate (0.01 g, 48%) as yellow oil.

### N-(6-{4-[(Cyclopropylmethyl)amino]phenyl}pyrimidin-4-yl)phenylalanine

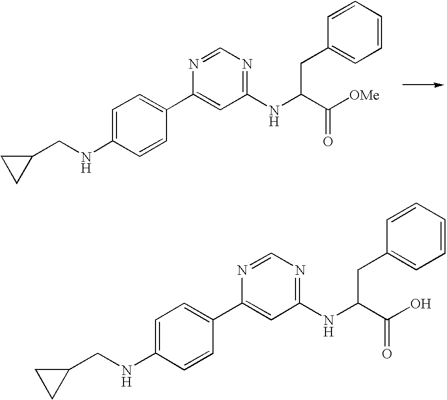

To a solution of methyl N-(6-{4-[(cyclopropylmethyl)amino]phenyl}pyrimidin-4-yl)phenylalaninate (0.010 g, 0.02 mmol) in methanol (1 mL) was added 1N NaOH aqueous solution (0.5 mL) at room temperature, and the mixture was stirred for 30 minutes. After the removal of methanol under reduced pressure, water was added to the residue. The solution was washed with diethyl ether and neutralized by 1N aqueous hydrochloric acid. The resulting precipitates were collected by filtration and dried under reduced pressure to give N-(6-{4-[(cyclopropylmethyl)amino]phenyl}-pyrimidinyl)phenylalanine (0.006 g, 65%) as yellow solid.

Melting point: 135-138° C. (dec.)

Molecular weight: 388.473

Mass spectrometry: 389 (M+H)⁺

In vitro activity grade: B

¹H-NMR (500 MHz, MeOH-d4): δ 0.26 (2H, m), 0.55 (2H, m), 1010(1H, m), 3.03 (2H, d, J=6.6 Hz), 3.07 (1H, dd, J=9.1, 13.6 Hz), 3.38 (1H, m), 5.06 (1H, m), 6.72 (2H, d, J=8.8 Hz), 6.83 (1H, br s), 7.17 (1H, m), 7.26 (4H, m), 7.59 (2H, d, J=8.8 Hz), 8.38 (1H, br s)

### Example 17-1

### Methyl N-[6-(4-formylphenyl)pyrimidin-4-yl]phenylalaninate

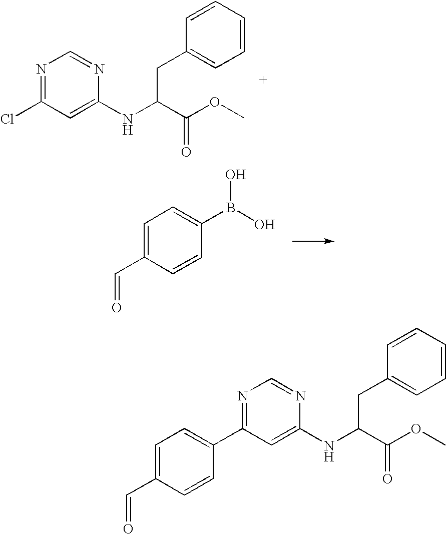

To a mixture of methyl N-(6-chloropyrimidin-4-yl)phenylalaninate (300 mg, 1.03 mmol) and tetrakis(triphenylphosphine)palladium (0) (59 mg, 0.05 mmol) in benzene (10 mL) was added 2M sodium carbonate solution (2.1 mL) and followed by 4-formylphenylboronic acid (231 mg, 1.54 mmol) in ethanol (4.5 mL). The reaction mixture was stirred for 2.5 hours at 90° C. After cooling this, this mixture was diluted with water and extracted with ethyl acetate. The organic phase was washed with brine, dried over magnesium sulfate, filtered and concentrated under reduced pressure. The residue was purified by column chromatography on silica gel (hexane/-ethyl acetate, 3:1) to give methyl N-[6-(4-formylphenyl)pyrimidin-4-yl]phenylalaninate (346 mg, 93%).

### Methyl N-(6-{4-[hydroxy(phenyl)methyl]phenyl}pyrimidin-4-yl)phenylalaninate

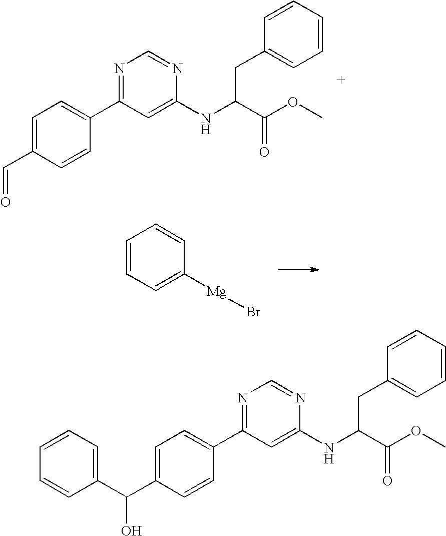

To a solution of methyl N-[6-(4-formylphenyl)pyrimidin-4-yl]phenylalaninate (140 mg, 0.39 mmol) in tetrahydrofuran (3 mL) was added dropwise a phenylmagnesium bromide solution (1M, 0.78 mL, 0.78 mmol, in tetrahydrofuran) at −78° C. The mixture was stirred at −78° C. for 2 hours, and then quenched with saturated ammonium chloride solution, and extracted with ethyl acetate. The organic phase was washed with water and brine, dried over magnesium sulfate and concentrated under reduced pressure. The residue was purified by preparative TLC (hexane/ethyl acetate, 2:1) to give methyl N-(6-{4-[hydroxy(phenyl)methyl]phenyl}-pyrimidin-4-yl)phenylalaninate (117 mg, 69%).

### Methyl N-[6-(4-benzoylphenyl)pyrimidin-4-yl]phenylalaninate

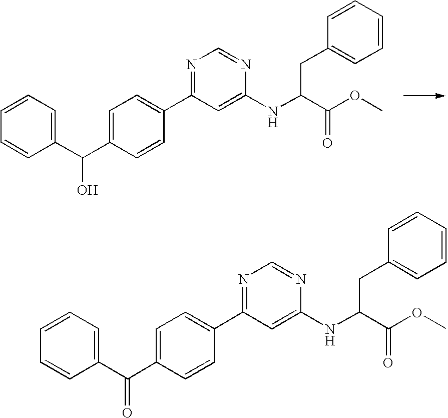

To a mixture of methyl N-(6-{[hydroxy(phenyl)methyl]phenyl}pyrimidin-4-yl)phenylalaninate (59 mg, 0.13 mmol), N-methylmorpholine N-oxide (47 mg, 0.40 mmol) and molecular sieve 4A (50 mg) in dichloromethane (2 mL) was added tetrapropylammonium perruthenate (TPAP, 9.4 mg, 0.03 mmol). The reaction mixture was stirred at room temperature for 18 hours. This mixture was diluted with water and extracted with ethyl acetate. The organic phase was washed with water and brine, dried over magnesium sulfate, filtered and concentrated under reduced pressure. The residue was purified by preparative TLC (hexane/ethyl acetate, 2:1) to give methyl N-[6-(4-benzoylphenyl)pyrimidin-4-yl]phenylalaninate (40 mg, 68%).

### N-[6-(4-Benzoylphenyl)pyrimidin-4-yl]phenylalanine

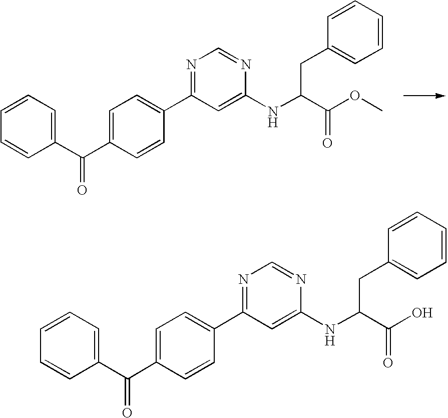

A mixture of methyl N-[6-(4-benzoylphenyl)pyrimidin-4-yl]phenylalaninate (15 mg, 0.03 mmol) in methanol (0.2 mL) and tetrahydrofuran (0.2 mL) was added dropwise 1N aqueous sodium hydroxide (0.1 mL, 0.1 mmol). This mixture was stirred at room temperature for 3 hours, then acidified with 1N hydrochloric acid, and concentrated under reduced pressure. The residual precipitate was collected by filtration, washed with diisopropylether, and dried under reduced pressure to give N-[6-(4-benzoylphenyl) pyrimidin-4-yl]phenylalanine (12 mg, 83%) as a white solid.

Melting point: 109-111° C.

Molecular weight: 423.47

In vitro activity grade: A

Mass spectrometry: 424 (M+H)⁺

¹H-NMR (500 MHz, DMSO-d6): δ 3.03 (1H, dd, J=9.0, 14.0 Hz), 3.16-3.24 (1H, m), 4.82 (1H, m), 7.16-7.19 (2H, m), 7.25-7.30 (4H, m), 7.59 (2H, t, J=7.6 Hz), 7.70 (1H, t, J=7.2 Hz), 7.77 (2H, d, J=7.9 Hz), 7.86 (2H, d, J=8.2 Hz), 7.88 (1H, br.s), 8.13 (2H, d, J=7.6 Hz), 8.54 (1H, s), 12.8 (1H, br.s).

### Examples 17-2 to 17-5

In the similar manners as described in Example 17-1 above, compounds in Examples 17-2 to 17-5 as shown in Table 17 were synthesized.

TABLE EXAMPLE 17

| Ex. | | | MASS | | In |
|---|---|---|---|---|---|
| No | Structure | M.W. | (M + 1) | MP | vitro |
| 17-2 | 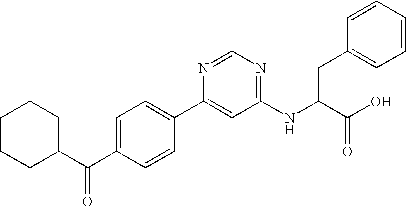 | 429.52 | 430 | 116-119 | B |
| 17-3 | 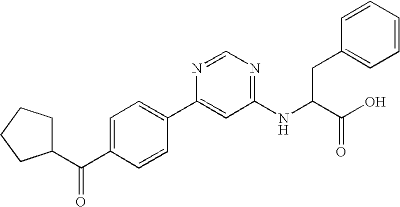 | 415.50 | 416 | 114-116 | A |
| 17-4 | 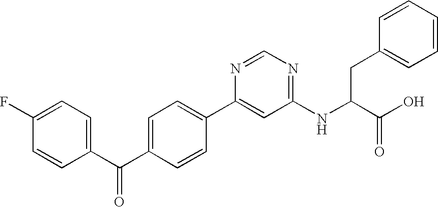 | 441.47 | 442 | 115-117 | A |
| 17-5 | 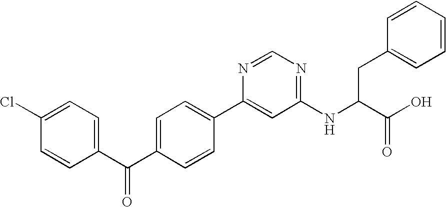 | 457.92 | 458 | 123-126 | A |

### Example 18-1

### Methyl N-[6-(4-benzylphenyl)pyrimidin-4-yl]phenylalaninate

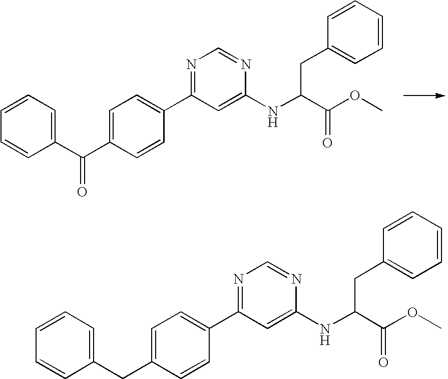

To a mixture of methyl N-[6-(4-benzoylphenyl)pyrimidin-4-yl]phenylalaninate (30 mg, 0.07 mmol) in trifluoroacetic acid (0.5 mL) at 0° C. was added dropwise triethylsilane (0.03 mL, 0.21 mmol). The reaction was stirred at room temperature for 18 hours. The mixture was concentrated under reduced pressure. The residue was purified by preparative TLC (hexane/ethyl acetate, 2:1) to give methyl N-[6-(4-benzylphenyl)pyrimidin-4-yl]phenylalaninate (26 mg, 90%).

### N-[6-(4-Benzylphenyl)pyrimidin-4-yl]phenylalanine

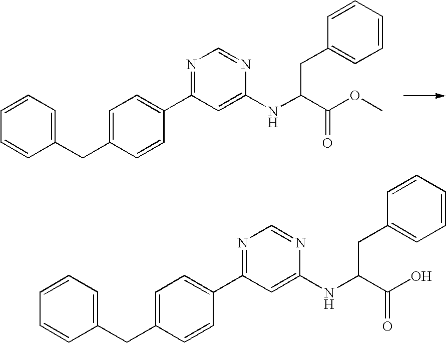

A mixture of methyl N-[6-(4-benzylphenyl)pyrimidin-4-yl]phenylalaninate (19 mg, 0.04 mmol) in methanol (0.2 mL) and tetrahydrofuran (0.2 mL) was added dropwise 1N aqueous sodium hydroxide solution (0.1 mL, 0.1 mmol). This mixture was stirred for 3 hours at room temperature, then acidified with 1N hydrochloric acid and concentrated under reduced pressure. The residual precipitate was collected by filtration, washed with diisopropylether, and dried under reduced pressure to give N-[6-(4-benzylphenyl)pyrimidin-4-yl]phenylalanine (15 mg, 82%) as a white solid.

Melting point: 116-118° C.

Molecular weight: 409.49

Mass spectrometry: 410 (M+H)⁺

¹H-NMR (500 MHz, DMSO-d6): δ 3.02 (1H, dd, J=9.4, 13.9 Hz), 3.19-3.23 (1H, m), 4.01 (1H, s), 4.82 (1H, m), 7.03 (1H, s), 7.16-7.33 (10H, m), 7.59 (2H, d, J=8.2 Hz), 7.85 (2H, d, J=7.6 Hz), 8.00 (1H, br.s), 8.52 (1H, s), 12.8 (1H, br.s).

### Examples 18-2 and 18-3

In the similar manners as described in Example 18-1 above, compounds in Examples 18-2 and 18-3 as shown in Table 18 were synthesized.

TABLE
Example 18

| Ex. | | | MASS | | |
|---|---|---|---|---|---|
| No. | Structure | M.W. | (M + 1) | MP | In vitro |
| 18-2 | 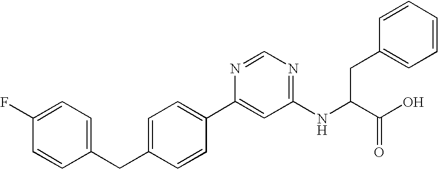 | 427,48 | 428 | 98-101 | B |
| 18-3 | 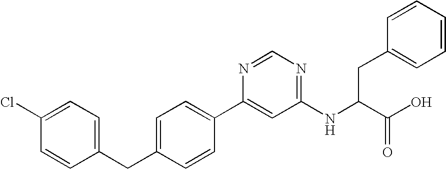 | 443,94 | 444 | 105-107 | B |

### Example 19-1

### Methyl N-{6-[4-(anilinomethyl)phenyl]pyrimidin-4-yl}phenylalaninate

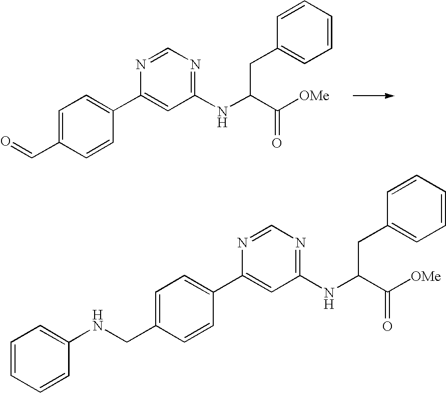

A mixture of methyl N-[6-(4-formylphenyl)pyrimidin-4-yl]phenylalaninate (0.05 g, 0.14 mmol), aniline (0.015 mL, 0.17 mmol) and sodium sulfate (0.098 g, 0.69 mmol) in acetic acid (1.5 mL) was stirred at room temperature for 1 hour, and then sodium triacetoxyborohydride (0.044 g, 0.21 mmol) was added. After the stirring for 30 minutes, the mixture was filtered trough a Celite pad, and the filtrate was concentrated under reduced pressure. The residue was partitioned between chloroform and water. The separated organic phase was washed with brine, dried over sodium sulfate, filtered and concentrated under reduced pressure. The crude product was purified by preparative TLC (hexane:ethyl acetate, 7:13) to give methyl N-{6-[4-(anilinomethyl)phenyl]pyrimidin-4-yl}phenylalaninate (0.061 g, 100%) as yellow oil.

### N-{6-[4-(Anilinomethyl)phenyl]pyrimidinyl}phenylalanine

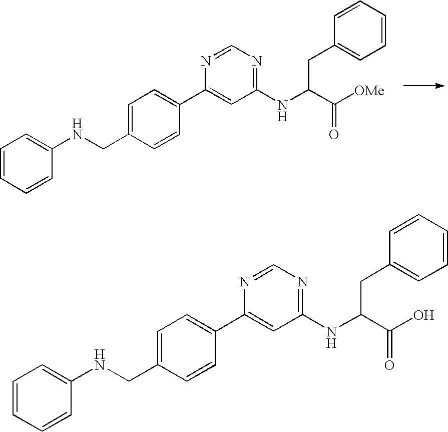

To a solution of methyl N-{6-[4-(anilinomethyl)phenyl]pyrimidinyl}phenylalaninate (0.058 g, 0.13 mmol) in methanol (2 mL) was added 1M NaOH aqueous solution (0.5 mL) at room temperature, and the mixture was stirred for 3 hours. After the removal of methanol under reduced pressure, water was added to the residue. The aqueous solution was washed with diethyl ether and neutralized by aqueous hydrochloric acid. The resulting precipitates were collected by filtration and dried under reduced pressure to give N-{6-[4-(anilinomethyl)phenyl]pyrimidin-4-yl}phenylalanine (0.035 g, 62%) as slightly yellow solid.

Melting point: 115-118° C. (dec.)

Molecular weight: 424.507

Mass spectrometry. 425 (M+H)⁺

In vitro activity grade: A

¹H-NMR (500 MHz, MeOH-d4): δ 3.09 (1H, dd, J=8.5, 13.9 Hz), 3.34 (11, dd, J=4.1, 13.9 Hz), 4.39 (2H, s), 5.01 (1H, m), 6.58 (1H, t, J=7.6 Hz), 6.61 (2H, d, J=8.5 Hz), 6.92 (1H, br s), 7.05 (2H, t, J=7.6 Hz), 7.17 (1H, m), 7.25 (4H, m), 7.51 (2H, d, J=8.2 Hz), 7.79 (2H, d, J=8.2 Hz), 8.45 (1H, br s).

### Examples 19-2 to 19-4

In the similar manners as described in Example 19-1 above, compounds in Examples 19-2 to 19-4 as shown in Table 19 were synthesized.

TABLE EXAMPLE 19

| Ex. | | | MASS | | |
|---|---|---|---|---|---|
| No. | Structure | M.W. | (M + 1) | MP | In vitro |
| 19-2 | 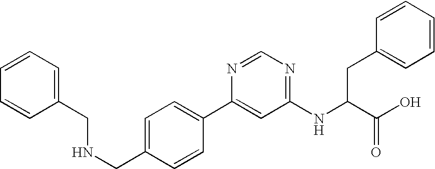 | 438.53 | 439 | 173-176Z | B |
| 19-3 | 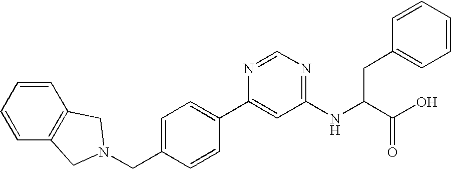 | 450.55 | 451 | 145Z | B |
| 19-4 | 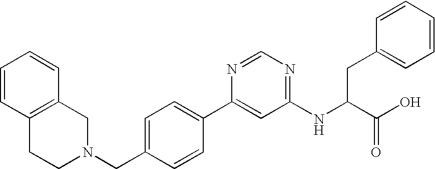 | 464.57 | 465 | 148-150 | C |

### Example 20-1

### Methyl N-{6-[4-(hydroxymethyl)phenyl]pyrimidin-4-yl}phenylalaninate

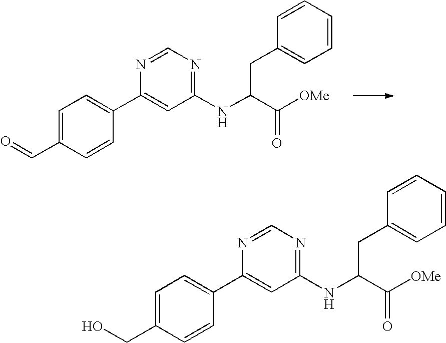

To a solution of methyl N-[6-(4-formylphenyl)pyrimidin-4-yl]phenylalaninate (0.06 g, 0.17 mmol) in methanol (1.5 mL) was added sodium borohydride (0.009 g, 0.25 mmol) at 0° C. The mixture was stirred at room temperature for 2 hours and quenched with water. After removal of solvent under reduced pressure, the residue was partitioned between ethyl acetate and water. The separated organic phase was washed with brine, dried over sodium sulfate, filtered and concentrated under reduced pressure. The crude product was purified by column chromatography on silica-gel (hexane:ethyl acetate, 3:7) to give methyl N-{6-[4-hydroxymethyl)phenyl]pyrimidin-4-yl}phenylalaninate (0.04 g, 67%) as colorless solid.

### Methyl N-{6-[4-(phenoxymethyl)phenyl]pyrimidin-4-yl}phenylalaninate

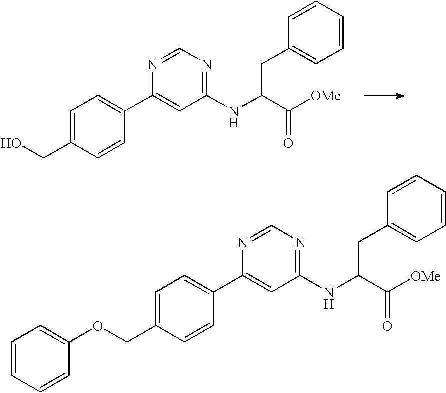

To a cold (0° C.) solution of methyl N-{6-[4-(hydroxymethyl)phenyl]pyrimidin-4-yl}-phenylalaninate (0.029 g, 0.08 mmol), phenol (0.0075 g, 0.08 mmol) and triphenylphosphine (0.021 g, 0.08 mmol) in dichloromethane (1 mL) was added diethyl azodicarboxylate (40% in toluene, 0.031 mL, 0.08 mmol). The reaction mixture was stirred at room temperature for 3 hours and concentrated under reduced pressure. The resulting crude product was purified by preparative TLC (hexane:ethyl acetate, 1:1) to give methyl N-{6-[4-(phenoxymethyl)phenyl]pyrimidin-4-yl}phenylalaninate (0.027 g, 77%) as yellow oil.

### N-{6-[4-(Phenoxymethyl)phenyl]pyrimidin-4-yl}phenylalanine

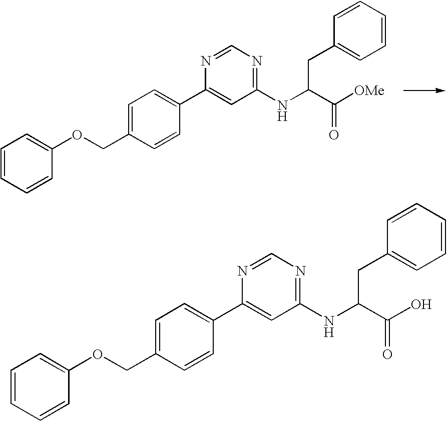

To a solution of methyl N-{6-[4-(phenoxymethyl)phenyl]pyrimidin-4-yl}phenylalaninate (0.020 g, 0.05 mmol) in methanol (2 mL) was added 1N NaOH aqueous solution (0.5 mL) at room temperature, and the mixture was stirred for 3 hours. After the removal of methanol under reduced pressure, water was added to the residue. The solution was washed with diethyl ether and neutralized by 1N aqueous hydrochloric acid. The resulting precipitates were collected by filtration and dried under reduced pressure to give N-{6-[4-phenoxymethyl)phenyl]pyrimidin-4-yl}phenylalanine (0.009 g, 45%) as colorless solid.

Melting point: 207-210° C. (dec.)

Molecular weight: 425.49

Mass spectrometry: 426 (M+H)⁺

In vitro activity grade: A

¹H-NMR (500 MHz, MeOH-d4): δ 3.14 (1H, dd, J=9.1, 13.9 Hz), 3.43 (1H, dd, J=4.4, 14.2 Hz), 5.21 (2H, s), 5.23 (1H, m), 6.94 (1H, tt, J=1.0, 7.6 Hz), 6.99 (2H, dd, J=1.0, 8.5 Hz), 7.04 (1H, br s), 7.21 (1H, m), 7.27 (6H, m), 7.69 (2H, d, J=8.2 Hz), 7.80 (2H, d, J=8.2 Hz), 8.63 (1H, br s).

### Example 20-2

In the similar manners as described in Example 20-1 above, compounds in Example 20-2 as shown in Table 20 was synthesized.

TABLE EXAMPLE 20

| Ex. | | | MASS | | |
|---|---|---|---|---|---|
| No. | Structure | M.W. | (M + 1) | MP | In vitro |
| 20-2 | 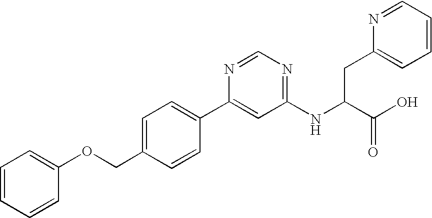 | 426.48 | 427 | 81-84 | A |

### Example 21-1

### Methyl N-(6-{4-[(E)-(phenoxyimino)methyl]phenyl}pyrimidin-4-yl)phenylalaninate

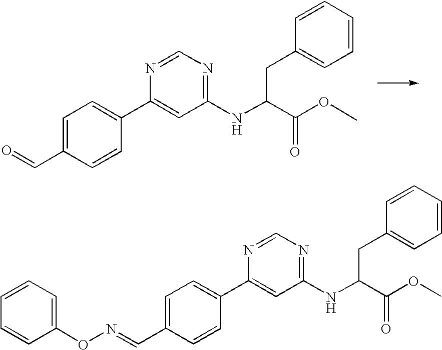

A mixture of methyl N-[6-(4-formylphenyl)pyrimidin-4-yl]phenylalaninate (30.0 mg, 0.08 mmol), O-phenylhydroxylamine hydrochloride (18.1 mg, 0.12 mmol) and sodium acetate (102.1 mg, 1.25 mmol) and methanol (2.0 mL) was stirred at room temperature overnight. The mixture was partitioned between ethyl acetate and water. The organic layer was separated, washed with brine, dried over sodium sulfate, filtered and concentrated under reduced pressure. The residue was purified by preparative TLC (n-hexane/ethyl acetate, 2/1) to give methyl N-(6-{4-[(E)-(phenoxyimino)methyl]phenyl}pyrimidin-4-yl)phenylalaninate (36.0 mg, 96%) as a gum.

### N-(6-{4-[(E)-(phenoxyimino)methyl]phenyl}pyrimidin-4-yl)phenylalanine

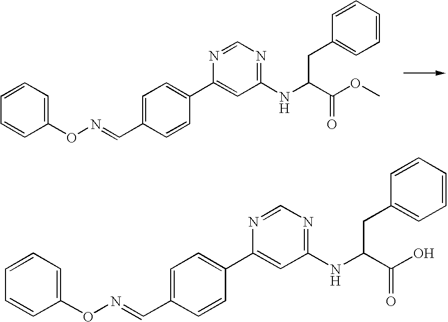

To an iced solution of methyl N-(6-{4-[(E)-(phenoxyimino)methyl]phenyl}-pyrimidinyl-4-yl)phenyl-alaninate (36.0 mg, 0.08 mmol) in tetrahydrofuran (1.0 mL) was added 1N LiOH aqueous solution (0.12 mL, 0.12 mmol) and the mixture was stirred at room temperature overnight. After neutralized with 1N HCl (0.12 mL), the mixture was partitioned between ethyl acetate and water. The organic layer was separated, washed with brine, dried over sodium sulfate, filtered, and concentrated under reduced pressure. The residue was crystallized from ethyl ether, washed with diisopropyl ether to give N-(6-{4-[(E)-(phenoxyimino)methyl]phenyl}pyrimidin-4-yl)phenylalanine (27.0 mg, 77%) as an ivory powder.

Melting point: 152.8° C.

Molecular weight: 438.49

Mass spectrometry: 439 (M+H)⁺

In vitro activity grade: A

¹H-NMR (500 MHz, DMSO-d6): δ 3.09 (1H, dd, J=13.2, 10.1 Hz), 3.21(1H, m), 4.78 (1H, m), 7.08 (1H, t, J=7.3 Hz), 7.13 (1H, bs), 7.20 (1H, bs), 7.27-7.33 (7H, m), 7.39 (2H, t, J=8.4 Hz), 7.82 (1H, bs), 7.91 (2H, d, J=8.5 Hz), 8.09 (2H, d, J=7.3 Hz), 8.51 (1H, s), 12.81 (1H, bs).

### Example 22-1

### 4,6-Dichloropyrimidine-5-carbaldehyde

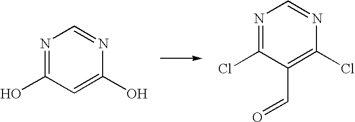

A mixture of phosphorus oxychloride (20 mL, 0.22 mol) and N,N-dimethylformamide (6.4 mL) was stirred at 0° C. for 1 hour. 4,6-Dichloropyrimidine (5.00 g, 44.6 mmol) was added to the reaction mixture, which was then stirred for 3 hours at 120° C. After cooled to room temperature, the reaction mixture was concentrated under reduced pressure. The residue was diluted with ice-water and extracted with ether. The separated organic phase was washed with saturated sodium hydrogen carbonate solution and brine, dried over sodium sulfate, filtered and concentrated under reduced pressure. The residual solid was triturated with hexane to give 4,6-dichloro-pyrimidine-5-carbaldehyde (4.73 g, 60%).

### Methyl N-(6-chloro-5-formylpyrimidin-4-yl)phenylalaninate

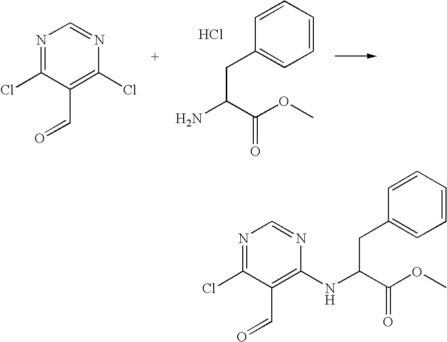

A mixture of 4,6-dichloropyrimidine-5-carbaldehyde (50 mg, 0.28 mmol) and methyl phenylalaninate hydrochloride (61 mg, 0.28 mmol), N,N-diisopropylethylamine (0.10 mL, 0.57 mmol) and methanol (1.5 mL) was stirred at 50° C. for 18 hours. After cooled to room temperature, the reaction mixture was concentrated under reduced pressure. The residue was purified by preparative TLC (hexane/ethyl acetate, 2:1) to give methyl N-(6-chloro-5-formylpyrimidin-4-yl)phenylalaninate (63 mg, 70%).

### Methyl N-{6-[4-(benzyloxy)phenyl]-5-formylpyrimidin-4-yl}phenylalaninate

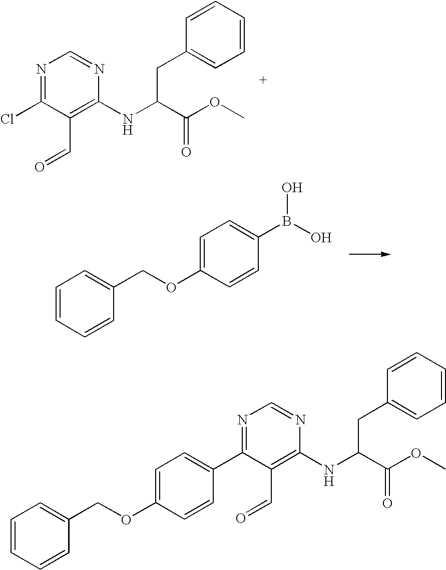

To a mixture of methyl N-(6-chloro-5-formylpyrimidin-4-yl)phenylalaninate (300 mg, 0.94 mmol), tetrakis(triphenylphosphine)palladium (0) (54 mg, 0.05 mmol) and potassium carbonate (389 mg, 2.81 mmol) in benzene (3 mL) was added (4-benzyloxyphenyl)boronic acid (321 mg, 1.41 mmol). The reaction mixture was stirred at 80° C. for 19 hours. After cooled to room temperature, this mixture was filtered through a pad of celite. The filtrate was concentrated under reduced pressure. The residue was purified by preparative TLC (hexane/ethyl acetate, 2:1) to give methyl N-{6-[4-(benzyloxy)phenyl]-5-formylpyrimidin-4-yl}phenylalaninate (432 mg, 99%).

### N-{6-[4-(Benzyloxy)phenyl)-5-formylpyrimidin-4-yl}phenylalanine

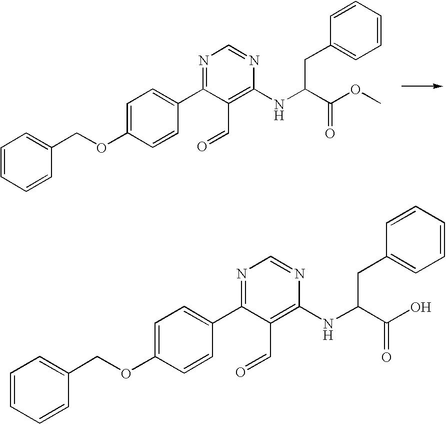

A mixture of methyl N-{6-[4-(benzyloxy)phenyl]-5-formylpyrimidin-4-yl}phenylalaninate (30 mg, 0.06 mmol) in methanol (0.3 mL) and tetrahydrofuran (0.3 mL) was added dropwise 1N aqueous sodium hydroxide (0.1 mL, 0.1 mmol). The mixture was stirred for 3 hours at room temperature, then acidified with 1N hydrochloric acid and concentrated under reduced pressure. The residual precipitate was collected by filtration, washed with diisopropylether and ethyl acetate, and dried under reduced pressure to give N-{6-[4-(benzyloxy)phenyl]-5-formylpyrimidin-4-yl}phenylalanine (10 mg, 34%) as a white solid.

Melting point: >300° C.

Molecular weight: 453.5

Mass spectrometry: 454 (M+H)⁺

In vitro activity grade: B

¹H-NMR (500 MHz, DMSO-d6): δ 3.09 (1H, dd, J=5.0, 13.2 Hz), 3.24 (1H, dd, J 5.7, 13.2 Hz), 4.40 (1H, s), 5.19 (2H, s), 7.06-7.17 (7H, m), 7.34 (1H, t, J=7.3 Hz), 7.41 (2H, t, J=7.0 Hz), 7.48 (2H, d, J=7.3 Hz), 7.56 (2H, d, J=8.8 Hz), 8.55 (1H, s), 9.58 (1H, d, J=6.4 Hz), 9.74 (1H, s).

### Example 23-1

### Methyl N-[6-[4-(benzyloxy)phenyl]-5-(hydroxymethyl)pyrimidin-4-yl]phenylalaninate

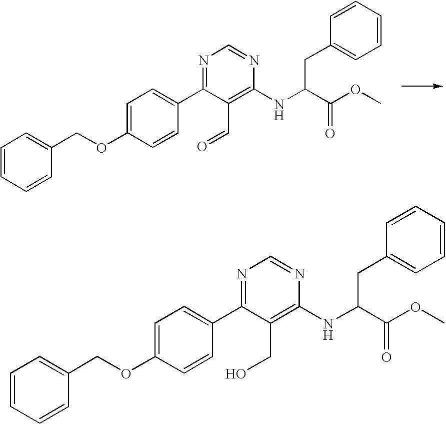

To a solution of methyl N-{6-[4-(benzyloxy)phenyl]-5-formylpyrimidin-4-yl}phenylalaninate (100 mg, 0.21 mmol) in methanol (2 mL) was added sodium borohydride (8.9 mg, 0.24 mmol). This mixture was stirred for 2 hours at room temperature. The reaction mixture was quenched with saturated ammonium chloride solution and extracted with ethyl acetate. The organic phase was washed with water and brine, dried over magnesium sulfate and concentrated under reduced pressure. The residue was purified by preparative TLC (hexane/ethyl acetate, 3:2) to give methyl N-[6-[4-(benzyloxy)phenyl]-5-(hydroxymethyl)pyrimidin-4-yl]phenylalaninate (81 mg, 81%).

### N-[6-[4-(Benzyloxy)phenyl]-5-(hydroxymethyl)pyrimidin-4-yl]phenylalanine

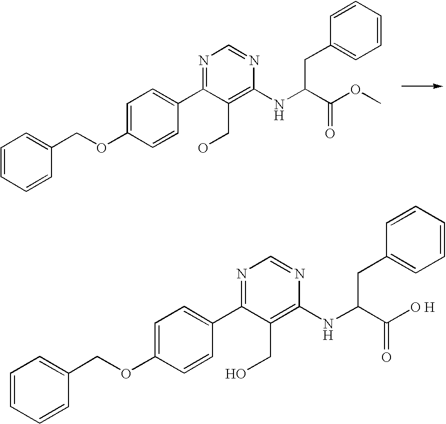

A mixture of methyl N-[6-[4-(benzyloxy)phenyl]-5-(hydroxymethyl)pyrimidin-4-yl]-phenylalaninate (22 mg, 0.05 mmol) in methanol (0.3 mL) and tetrahydrofuran (0.3 mL) was added dropwise 1N aqueous sodium hydroxide (0.1 mL, 0.1 mmol).

This mixture was stirred at room temperature for 3 hours, then acidified with 1N hydrochloric acid, and concentrated under reduced pressure. The residual precipitate was collected by filtration, washed with diisopropylether, and dried under reduced pressure to give N-[6-[4-(benzyloxy)phenyl]-5-(hydroxymethyl)pyrimidin-4-yl]phenylalanine (15 mg, 70%) as a white solid.

Melting point: 114-117° C.

Molecular weight: 455.51

Mass spectrometry: 456 (M+H)⁺

In vitro activity grade: B

¹H-NMR (500 MHz, DMSO-d6): δ 3.14 (1H, dd, J=7.6, 13.8 Hz), 3.24 (1H, d, J 5.0, 13.8 Hz), 4.34 (1H, d, J=12.0 Hz), 4.43 (1H, d, J=12.0 Hz), 4.91 (1H, s), 5.18 (2H, s), 5.44 (1H, s), 7.11 (2H, d, J=8.8 Hz), 7.18-7.30 (5H, m), 7.34 (1H, t, J=7.4 Hz), 7.41 (2H, t, J=7.2 Hz), 7.48 (2H, d, J=7.0 Hz), 7.54 (2H, d, J=8.5 Hz), 8.46 (1H, s), 12.9 (1H, br.s).

### Example 24-1

### N-(3-bromophenyl)phenylalanine

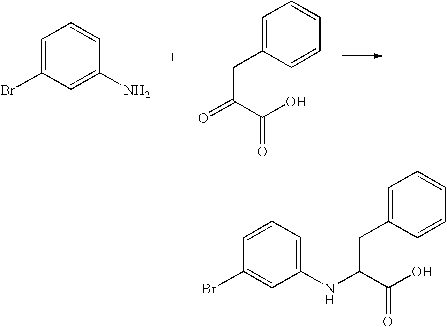

A mixture of 3-bromoaniline (3.50 g, 20.4 mmol), phenylpyruvic acid (6.68 g, 40.7 mmol) and sodium sulfate (28.9 g, 0.203 mol) and acetic acid (20 mL) was stirred for 1 h, and then sodium triacetoxyborohydride (4.74 g, 22.4 mmol) was added. The mixture was stirred at room temperature for 3 days, diluted with water, and extracted with chloroform. The organic phase was washed with water and brine, dried over sodium sulfate and concentrated under reduced pressure. The residue was purified by column chromatography on silica gel (chloroform/methanol, 30:1) to give N-(3-bromophenyl)phenylalanine (1.88 g, 29%).

### Methyl N-(3-bromophenyl)phenylalaninate

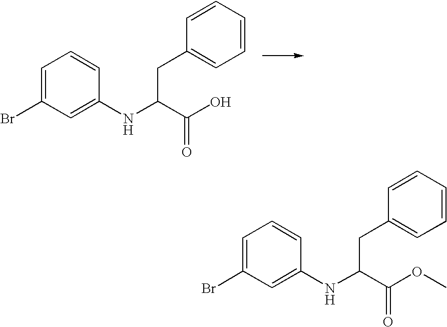

To a solution of 2-(3-bromo-phenylamino)-3-phenyl-propionic acid (1.50 g, 4.68 mmol) in ether (20 mL) was added a solution of diazomethane in ether. This mixture was stirred at room temperature for 30 minutes, and concentrated under reduced pressure. The residue was purified by column chromatography on silica gel (hexane/ethyl acetate, 30:1) to give methyl N-(3-bromophenyl)phenylalaninate (1.40 g, 89%).

### Methyl N-[4′-(benzyloxy)biphenyl-3-yl]phenylalaninate

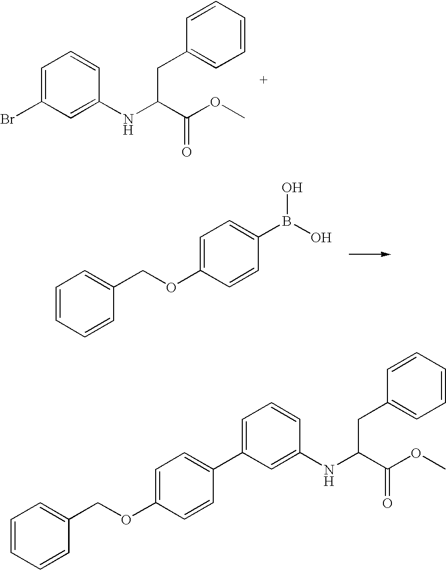

To a mixture of methyl N-(3-bromophenyl)phenylalaninate (500 mg, 1.50 mmol), tetrakis(triphenylphosphine)palladium (0) (86 mg, 0.07 mmol) and cesium fluoride (909 mg, 5.98 mmol) and 1,2-dimethoxyethane (5 mL) was added portionwise (4-benzyloxyphenyl)boronic acid (682 mg, 2.99 mmol). This mixture was stirred at 100° C. for 18 hours. After cooled to room temperature, the reaction mixture was diluted with water and extracted with chloroform. The separated organic phase was washed with water and brine, dried over magnesium sulfate, filtered and concentrated under reduced pressure. The residue was purified by column chromatography on silica-gel (hexane/ethyl acetate, 20:1) to give methyl N-[4′-(benzyloxy)biphenyl-3-yl]phenylalaninate (620 mg, 95%).

### N-[4′-(Benzyloxy)biphenyl-3-yl]phenylalanine

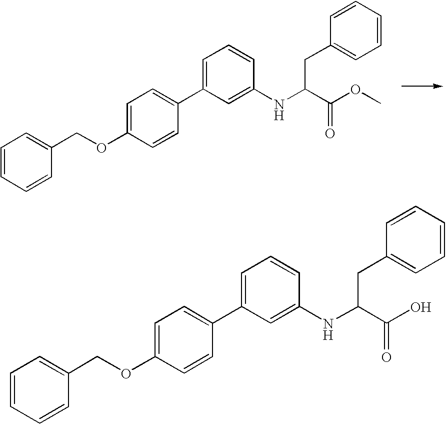

A mixture of methyl N-[4′-(benzyloxy)biphenyl-3-yl]phenylalaninate (31 mg, 0.07 mmol), methanol (0.5 mL) and tetrahydrofuran (0.5 mL) was added dropwise 1N aqueous sodium hydroxide (0.3 mL, 0.3 mmol). This mixture was stirred at room temperature for 2 hours, then acidified with 1N hydrochloric acid and extracted with chloroform The organic phase was dried over magnesium sulfate, filtered and concentrated under reduced pressure. The residue was purified by preparative TLC (chloroform/methanol 30:1) to give N-[4′-(benzyloxy)biphenyl-3-yl]phenylalanine (25 mg, 83%) as a pale yellow solid.

Melting point: 152-154° C.

Molecular weight: 423.51

Mass spectrometry: 424 (M+H)⁺

In vitro activity grade: A

¹H-NMR (500 MHz, DMSO-d6): δ 2.97 (1H, dd, J=7.8, 12.8 Hz), 3.09 (1H, dd, J=5.3, 12.8 Hz), 4.14 (1H, s), 5.14 (3H, s), 6.51 (1H, d, J=8.2 Hz), 6.75 (1H, s), 7.06 (2H, d, J=8.5 Hz), 7.09 (1H, d, J=7.8 Hz), 7.18 (1H, t, J=7.0 Hz), 7.26 (2H, t, J=7.6 Hz), 7.30 (2H, d, J=7.3 Hz), 7.34 (1H, d, J=7.3 Hz), 7.40 (2H, d, J=7.3 Hz), 7.46 (2H, d, J=8.2 Hz), 7.47 (1H, d, J=8.9 Hz).

### Example 24-2

### Methyl N-(4′-hydroxybiphenyl-3-yl)phenylalaninate

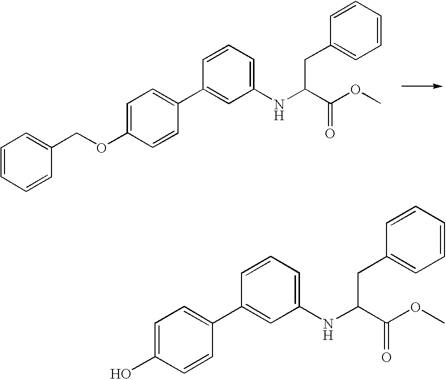

A suspension of methyl N-[4′-(benzyloxy)biphenyl-3-yl]phenylalaninate (212 mg, 0.48 mmol) and 10% palladium on activated carbon (5 mg) in tetrahydrofuran (2 mL) and ethyl acetate (2 mL) under a hydrogen atmosphere was stirred for 18 hours. The reaction mixture was filtered through a pad of celite. The filtrate was concentrated under reduced pressure. The residue was purified by preparative TLC (hexane/ethyl acetate, 3:1) to give methyl N-(4′-hydroxybiphenyl-3-yl)phenylalaninate (113 mg, 67%).

### Methyl N-{4′-[(3-methoxybenzyl)oxy]biphenyl-3-yl}phenylalaninate

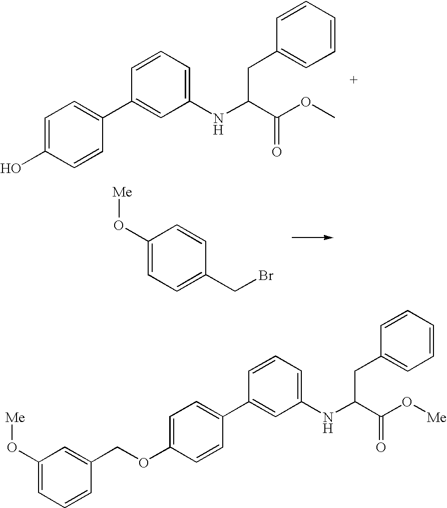

To a stirred solution of methyl N-(4′-hydroxybiphenyl-3-yl)phenylalaninate (20 mg, 0.06 mmol) and 3-methoxybenzyl bromide (14 mg, 0.07 mmol) in acetone (1 mL) was added potassium carbonate (8.8 mg, 0.06 mmol). The reaction mixture was stirred at room temperature for 17 hours. This mixture was purified by preparative TLC (hexane/ethyl acetate, 5:1) to give methyl N-{4′-[(3-methoxybenzyl)oxy]-biphenyl-3-yl}phenylalaninate (23 mg, 86%).

### N-{4′-[(3-Methoxybenzyl)oxy]biphenyl-3-yl}phenylalanine

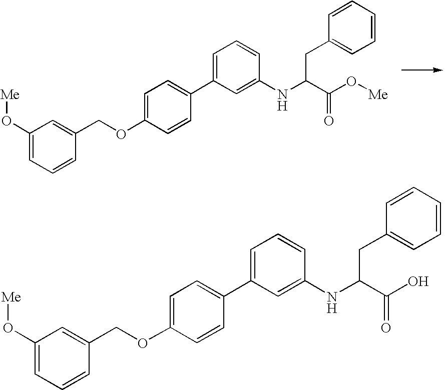

A mixture of methyl N-{4′-[(3-methoxybenzyl)oxy]biphenyl-3-yl}phenylalaninate (21 mg, 0.05 mmol) in methanol (0.3 mL) and tetrahydrofuran (0.3 mL) was added dropwise 1N aqueous sodium hydroxide (0.3 mL, 0.3 mmol). This mixture was stirred at room temperature for 2 hours, then acidified with 1N hydrochloric acid, and concentrated under reduced pressure. The residual precipitate was collected by filtration, washed with water, and dried under reduced pressure to give N-{4′-[(3-methoxybenzyl)oxy]biphenyl-3-yl}phenylalanine (18 mg, 81%) as a white solid.

Melting point: 159-162° C.

Molecular weight: 453.54

Mass spectrometry: 454 (M+H)⁺

In vitro activity grade: A

¹H-NMR (500 MHz, DMSO-d6): δ 2.99 (1H, dd, J=8.2, 13.6 Hz), 3.09 (1H, dd, J=5.7, 13.6 Hz), 3.76 (3H, s), 4.21 (1H, s), 5.11 (2H, s), 6.53 (1H, d, J=8.7 Hz), 6.78 (2H, s), 6.89 (2H, d, J=7.6 Hz), 7.00-7.12 (5H, m), 7.20 (1H, t, J=7.0 Hz), 7.26-7.32 (5H, m), 7.47 (1H, d, J=8.8 Hz).

### Examples 24-3 to 24-7

In the similar manners as described in Examples 24-1 and 24-2 above, compounds in Examples 24-3 to 24-7 as shown in Table 24 were synthesized.

TABLE EXAMPLE 24

| Ex. | | | MASS | | |
|---|---|---|---|---|---|
| No. | Structure | M.W. | (M + 1) | MP | In vitro |
| 24-3 | 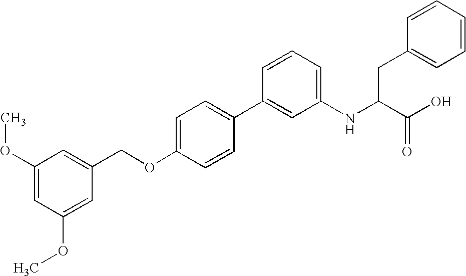 | 483.57 | 484 | 166-168 | A |
| 24-4 | 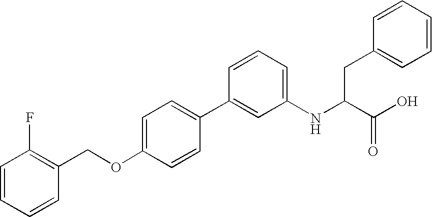 | 441.51 | 442 | 158-161 | A |
| 24-5 | 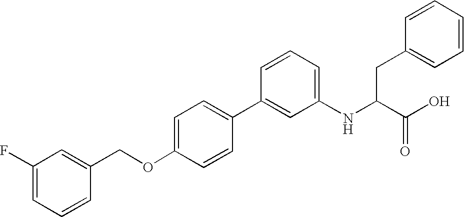 | 441.51 | 442 | 167-169 | A |
| 24-6 | 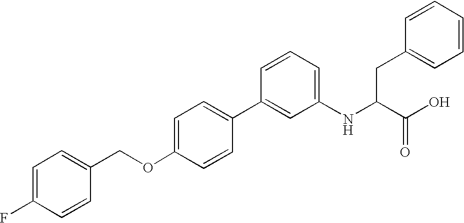 | 441.51 | 442 | 175-178 | B |
| 24-7 | 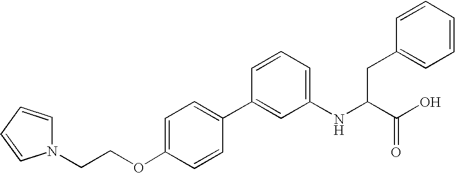 | 426.52 | 427 | 182-185 | A |

### Example 25-1

### 2-(Benzyloxy)-5-bromopyridine

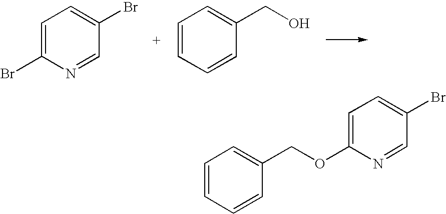

A mixture of 2,5-dibromopyridine (20 g, 84.4 mmol), dibenzo-18-crown-6 (1.5 g, 4.2 mmol); benzyl alcohol (11.9 g, 11.4 mL, 109.8 mmol), potassium hydroxyde (11.4 g, 202.6 mmol) and toluene (200 mL) was atirred at reflux with a Dean-Stark apparatus for 1.5 hours. After removal of solvent in reduced pressure, the residue was diluted with water, and extracted with chloroform. The separated organic phase was dried over magnesium sulfate, filtered and concentrated under reduced pressure. The crude product was purified by column chromatography on silica-gel, (hexane:ethyl acetate, 98:2) followed by recrystallization from hexane, to give 2-(benzyloxy)-5-bromopyridine (20.6 g, 92%) as a colorless solid.

### 2-(Benzyloxy)-5-(tributylstannyl)pyridine

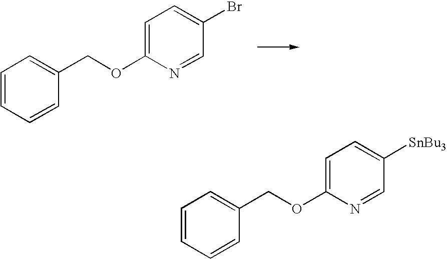

To a solution of 2-(benzyloxy)-5-bromopyridine (10.0 g, 37.9 mmol) in diethyl ether (200 mL) was added n-butyllithium (1.56 M in n-hexane, 29.1 mL, 45.4 mmol) at −78° C. After the stirring at −78° C. for 30 minutes, tributyltin chloride was added. The reaction mixture was stirred at −78° C. for further 1 hour, and quenched with aqueous potassium fluoride slution. The solution was extracted with ethyl ether, and the extracts were washed with brine, dried over sodium sulfate, filtered and concentrated under reduced pressure. The residue was purified by column chromatography on silica-gel (hexane:ethyl acetate, 98:2) to give 2-(benzyloxy)-5-(tributylstannyl)pyridine (15.4 g, 86%) as a colorless oil.

### Methyl N-{6-[6-(benzyloxy)pyridin-3-yl]pyrimidin-4-yl}phenylalaninate

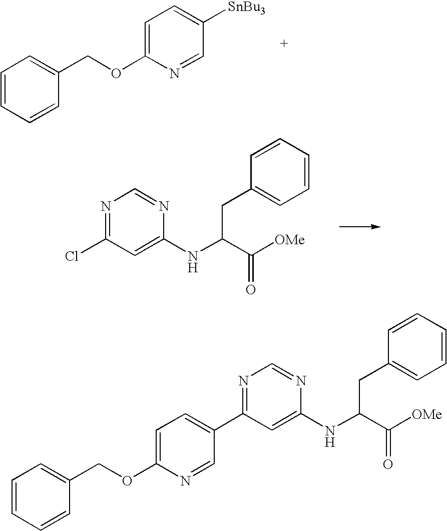

A mixture of methyl N-(6-chloro-4-pyrimidinyl)phenylalaninate (0.100 g, 0.34 mmol), 2-(benzyloxy)-5-(tributylstannyl)pyridine (0.195 g, 0.41 mmol), tetrakis(triphenylphosphine)palladium (0.024 g, 0.02 mmol) in N,N-dimethylformamide (2 mL) was stirred at 100° C. overnight. After cooled to room temperature, the reaction mixture was quenched with aqueous potassium fluoride solution and stirred at room temperature for 3 hours. The resulting precipitates were removed by filteration, and the filterate was extracted with ethyl acetate. The separated organic phase was washed with brine, dried over sodium sulfate, filtered and concentrated under reduced pressure. The residue was purified by column chromatography on silica-gel (hexane:ethyl acetate, 8:2) to give methyl N-{6-[6-(benzyloxy)pyridin-3-yl]-pyrimidin-4-yl}phenylalaninate (0.104 g, 69%) as a colorless oil.

### N-{6-[6-(Benzyloxy)pyridin-3-yl]pyrimidin-4-yl}phenylalanine

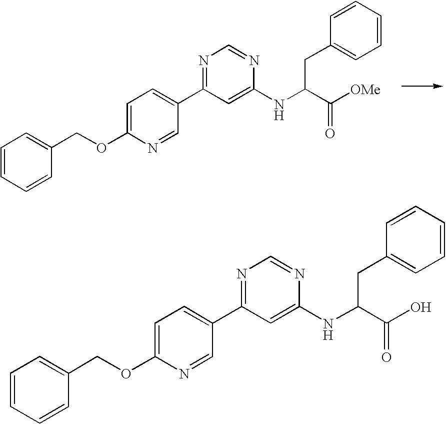

To a solution of methyl N-{6-[6-(benzyloxy)pyridin-3-yl]pyrimidin-4-yl}phenyl alaninate (0.100 g, 0.23 mmol) in methanol (2 mL) was added 1N NaOH aqueous solution (0.5 mL) at room temperature for 0.1 hour. After the removal of methanol under reduced pressure, water was added to the residue. The aqueous solution was washed with diethyl ether, acidified by aqueous hydrochloric acid, and extracted with ethyl acetate. The separated organic phase was washed with brine, dried over sodium sulfate, filtered and concentrated under reduced pressure. The crude product was purified by recrystallization from a mixture of iso-propanol and diisopropylether to give N-{6-[6-(benzyloxy)pyridin-3-yl]pyrimidin-4-yl}phenylalanine (0.060 g, 62%) as colorless solid.

Melting point: 130-133° C.

Molecular weight: 426.48

Mass spectrometry: 427 (M+H)⁺

In vitro activity grade: A

¹H-NMR (500 MHz, DMSO-d6): δ 3.02(1H, dd, J=9.5, 13.9 Hz), 3.20 (1H, dd, J=4.4, 13.9 Hz), 4.79 (1H, m), 5.42 (2H, s), 6.99 (1H, d, J=8.8 Hz), 7.01 (1H br s), 7.20 (1H, m), 7.29 (4H, m), 7.33 (1H, t, J=7.3 Hz), 7.39 (2H, t, J=7.3 Hz), 7.47 (2H, d, J=7.3 Hz), 7.72 (1H, br s), 8.25 (1H, d, J=5.7 Hz), 8.47 (1H, s), 8.78 (1H, br s), 12.75 (1H, br s).

### Examples 25-2 and 25-3

In the similar manners as described in Example 25-1 above, compounds in Examples 25-2 and 25-3 as shown in Table 25 were synthesized.

TABLE EXAMPLE 25

| Ex. | | | MASS | | |
|---|---|---|---|---|---|
| No. | Structure | M.W. | (M + 1) | MP | In vitro |
| 25-2 | 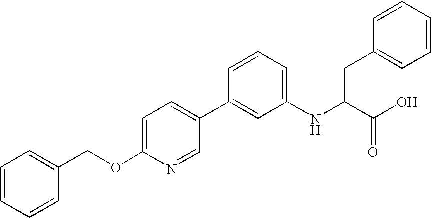 | 424.50 | 425 | 150-153 | B |
| 25-3 | 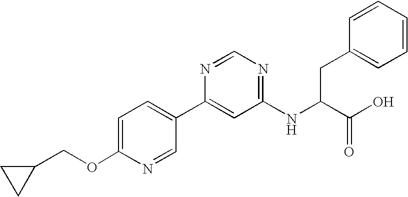 | 390.45 | 391 | 113-114 | A |

### Example 26-1

### N-{6-[4-(Benzyloxy)phenyl]pyrimidinyl}-N-{[tert-butyl(dimethyl)silyl]oxy}-phenylalaninamide

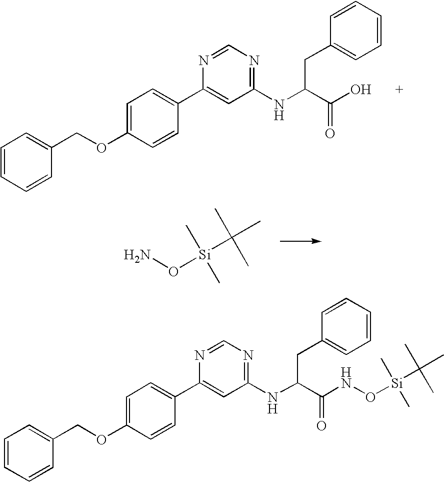

To a cold (0° C.) mixture of N-{6-[4-(benzyloxy)phenyl]pyrimidinyl}phenylalanine (0.059 g, 0.14 mmol), O-(tert-butyldimethylsilyl)hydroxylamine (0.031 g, 0.21 mmol), 1-hydroxybenzotriazole hydrate (0.028 g, 0.21 mmol) and DMF (3 mL) was added 1-ethyl-3-(3-dimethylaminopropyl)carbodiimide hydrochloride (0.040 g, 0.21 mmol). After 10 minutes, the mixture was allowed to warm to room temperature, and stirring was continued at room temperature overnight. The reaction mixture was partitioned between ethyl acetate and water. The separated organic phase was washed with saturated sodium bicarbonate aqueous solution, water and brine successively, dried over sodium sulfate, filtered and concentrated under reduced pressure to give N-{6-[4-(benzyloxy)phenyl] pyrimidin-4-yl}-N-{[tert-butyl(dimethyl)silyl]oxy}phenylalaninamide (0.075 g, 98%), which was used for the next step without further purification.

### N-{6-[4-(Benzyloxy)phenyl]pyrimidin-4-yl}-N-hydroxyphenylalaninamide

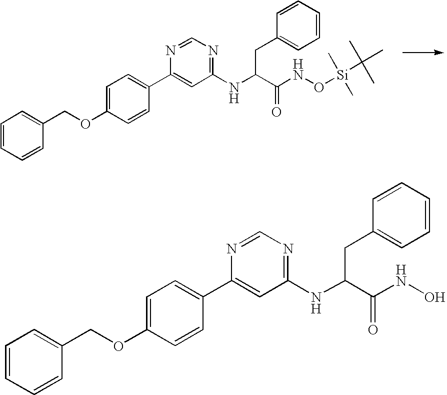

To a solution of N-{6-[4-(benzyloxy)phenyl]pyrimidin-4-yl}-N-{[tert-butyl(dimethyl)silyl]oxy}phenylalaninamide (0.050 g, 0.090 mmol) in THF (3 mL) was added a 1M solution of tetrabutylammonium fluoride in THF (1.0 mL, 1.0 mmol). After being stirred at room temperature for 1 hour, the reaction mixture was partitioned between ethyl acetate and water. The separated organic phase was washed with water and brine, dried over sodium sulfate, filtered, and concentrated under reduced pressure. The residue was purified by recrystallization from a mixture of methanol and water to give N-{6-[4-(benzyloxy)phenyl]pyrimidin-4-yl}-N-hydroxyphenylalaninamide (0.020 g, 50%) as a orange solid.

Melting point: 235-239° C.

Molecular weight: 440.50

Mass spectrometry: 441 (M+H)⁺

In vitro activity grade: A

¹H-NMR (500 MHz, CDCl₃): δ 2.92 (1H, br), 3.02 (1H, dd, J=5.3, 13.8 Hz), 4.75 (1H, br), 5.17 (2H, s), 6.95 (1H, br), 7.11 (2H, d, J=8.8 Hz), 7.17 (1, dd, J=6.9, 7.3 Hz), 7.23-7.30 (4H, m), 7.34 (1H, dd, J=6.9, 7.6 Hz), 7.40 (2H, dd, J=6.9, 7.6 Hz), 7.46 (2H, d, J=7.3 Hz), 7.62 (1H, br), 7.91 (2H, d, J=7.6 Hz), 8.41 (1H, s), 8.87 (1H, s), 10.76 (1H, s).

### Example 27-1

### N-[(Benzyloxy)carbonyl]phenylalaninamide

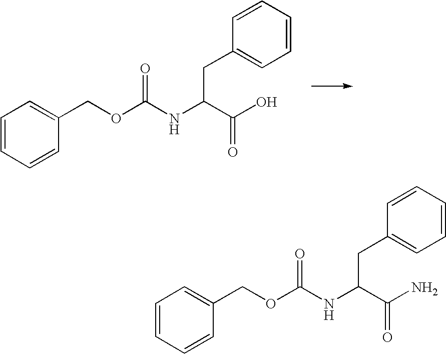

To a mixture of N-[(benzyloxy)carbonyl]phenylalanine (5.00 g, 16.70 mmol), di-tert-butyl carbonate (3.64 g, 20.88 mmol), ammonium hydrogen carbonate (1.58 g, 20.05 mmol) and 1,4-dioxane (25 mL) was added pyridine (0.800 mL, 9.89 mmol), and the mixture was stirred at room temperature overnight. Water (10 mL) was added to the mixture, which was stirred at room temperature for 30 minutes. The mixture was filtered, washed with water, and dried under reduced pressure to give N-[(benzyloxy)carbonyl]phenylalaninamide (3.97 g, 80%) as a white solid.

### Benzyl (1-cyano-2-phenylethyl)carbamate

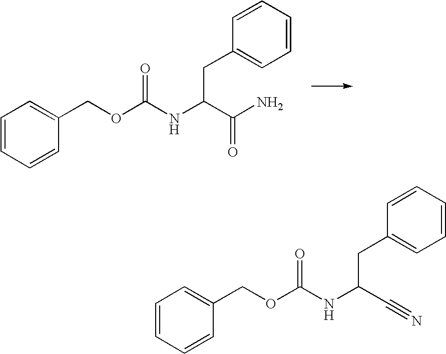

To a mixture of N-[(benzyloxy)carbonyl]phenylalaninamide (3.00 g, 10.06 mmol) and DMF (20 mL) was added cyanuric chloride (0.93 g, 5.03 mmol). The mixture was stirred at room temperature overnight. Water (10 mL) was added to the mixture, which was stirred at room temperature for 1 hour. The mixture was filtered, washed with diluted aqeous sodium hydrogen carbonate and water, and dried under reduced pressure to give benzyl (1-cyano-2-phenylethyl)carbamate (2.75 g, 98%) as a white solid.

### Benzyl [2-phenyl-1-(1H-tetrazol-5-yl)ethyl]carbamate

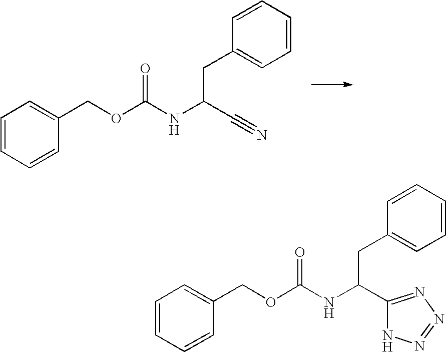

A mixture of benzyl (1-cyano-2-phenylethyl)carbamate (0.476 g, 1.70 mmol), sodium azide (0.221 g, 3.40 mmol), zinc dibromide (0.191 g, 0.85 mmol), water (7 mL) and 2-propanol (5 mL) was stirred at reflux for 6 hours. The mixture was added 1M aqueous hydrochloric acid (3 mL) and ethyl acetate (3 mL). The mixture was stirred at room temperature until no precipitate was formed. The mixture was partitionated between ethyl acetate and water. The separated organic phase was washed with brine, dried over sodium sulfate, filtered and concentrated under reduced pressure to give benzyl [2-phenyl-1-(1H-tetrazol-5-yl)ethyl]carbamate (0.495 g, 90%) as a colorless oil.

### Benzyl [2-phenyl-1-(2-{[2-(trimethylsilyl)ethoxy]methyl}-tetrazol-5-yl)ethyl]-carbamate

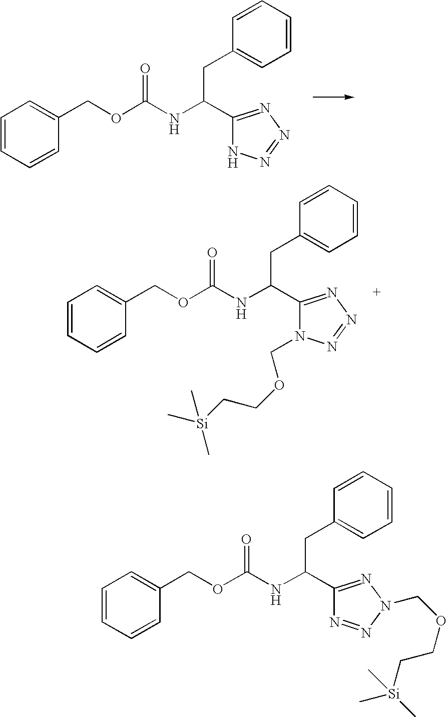

To a mixture of benzyl [2-phenyl-1-(1H-tetrazol-5-yl)ethyl]carbamate (0.495 g, 1.53 mmol) and DMF (10 mL) was added 2-(trimethylsilyl)ethoxymethyl chloride (0.281 mL, 1.68 mmol) and N,N-diisopropylethylamine (0.400 mL, 2.30 mmol) successively, and the mixture was stirred at room temperature for 1.5 hours. The mixture was partitionated between ethyl acetate and water. The separated organic phase was washed with brine, dried over sodium sulfate, filtered, and concentrated under reduced pressure. The residue was purified by preparative MPLC (hexane:ethyl acetate, 5:1) to give a mixture of benzyl [2-phenyl-1-(1-{[2-(trimethylsilyl)ethoxy]methyl}-1H-tetrazol-5-yl)ethyl]carbamate and benzyl [2-phenyl-1-(2-{[2-(trimethylsilyl)ethoxy]methyl}-2H-tetrazol-5-yl)ethyl]carbamate (0.534 g, 77%) as a colorless oil.

### [2-Phenyl-1-(1-{[2-(trimethylsilyl)ethoxy]methyl}-tetrazol-5-yl)ethyl]amine

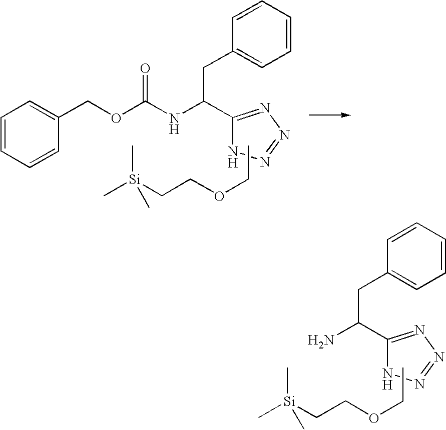

A mixture of Benzyl [2-phenyl-1-(2-{[2-(trimethylsilyl)ethoxy]methyl}-tetrazol-5-yl)ethyl]carbamate (0.534 g, 1.18 mmol), 10% palladium on activated carbon (0.060 g) and ethanol (10 mL) under a hydrogen atmosphere was stirred at room temperature for 12 hours. The resulting mixture was filtered through a Celite pad, and the filtrate was concentrated under reduced pressure. The residue was purified by column chromatography on silica-gel (chloroform: ethanol, 40:1) to give [2-phenyl-1-(1-{[2-(trimethylsilyl)ethoxy]methyl}tetrazol-5-yl)ethyl]amine (0.308 g, 82%) as a colorless oil.

### 4,6-Diiodopyrimidine

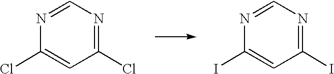

A mixture of 4,6-dichloropyrimidine (29.80 g, 200 mmol)and 48% aqueous hydrogen iodide (400 mL) was stirred at room temperature for 3 days in the dark. The mixture was filtered. The filter cake was added to a mixture of chloroform, 15% aqueous potasium carbonate (400 mL), and 10% aqueous sodium thiosulfate (400 mL). The mixtute was extracted with chloroform. The separated organic phase was dried over magnesium sulfate, filtered, and concentrated under reduced pressure. The residue was triturated with hexane to give 4,6-diiodopyrimidine (60.0 g, 90%) as a white solid.

### 6-Iodo-N-[2-phenyl-1-(1-{[2-(trimethylsilyl)ethoxy]methyl}-tetrazol-5-yl)ethyl]-pyrimidin-4-amine

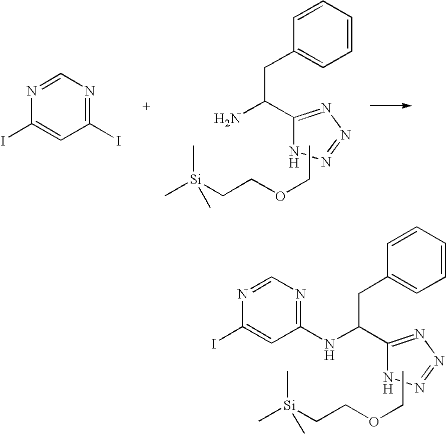

To a mixture of 4,6-diiodopyrimidine (0.104 g, 0.31 mmol), [2-phenyl-1-(1-{[2-(trimethylsilyl)ethoxy]methyl}-tetrazol-5-yl)ethyl]amine (0.100 g, 0.31 mmol), and ethanol (3 mL) was added N,N-diisopropylethylamine (0.060 mL, 0.34 mmol), and the mixture was stirred at reflux for 18 hours. The mixture was partitionated between ethyl acetate and water. The separated organic phase was washed with brine, dried over sodium sulfate, filtered, and concentrated under reduced pressure. The residue was purified by preparative TLC (chloform: ethanol, 40:1) to give 6-iodo-N-[2-phenyl-1-(1-{[2-(trimethylsilyl)ethoxy]methyl}-tetrazol-5-yl)ethyl]pyrimidin-4-amine (0.071 g, 43%)as a beige amorphous.

### 6-[4-(Benzyloxy)phenyl]-N-[2-phenyl-1-(1-{[2-(trimethylsilyl)ethoxy]methyl}-tetrazol-5-yl)ethyl]pyrimidin-4-amine

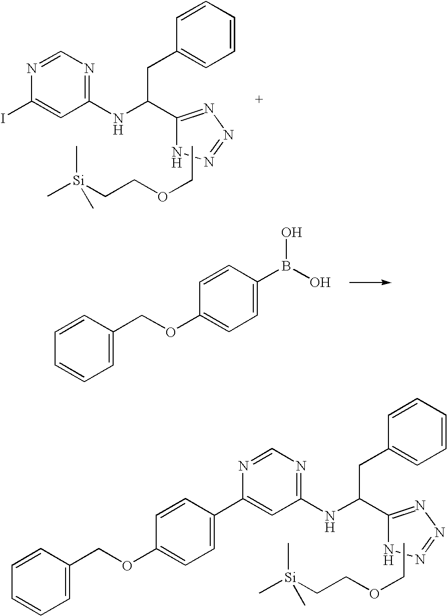

To a mixture of 6-iodo-N-[2-phenyl-1-(1-{2-(trimethylsilyl)ethoxy]methyl}-tetrazol-5-yl)ethyl]pyrimidin-4-amine (0.071 g, 0.14 mmol), 4-(benzyloxy)phenylboronic acid (0.031 g, 0.14 mmol) and DMF (2 mL) under an argon atmosphere was added 2N sodium carbonate aqueous solution (0.2 mL, 0.40 mmol) followed by tetrakis(triphenylphosphine)palladium (0.016 g, 0.01 mmol). The mixture was stirred at 80° C. overnight. After cooled to room temperature, the mixture was partitioned between ethyl acetate and water. The separated organic phase was washed with brine, dried over sodium sulfate, filtered and concentrated under reduced pressure. The crude product was purified by preparative TLC (chloroform: ethanol, 60:1) to give 6-[4-(benzyloxy)phenyl]-N-[2-phenyl-1-(1-{[2-(trimethylsilyl)ethoxy]methyl}-tetrazol-5-yl)ethyl]pyrimidin-4-amine (0.049 g, 63%) as a colorless oil.

### 6-[4-(Benzyloxy)phenyl]-N-[2-phenyl-1-(1H-tetrazol-5-yl)ethyl)pyrimidin-4-amine

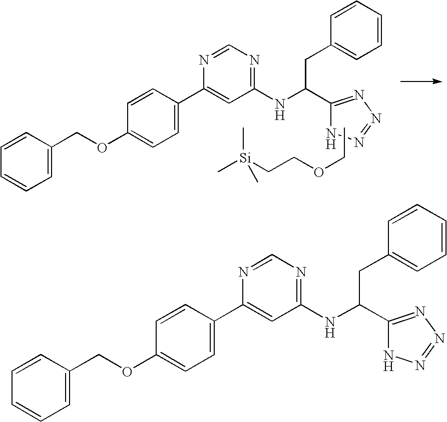

To a mixture of 6-[4-(benzyloxy)phenyl]-N-[2-phenyl-1-(1-{[2-(trimethylsilyl)ethoxy]methyl}-tetrazol-5-yl)ethyl]pyrimidin-4-amine (0.0273 g, 0.047 mmol) and 1,4-dioxane (1 mL) was added 1M aqueous hydrochloric acid (0.047 mL, 0.047 mmol), and the mixture was stirred at 60° C. overnight. The mixture was partitioned between ethyl acetate and water. The separated organic phase was washed with brine, dried over sodium sulfate, filtered and concentrated under reduced pressure. The residue was triturated with diethylether to give 6-[4-(benzyloxy)phenyl]-N-[2-phenyl-1-(1H-tetrazol-5-yl) ethyl]pyrimidin-4-amine (0.0089 g, 42%) as an off-white solid.

Melting point: 150° C.

Molecular weight: 449.52

Mass spectrometry: 450 (M+H)⁺

In vitro activity grade: A

¹H-NMR (500 MHz, MeOH-d4): δ 1.17(1H, m), 3.37 (1H, m), 3.49 (1H, m), 5.17 (2H, S), 5.93 (1H, br), 6.90 (1H, s), 7.14 (2H, d, J=8.5 Hz), 7.18 (1H, m), 7.23 (4H, m), 7.31 (1H, m), 7.37 (2H, m), 7.44 (2H, d, J=7.3 Hz), 7.55 (1H, m), 7.64 (1H, m), 7.78 (2H, d, J=8.8 Hz), 8.47 (1H, s).

### Example 27-2

In the similar manners as described in Example 27-1 above, compound in Example 27-2 as shown in Table 27 was synthesized.

TABLE EXAMPLE 27

| Ex. | | | MASS | | |
|---|---|---|---|---|---|
| No. | Structure | M.W. | (M + 1) | MP | In vitro |
| 27-2 | 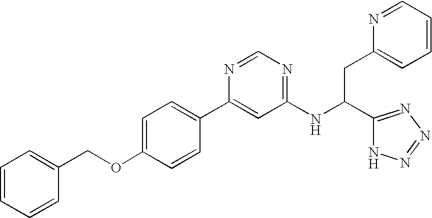 | 450.51 | 451 | 220z | B |

### Example 28-1

### tert-Butyl (2-chloropyridin-4-yl)carbamate

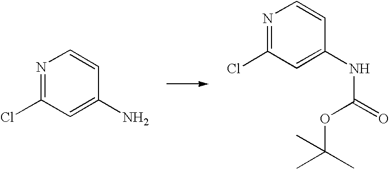

A mixture of 4-amino-2-chloropyridine (193 mg, 1.50 mmol), di-tert-butyl-dicarbonate (393 mg, 1.80 mmol) and 4-dimethylaminopyridine (1.8 mg, 0.02 mmol) in acetonitrile (5 mL) was stirred at room temperature for 18 hours. This mixture was concentrated under reduced pressure. The residue was purified by column chromatography on silica-gel (hexane/ethyl acetate, 20:1) to give tert-butyl (2-chloropyridin-4-yl)carbamate (250 mg, 73%).

### Ethyl N-(tert-butoxycarbonyl)-N-(2-chloropyridin-4-yl)glycinate

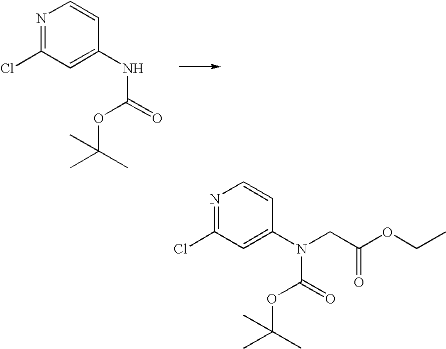

A mixture of tert-butyl (2-chloropyridin-4-yl)carbamate (250 mg, 1.09 mmol), ethyl bromoacetate (0.36 mL, 3.28 mmol) and potassium carbonate (755 mg, 5.47 mmol) in N,N-dimethylformamide (5 mL) was stirred at room temperature for 17 hours. The reaction mixture was concentrated under reduced pressure. The residue was purified by column chromatography on silica gel (hexane/ethyl acetate, 8:1) to give ethyl N(tert-butoxycarbonyl)-N-(2-chloropyridin-4-yl)glycinate (316 mg, 92%).

### Ethyl N-{2-[4-(benzyloxy)phenyl]pyridin-4-yl}-N-(tert-butoxycarbonyl)glycinate

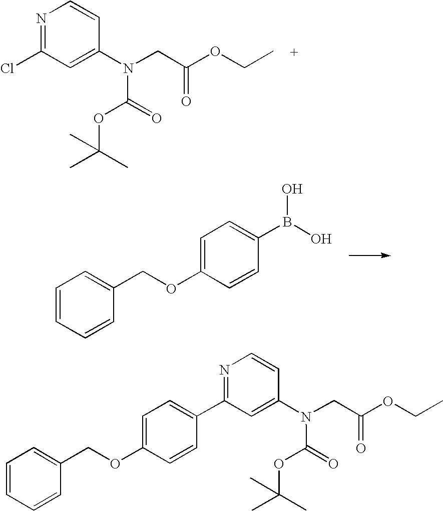

To a mixture of ethyl N-(tert-butoxycarbonyl)-N-(2-chloropyridin-4-yl)glycinate (316 mg, 1.00 mmol), tetrakis(triphenylphosphine)paladium (O) (58 mg, 0.05 mmol), potassium carbonate (416 mg, 3.01 mmol) and toluene (5 mL) was added portionwise (4-benzyloxyphenyl)boronic acid (343 mg, 1.51 mmol). The mixture was stirred at 100° C. for 19 hours. After cooled to room temperature, the reaction mixture was diluted with chloroform and filtered through a Celite pad. The filtrate was concentrated under reduced pressure. The residue was purified by preparative TLC (hexane/ethyl acetate, 1:1) to give ethyl N-{2-[4-(benzyloxy)phenyl]pyridin-4-yl}-N-(tert-butoxycarbonyl)glycinate (334 mg, 72%).

### Ethyl N-{2-[4-(benzyloxy)phenyl]pyridin-4-yl}-N-(tert-butoxycarbonyl)phenylalaninate

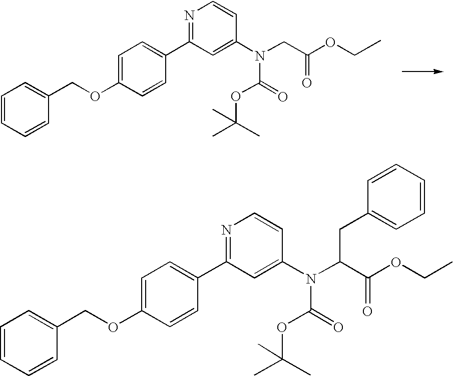

To a solution of ethyl N{2-[benzyloxy)phenyl]pyridin-4-yl}-N-(tert-butoxycarbonyl)glycinate (314 mg, 0.68 mmol) and benzyl bromide (0.12 mL, 1.02 mmol) in hexamethylphosphoric triamide (0.7 mL) and tetrahydrofuran (7 mL) at −78° C. was added dropwise a 1M solution of sodium bis(trimethylsilyl)amide (1.03 mL, 1.03 mmol) in tetrahydrofuran. This mixture was stirred for 3 hours and warmed to −10° C., and then quenched with saturated ammonium chloride solution and extracted with ethyl acetate. The separated organic phase was washed with water and brine, dried over magnesium sulfate, and concentrated under reduced pressure. The residue was purified by column chromatography on silica-gel (hexane/ethyl acetate, 5:1) to give ethyl N-{2-[4-(benzyloxy)phenyl]pyridin-4-yl}-N-(tert-butoxycarbonyl)phenylalaninate (50 mg, 13%).

### Ethyl N {2-[4-(benzyloxy)phenyl]pyridin-4-yl}phenylalaninate

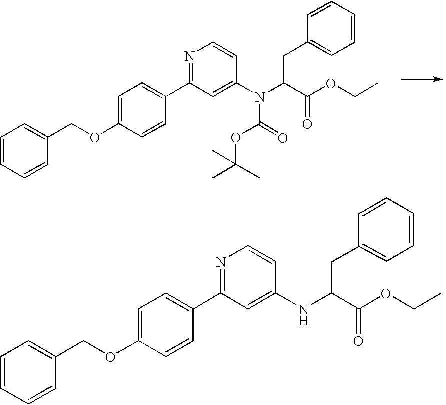

A solution of ethyl N-{2-[4-(benzyloxy)phenyl]pyridin-4-yl}-N-(tert-butoxycarbonyl)phenylalaninate (50 mg, 0.09 mmol) in dioxane (0.5 mL) was added dropwise 4N hydrogen chloride in dioxane (0.5 mL). This mixture was stirred for 5 hours at room temperature. The reaction mixture was concentrated under reduced pressure. The residue was purified by preparative TLC (hexane/ethyl acetate, 1:1) to give ethyl N{2-[4-(benzyloxy)phenyl]pyridin-4-yl}phenylalaninate (21 mg, 51%).

### N-{2-[4-(Benzyloxy)phenyl]pyridin-4-yl}phenylalanine

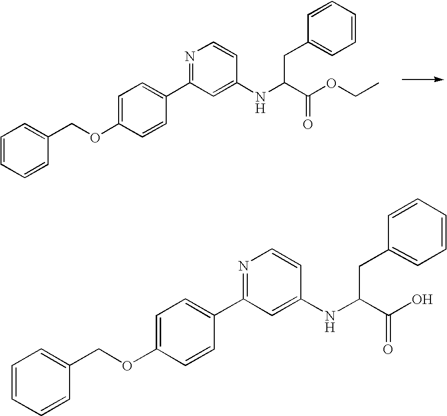

A mixture of ethyl N-{2-[4-(benzyloxy)phenyl]pyridin-4-yl}phenylalaninate (21 mg, 0.05 mmol) in methanol (0.3 mL) and tetrahydrofuran (0.3 mL) was added dropwise 1N aqueous sodium hydroxide solution (0.3 mL, 0.3 mmol). The mixture was stirred at room temperature for 2 hours, then acidified with 1N hydrochloric acid, and concentrated under reduced pressure. The residual precipitate was collected by filtration, washed with water, and dried under reduced pressure to give N-{2-[4-(benzyloxy)phenyl]pyridin-4-yl}phenylalanine (14 mg, 71%) as a white solid.

Melting point: 137-139° C.

Molecular weight: 424.5

Mass spectrometry: 425 (M+H)⁺

In vitro activity grade: A

¹H-NMR (500 MHz, DMSO-d6): δ 3.00 (1H, dd, J=8.8, 14.0 Hz), 3.18 (1H, dd, J=5.0, 14.0 Hz), 3.76 (3H, s), 4.59 (1H, s), 5.18 (2H, s), 6.60 (1H, s), 7.02 (1H, s), 7.12 (2H, d, J=8.5 Hz), 7.19 (1H, t, J=7.0 Hz), 7.20-7.36 (6H, m), 7.41 (2H, t, J=7.2 Hz), 7.47 (2H, d, J=7.0 Hz), 7.83 (2H, d, J=8.8 Hz), 8.06 (1H, d, J=6.3 Hz), 13.1 (1H, br.s).

### Examples 28-2 to 28-4

In the similar manners as described in Example 28-1 above, compounds in Examples 28-2 to 284 as shown in Table 28 were synthesized.

TABLE EXAMPLE 28

| Ex. | | | MASS | | |
|---|---|---|---|---|---|
| No. | Structure | M.W. | (M + 1) | MP | In vitro |
| 28-2 | 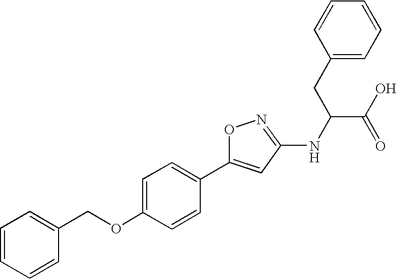 | 414.47 | 415 | 195Z | A |
| 28-3 | 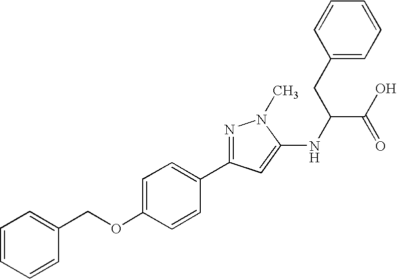 | 427.51 | 428 | 171 | C |
| 28-4 | 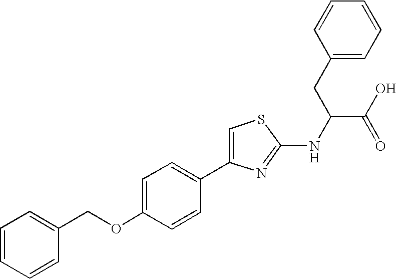 | 430.53 | 431 | amorphous | C |

### Example 29-1

### 1-[4-(Benzyloxy)phenyl]ethanone

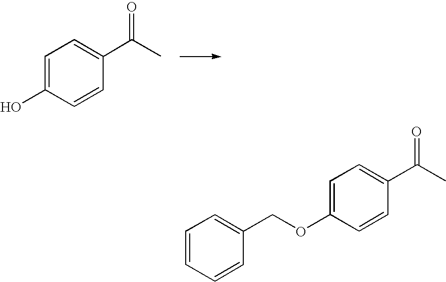

To a solution of 1-(4-hydroxyphenyl)ethanone (2.0 g, 14.69 mmol) and benzylchloride (2.23 g, 17.63 mmol) in DMF (40 mL) were added potassium carbonate (2.64 g, 19.10 mmol) and sodium iodide (0.22 g, 1.47 mmol), and the mixture was stirred at room temperature overnight. The mixture was concentrated under reduced pressure, and the residue was partitioned between ethyl acetate and water. The separated organic phase was dried over sodium sulfate, filtered and concentrated under reduced pressure. The residual solid was triturated with diisopropylether to give 1-[4-(benzyloxy)phenyl]ethanone (2.81 g, 85%) as yellowish granules.

### (2E)-1-[4-(Benzyloxy)phenyl]-3-(dimethylamino)-2-propen-1-one

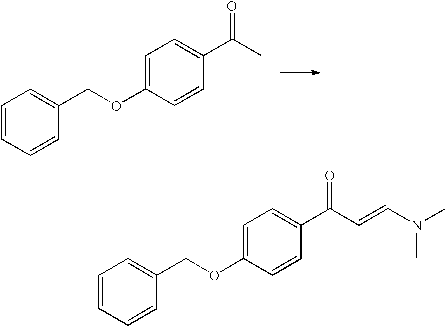

A mixture of 1-[4-(benzyloxy)phenyl]ethanone (2.0 g, 8.84 mmol) and N-[tert-butoxy(dimethylamino)methyl]-N,N-dimethylamine (2.31 g, 13.26 mmol) in toluene (12 mL) was stirred under reflux for 3 hours. The volatiles were removed by evaporation and the residual solid was triturated with diisopropylether to give (2E)-1-[4-(benzyloxy)phenyl]-3-(dimethylamino)-2-propen-1-one (2.51 g, quantitative) as a yellow powder.

### 4-[4-(Benzyloxy)phenyl]-2-(methylsulfanyl)pyrimidine

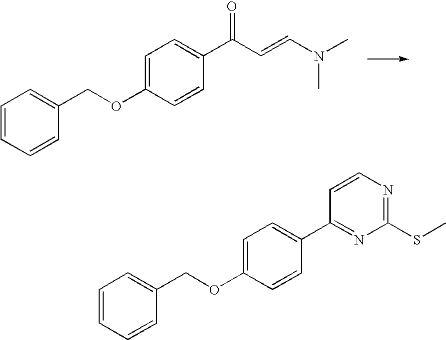

To a solution of (2E)-1-[4-(benzyloxy)phenyl]-3-(dimethylamino)-2-propen-1-one (2.51 g, 9.39 mmol) and thiourea (1.43 g, 18.78 mmol) in ethanol (25 mL) was added portionwise sodium ethoxide (1.49 g, 21.87 mmol), and the mixture was stirred at 70° C. for 2 hours. After the mixture being cooled, iodomethane (6.62 g, 46.94 mmol) was added, and the stirring was continued overnight. The mixture was filtered to remove the precipitate, which was rinsed with ethyl acetate. The combined filtrates were concentrated under reduced pressure. The residue was partitioned between ethyl acetate and water. The separated organic phase was washed with brine, dried over sodium sulfate, filtered, and concentrated under reduced pressure. The residue was purified by column chromatography on silica-gel (n-hexane:ethyl acetate, 7:1-3:1) to give 4-[4-(benzyloxy)phenyl]-2-(methylsulfanyl)pyrimidine (2.47 g, 85%) as a slightly yellow solid.

### 4-[4-(Benzyloxy)phenyl]-2-(methylsulfonyl)pyrimidine

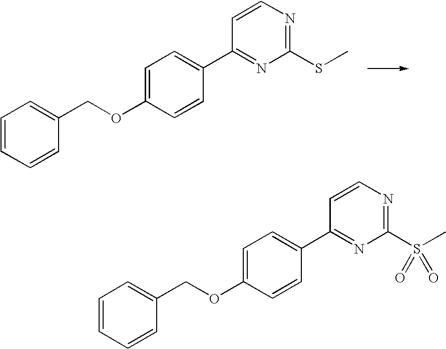

To a cold (0° C.) solution of 4-[4-(benzyloxy)phenyl]-2-(methylsulfanyl)pyrimidine (0.50 g, 1.62 mmol) in dichloromethane (6.0 mL) was added m-chloroperbenzoic acid (75%, 0.75 g, 3.24 mmol), and the mixture was stirred for 4 hours. The mixture was poured into a mixture of 5% aqueous sodium thiosulfate and dichloromethane. The organic phase was separated, washed with saturated aqueous sodium bicarbonate, dried over sodium sulfate, filtered, and concentrated under reduced pressure to give a crude 4-[4-(benzyloxy)phenyl]-2-(methylsulfonyl)pyrimidine (0.54 g, 98%) as a yellowish solid, which was used for the next step without further purification.

### tert-Butyl N-{4-[4-(benzyloxy)phenyl] pyrimidin-2-yl}phenylalaninate

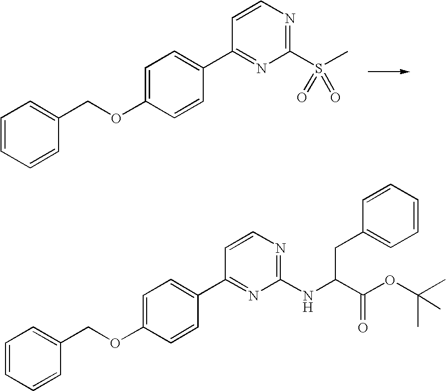

A mixture of 4-[4-(benzyloxy)phenyl]-2-(methylsulfonyl)pyrimidine (300 mg, 0.88 mmol) and D,L-phenylalanine tert-butyl ester (585 mg, 2.64 mmol) was stirred at 120° C. overnight After being cooled to room temperature, the mixture was purified by column chromatography on silica-gel (chloroform) to give tert-butyl N-{4-[4-(benzyloxy)phenyl] pyrimidin-2-yl}-phenylalaninate (260 mg, 61%) as a yellowish solid.

### N-{4-[4-(Benzyloxy)phenyl] pyrimidin-2-yl}phenylalanine

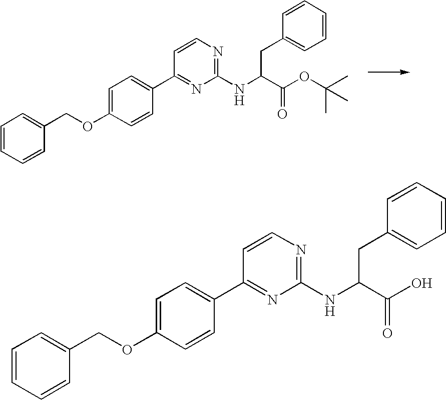

To a solution of tert-butyl N-{4-[4-(benzyloxy)phenyl] pyrimidin-2-yl}-phenylalaninate (0.26 g, 0.54 mmol) in tetrahydrofuran (2.5 mL) and ethanol (2.5 mL) was added dropwise 1N LiOH aqueous solution (0.82 mL, 0.82 mmol), and the mixture was stirred under reflux overnight. After cooled to room temperature, the mixture was concentrated under reduced pressure. The residue was suspended in water and neutralized with 1N HCl solution (0.82 mL). The resultant precipitate was collected by filtration and washed successively with water and ethyl acetate to give N-{4-[4(benzyloxy)phenyl] pyrimidin-2-yl}phenylalanine (0.117 g, 51%) as a colorless powder.

Melting point: 174° C.

Molecular weight: 425.49

Mass spectrometry: 426 (M+H)⁺

In vitro activity grade: A

¹H-NMR (500 MHz, CD₃OD): δ 3.09 (1H, dd, J=13.6, 7.3 Hz), 4.55 (1H, bs), 5.16 (2H, s), 6.98 (1H, d, J=5.4 Hz), 7.07 (2H, dd, J=6.9, 2.2 Hz), 7.09 (1H, t, J=7.6 Hz), 7.17 (1H, t, J=7.6 Hz), 7.24 (1H, d, J=7.9 Hz), 7.31 (1H, t, J=7.3 Hz), 7.38 (1H, t, J=7.3 Hz), 7.46. (1H, d, J=7.6 Hz), 8.05 (2H, bs), 8.14 (1H, bs).

1. A phenyl or heteroaryl amino alkane derivative of the formula (I), its tautomeric or stereoisomeric form, or a salt thereof: 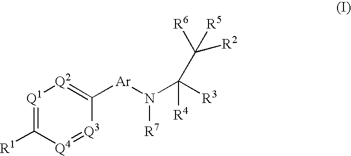
wherein
Ar represents phenylene or a 5 or 6 membered heteroaryl containing 1-3 heteroatoms selected from the group consisting of O, N and S,
wherein
said phenylene or 5 or 6 membered heteroaryl optionally has one or more substituents selected from the group consisting of halogen, hydroxy, cyano, nitro, amino, N-(C₁₋₆)alkylamino, N,N-di(C₁₋₆)alkylamino, formyl, (C₁₋₆)alkylthio, (C₁₋₆)alkoxy and (C₁₋₆)alkyl optionally substituted by hydroxy, or mono-, di- or tri-halogen;
Q¹, Q², Q³ and Q⁴ independently represent CH, CR¹⁰ or N;
wherein
R¹⁰ represents halogen, cyano, amino, nitro, formyl, hydroxymethyl, methylthio, (C₁₋₆)alkyl optionally substituted by mono-, di- or tri-halogen, or (C₁₋₆)alkoxy optionally substituted by phenyl;
R¹ represents —OR¹¹, —CH₂NHR¹¹, —C(O)R¹¹, —C(O)NHR¹¹, —SR¹¹, —SOR¹¹, —SO₂R¹¹, —NHR¹¹, —NHC(O)OR¹¹, —NHC(O)NR¹¹, —NHC(O)R¹¹, —NHSO₂R¹¹, hydrogen, hydroxy, or halogen, or
a saturated or unsaturated 3-10 membered mono- or bi-cyclic ring optionally having one or two heteroatoms selected independently from O and N or
(C₁₋₆)alkyl optionally substituted by aryloxyimino, (C₁₋₆)alkoxy optionally substituted by aryl or heteroaryl, or
a saturated or unsaturated 3-10 membered mono- or bi-cyclic ring optionally having one or two heteroatoms selected independently from O and N, or
(C₂₋₆)alkenyl optionally substituted by a saturated or unsaturated 3-10 membered mono- or bi-cyclic ring optionally having one or two heteroatoms selected independently from O and N, or
(C₂₋₆)alkynyl optionally substituted by a saturated or unsaturated 3-10 membered mono- or bi-cyclic ring optionally having one or two heteroatoms selected independently from O and N,
in any of which the saturated or unsaturated 3-10 membered mono- or bi-cyclic ring may be optionally substituted by one or more substituents selected from the group consisting of
halogen, hydroxy, cyano, nitro, (C₁₋₆) alkylthio,
(C₁₋₆)alkyl optionally substituted by mono-, di-, or tri-halogen,
(C₁₋₆)alkoxy optionally substituted by mono-, di-, or tri-halogen,
aryl optionally substituted by nitro, (C₁₋₆)alkyl or (C₁₋₆)alkoxy,
aralkyl optionally, at the aryl moiety, substituted by nitro, (C₁₋₆)alkyl or (C₁₋₆)alkoxy,
and
aryloxy optionally substituted by nitro, (C₁₋₆)alkyl or (C₁₋₆)alkoxy,
wherein
R¹¹ represents (C₁₋₆)alkoxy(C₁₋₆)alkylene,
a saturated or unsaturated 3-10 membered mono- or bi-cyclic ring optionally having one or two heteroatoms selected independently from O and N,
(C₁₋₆)alkyl optionally substituted by mono-, di- or tri-halogen or a saturated or unsaturated 3-10 membered mono- or bi-cyclic ring optionally having one or two heteroatoms selected independently from O and N,
(C₂₋₆)alkenyl optionally substituted by a saturated or unsaturated 3-10 membered mono- or bi-cyclic ring optionally having one or two heteroatoms selected independently from O and N, or
(C₂₋₆)alkynyl optionally substituted by a saturated or unsaturated 3-10 membered mono- or bi-cyclic ring optionally having one or two heteroatoms selected independently from O and N,
in any of which the saturated or unsaturated 3-10 membered mono- or bi-cyclic ring may be optionally substituted by one or more substituents selected from the group consisting of
halogen, hydroxy, cyano, nitro,
(C₁₋₆)alkoxy optionally substituted by mono-, di-, or tri-halogen, and
(C₁₋₆)alkyl optionally substituted by mono-, di-, or tri-halogen;
R² represents hydrogen, hydroxy, amino, N-(C₁₋₆)alkylamino, (C₂₋₆)alkenyl, (C₂₋₆)alkynyl, (C₃₋₇)cycloalkyl, (C₁₋₆)alkylthio, (C₁₋₆)alkylsulfonyl, aryl, heteroaryl, or
(C₁₋₆)alkyl optionally substituted by mono-, di- or tri-halogen, (C₁₋₆)alkylsulfonyl, (C₁₋₆)alkylthio, aryl or heteroaryl, or
(C₁₋₆)alkoxy optionally substituted by mono-, di- or tri-halogen, (C₁₋₆)alkylsulfonyl, aryl or heteroaryl,
in any of which the aryl or heteroaryl may optionally be substituted by one or more substituents selected from the group consisting of halogen, hydroxy, nitro, amino, N-(C₁₋₆)alkylamino, N,N-di(C₁₋₆)alkylamino, N-(4,5-dihydro-1H-imidazole)amino, (C₁₋₆)alkyl, phenyl, a 5 or 6 membered heteroaryl containing 1 to 3 heteroatoms selected from the group of O, N, and S,
and
(C₁₋₆)alkoxy optionally substituted by morpholino, amino, N-(C₁₋₆)alkylamino, or N,N-di(C₁₋₆) alkylamino;
R³ represents hydrogen or (C₁₋₆) alkyl optionally substituted mono-, di- or tri-halogen;
R⁴ represents carboxy, tetrazolyl or N-(hydroxy)aminocarbonyl;
R⁵ represents hydrogen, (C₁₋₆)alkoxy, aryl, heteroaryl or (C₁₋₆)alkyl optionally substituted by mono-, di- or tri-halogen;
R⁶ represents hydrogen or (C₁₋₆)alkyl optionally substituted by mono-, di- or tri-halogen; and
R⁷ represents hydrogen, or (C₁₋₆)alkyl.

2. The phenyl or heteroaryl amino alkane derivative of the formula (I), its tautomeric or stereoisomeric form, or a salt thereof as claimed in claim 1,
wherein
Ar represents 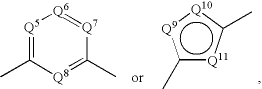
Q⁵, Q⁶, Q⁷ and Q⁸ independently represent CH, CR⁸ or N,
Q⁹, Q¹⁰ and Q¹² independently represent O, S, CH, CR⁸, CH₂, NH, or NR⁹,
wherein
R⁸ represents halogen, cyano, amino, nitro, formyl, hydroxymethyl, methylthio, (C₁₋₆)alkoxy, or (C₁₋₆)alkyl optionally substituted by mono-, di- or tri-halogen,
R⁹ represents (C₁₋₆)alkyl;
Q¹, Q², Q³ and Q⁴ independently represent CH, CR¹⁰ or N,
wherein
R¹⁰ represents halogen, amino, nitro, formyl, hydroxymethyl, methylthio, (C₁₋₆)alkyl optionally substituted by mono-, di- or tri-halogen, or (C₁₋₆)alkoxy optionally substituted by phenyl;
R¹ represents —OR¹¹, —CH₂NHR¹¹, —C(O)R¹¹, —C(O)NHR¹¹, —SR¹¹, —SOR¹¹, —SO₂R¹¹, —NHR¹¹, —NHC(O)R¹¹, —NHC(O)OR¹¹, —NHC(O)NR¹¹, —NHSO₂R¹¹, hydrogen, hydroxy, halogen, or
a saturated or unsaturated 3-10 membered mono- or bi-cyclic ring optionally having one or two heteroatoms selected independently from O and N, or
(C₁₋₆)alkyl optionally substituted by aryloxyimino, (C₁₋₆)alkoxy optionally substituted by aryl or hereoaryl, or a saturated or unsaturated 3-10 membered mono- or bi-cyclic ring optionally having one or two heteroatoms selected independently from O and N, or
(C₂₋₆)alkenyl optionally substituted by a saturated or unsaturated 3-10 membered mono- or bi-cyclic ring optionally having one or two heteroatoms selected independently from O and N, or
(C₂₋₆)alkynyl optionally substituted by a saturated or unsaturated 3-10 membered mono- or bi-cyclic ring optionally having one or two heteroatoms selected independently from O and N,
in any of which the saturated or unsaturated 3-10 membered mono- or bi-cyclic ring may be optionally substituted by one or more substituents selected from the group consisting of
halogen, hydroxy, cyano, nitro, (C₁₋₆)alkylthio,
(C₁₋₆)alkyl optionally substituted by mono-, di-, or tri-halogen,
(C₁₋₆)alkoxy optionally substituted by mono-, di-, or tri-halogen,
aryl optionally substituted by nitro, (C₁₋₆)alkyl or (C₁₋₆) alkoxy,
aralkyl optionally, at the aryl moiety, substituted by nitro, (C₁₋₆)alkyl or (C₁₋₆)alkoxy,
and
aryloxy optionally substituted by nitro, (C₁₋₆)alkyl or (C₁₋₆)alkoxy,
wherein
R¹¹ represents (C₁₋₆)alkoxy(C₁₋₆)alkylene,
a saturated or unsaturated 3-10 membered mono- or bi-cyclic ring optionally having one or two heteroatoms selected independently from O and N,
(C₁₋₆)alkyl optionally substituted by mono-, di- or tri-halogen or a saturated or unsaturated 3-10 membered mono- or bi-cyclic ring optionally having one or two heteroatoms selected independently from O and N,
(C₂₋₆)alkenyl optionally substituted by a saturated or unsaturated 3-10 membered mono- or bi-cyclic ring optionally having one or two heteroatoms selected independently from O and N, or
(C₂₋₆)alkynyl optionally substituted by a saturated or unsaturated 3-10 membered mono- or bi-cyclic ring optionally having one or two heteroatoms selected independently from O and N,
in any of which the saturated or unsaturated 3-10 membered mono- or bi-cyclic ring may be optionally substituted by one or more substituents selected from the group consisting of
halogen, hydroxy, cyano, nitro,
(C₁₋₆)alkoxy optionally substituted by mono-, di-, or tri-halogen, and
(C₁₋₆)alkyl optionally substituted by mono-, di-, or tri-halogen;
R² represents hydrogen, hydroxy, amino, N-(C₁₋₆)alkylamino, (C₂₋₆)alkenyl, (C₂₋₆)alkynyl, (C₃₋₇)cycloalkyl, (C₁₋₆)alkylthio, (C₁₋₆)alkylsulfonyl, aryl, heteroaryl,
(C₁₋₆)alkyl optionally substituted by mono-, di- or tri-halogen, (C₁₋₆)alkylsulfonyl, (C₁₋₆)alkylthio, aryl or heteroaryl, or
(C₁₋₆)alkoxy optionally substituted by mono-, di- or tri-halogen, (C₁₋₆)alkylsulfonyl, aryl or heteroaryl,
in any of which the aryl or heteroaryl may optionally be substituted by one or more substituents selected from the group consisting of halogen, hydroxy, nitro, amino, N-(C₁₋₆)alkylamino, N,N-di(C₁₋₆)alkylamino, N-(4,5-dihydro-1H-imidazole)amino, (C₁₋₆)alkyl, phenyl, a 5 or 6 membered heteroaryl containing 1 to 4 heteroatoms selected from the group of O, N, and S,
and
(C₁₋₆)alkoxy optionally substituted by morpholino, amino, N-(C₁₋₆)alkylamino, or N,N-di(C₁₋₆) alkylamino;
R³ represents hydrogen, or C₁₋₆ alkyl optionally substituted mono, di- or tri-halogen;
R⁴ represents carboxy, tetrazolyl or N-(hydroxy)aminocarbonyl;
R⁵ represents hydrogen, (C₁₋₆)alkyl, (C₁₋₆)alkoxy, aryl or heteroaryl;
R⁶ represents hydrogen; and
R⁷ represents hydrogen, or (C₁₋₆)alkyl.

3. The phenyl or heteroaryl amino alkane derivative of the formula (I), its tautomeric or stereoisomeric form, or a salt thereof as claimed in claim 1,
wherein
Ar represents 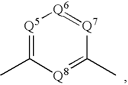
Q⁵, Q⁶, Q⁷ and Q⁸ independently represent CH, CR⁸ or N,
wherein
R⁸ represents halogen, cyano, amino, nitro, formyl, hydroxymethyl, methylthio, (C₁₋₆)alkoxy, or (C₁₋₆)alkyl optionally substituted by mono-, di-, or tri-halogen;
Q¹, Q², Q³ and Q⁴ independently represent CH, CR¹⁰ or N,
wherein
R¹⁰ represents halogen, amino, nitro, formyl, trifluoromethyl, hydroxymethyl, methylthio or benzyloxy;
R¹ represents —OR¹¹, —CH₂OR¹¹, —CH₂NHR¹¹, —C(O)R¹¹, —C(O)NHR¹¹, —SR¹¹, —SOR¹¹, —SO₂R¹¹, —NHR¹¹, —NHC(O)R¹¹, —NHC(O)OR¹¹, —NHC(O)NR¹¹, —NHSO₂R¹¹, hydrogen, hydroxy, halogen,
(C₁₋₆)alkyl optionally substituted by phenoxyimino, (C₁₋₆)alkoxy or R¹²,
wherein
said (C₁₋₆) alkoxy optionally substituted by pyrrolyl, pyrazolyl, imidazolyl, phenyl, pyridyl, pyrazinyl, pyridazinyl, pyrimidinyl, benzodioxolyl, naphthyl, indolyl, isoindolyl, quinolyl, isoquinolyl, or dihydroisoquinolyl,
(C₂₋₆)alkenyl optionally substituted by R¹²,
(C₂₋₆)alkynyl optionally substituted by R¹², or
one of the following carbocyclic or heterocyclic rings selected from the group consisting of cyclopropyl, cyclobutyl, cyclopentyl, cyclohexyl, pyrrolidinyl pyrrolyl, piperidino, piperidyl, piperazinyl, pyrazolyl, imidazolyl, phenyl, pyridyl, pyrazinyl, pyridazinyl, pyrimidinyl, benzodioxolyl, naphthyl, indolyl, isoindolyl, quinolyl, isoquinolyl, and dihydroisoquinolyl,
in any of which the carbocyclic or heterocyclic rings may optionally be substituted with 1 to 3 substituents selected from the group consisting of hydroxy, halogen, nitro, cyano, carboxy, amino, N-(C₁₋₆)alkylamino, N,N-di(C₁₋₆)alkylamino, (C₁₋₆)alkylthio, phenyl, phenoxy, benzyl, naphthyl, (C₁₋₆)alkyl optionally substituted by mono-,di- or tri-halogen, and (C₁₋₆)alkoxy optionally substituted by mono-, di- or tri-halogen, or phenyl;
wherein
R¹¹ represents (C₁₋₆)alkoxy (C₁₋₆)alkylene,
(C₁₋₆)alkyl optionally substituted by R¹⁰¹,
(C₂₋₆)alkenyl optionally substituted by R¹⁰¹,
(C₂₋₆)alkynyl optionally substituted by R¹⁰¹, or
one of the following carbocyclic or heterocyclic rings selected from the group consisting of cyclopropyl, cyclobutyl, cyclopentyl, cyclohexyl, pyrrolidinyl pyrrolyl, piperidino, piperidyl, piperazinyl, pyrazolyl, imidazolyl, phenyl, pyridyl, pyrazinyl, pyridazinyl, pyrimidinyl, benzodioxolyl, naphthyl, indolyl, isoindolyl, quinolyl, isoquinolyl, and dihydroisoquinolyl,
in any of which the carbocyclic or heterocyclic rings may optionally be substituted with 1 to 3 substituents selected from the group consisting of hydroxy, halogen, nitro, cyano, carboxy, amino, N-(C₁₋₆alkyl)amino, N,N-di(C₁₋₆alkyl)amino, (C₁₋₆)alkylthio, phenyl, phenoxy, benzyl, naphthyl, (C₁₋₆)alkyl optionally substituted by mono-, di- or tri-halogen, and (C₁₋₆)alkoxy optionally substituted by mono-, di- or tri-halogen,
R¹⁰¹ represents one of the following carbocyclic or heterocyclic rings selected from the group consisting of cyclopropyl, cyclobutyl, cyclopentyl, cyclohexyl, pyrrolidinyl, pyrrolyl, piperidino, piperidyl, piperazinyl, pyrazolyl, imidazolyl, phenyl, pyridyl, pyrazinyl, pyridazinyl, pyrimidinyl, benzodioxolyl, naphthyl, indolyl, isoindolyl, quinolyl, isoquinolyl, and dihydroisoquinolyl,
in any of which the carbocyclic or heterocyclic rings may optionally be substituted with 1 to 3 substituents selected from the group consisting of hydroxy, halogen, nitro, cyano, carboxy, amino, N-(C₁₋₆alkyl)amino, N,N-di(C₁₋₆alkyl)amino, (C₁₋₆)alkylthio, phenyl, phenoxy, benzyl, naphthyl, (C₁₋₆)alkyl optionally substituted by mono-, di- or tri-halogen, and (C₁₋₆)alkoxy optionally substituted by mono-, di-, or tri-halogen;
R¹² represents one of the following carbocyclic or heterocyclic rings selected from the group consisting of cyclopropyl, cyclobutyl, cyclopentyl, cyclohexyl, pyrrolidinyl pyrrolyl, piperidino, piperidyl, piperazinyl, pyrazolyl, imidazolyl, phenyl, pyridyl, pyrazinyl, pyridazinyl, pyrimidinyl, benzodioxolyl, naphthyl, indolyl, isoindolyl, quinolyl, isoquinolyl, and dihydroisoquinolyl,
in any of which the carbocyclic or heterocyclic rings may optionally be substituted with 1 to 3 substituents selected from the group consisting of hydroxy, halogen, nitro, cyano, carboxy, amino, N-(C₁₋₆alkyl)amino, N,N-di(C₁₋₆alkyl)amino, (C₁₋₆)alkylthio, phenyl, phenoxy, benzyl, naphthyl, (C₁₋₆)alkyl optionally substituted by mono-, di- or tri-halogen, and (C₁₋₆)alkoxy optionally substituted by mono-, di- or tri-halogen;
R² represents hydrogen, hydroxy, amino, N-(C₁₋₆)alkylamino, (C₂₋₆)alkenyl, (C₂₋₆)alkynyl, (C₃₋₇)cycloalkyl, pyrimidinyl, indolyl, pyridyl,
(C₁₋₆)alkoxy optionally substituted by amino, N-(C₁₋₆)alkylamino, N,N-di(C₁₋₆)alkylamino, or phenyl,
(C₁₋₆)alkyl optionally substituted by phenyl, mono-, di- or tri-halogen, (C₁₋₆)alkylthio, or (C₁₋₆)alkylsulfonyl,
phenyl optionally substituted by halogen, hydroxy, nitro, amino, N-(C₁₋₆)alkylamino, N-(dihydroimidazolyl)amino, (C₁₋₆)alkyl, or (C₁₋₆)alkoxy optionally substituted by R²¹,
wherein
R²¹ represents amino, N-(C₁₋₆)alkylamino, N,N-di(C₁₋₆)alkylamino, or morpholino;
R³ represents hydrogen, or (C₁₋₆)alkyl optionally substituted by mono-, di- or tri-halogen;
R⁴ represents carboxy, tetrazolyl or N-(hydroxy)aminocarbonyl;
R⁵ represents hydrogen, (C₁₋₆)alkyl, (C₁₋₆)alkoxy, phenyl, pyridyl, pyrazinyl, pyrimidinyl, or pyridazinyl;
R⁶ represents hydrogen; and
R⁷ represents hydrogen or (C₁₋₆)alkyl.

4. The phenyl or heteroaryl amino alkane derivative of the formula (I), its tautomeric or stereoisomeric form, or a salt thereof as claimed in claim 1,
wherein
Ar represents 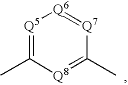
Q⁵ and Q⁷ independently represent CH or N,
Q⁶ and Q⁸ independently represent CH or CR⁸,
wherein
R⁸ represents halogen, cyano, amino, nitro, formyl, hydroxymethyl, methylthio or trifluoromethyl;
Q¹ independently represent represents CH or CR¹⁰,
wherein
R¹⁰ represents halogen, cyano, amino, nitro, formyl, trifluoromethyl, hydroxymethyl, methylthio or benzyloxy;
Q², Q³ and Q⁴ represent CH;
R¹ represents —OR¹¹, —CH₂NHR¹¹, —C(O)R¹¹, —C(O)NHR¹¹, —SR¹¹, —SOR¹¹, —SO₂R¹¹, —NHR¹¹, —NHC(O)R¹¹, —NHC(O)OR¹¹, —NHC(O)NR¹¹, —NHSO₂R¹¹, hydrogen, hydroxy, halogen,
(C₁₋₆)alkyl optionally substituted by (C₁₋₆)alkoxy or R¹²,
wherein
said (C₁₋₆) alkoxy optionally substituted by pyrrolyl, pyrazolyl, imidazolyl, phenyl, pyridyl, pyrazinyl, pyridazinyl, pyrimidinyl, benzodioxolyl, naphthyl, indolyl, isoindolyl, quinolyl, isoquinolyl, or dihydroisoquinolyl,
(C₂₋₆)alkenyl optionally substituted by R¹²,
(C₂₋₆)alkynyl optionally substituted by R¹², or
one of the following carbocyclic or heterocyclic rings selected from the group consisting of cyclopropyl, cyclobutyl, cyclopentyl, cyclohexyl, pyrrolidinyl pyrrolyl, piperidino, piperidyl, piperazinyl, pyrazolyl, imidazolyl, phenyl, pyridyl, pyrazinyl, pyridazinyl, pyrimidinyl, benzodioxolyl, naphthyl, indolyl, isoindolyl, quinolyl, isoquinolyl, and dihydroisoquinolyl,
in any of which the carbocyclic or heterocyclic rings may optionally be substituted with 1 to 3 substituents selected from the group consisting of hydroxy, halogen, nitro, cyano, amino, N-(C₁₋₆alkyl)amino, N,N-di(C₁₋₆alkyl)amino, (C₁₋₆)alkylthio, phenyl, phenoxy, benzyl, naphthyl,
(C₁₋₆)alkyl optionally substituted by mono-, di- or tri-halogen, and
(C₁₋₆)alkoxy optionally substituted by mono-, di- or tri-halogen,
wherein
R¹¹ represents (C₁₋₆)alkoxy(C₁₋₆)alkylene,
(C₁₋₆)alkyl optionally substituted by R¹⁰¹,
(C₂₋₆)alkenyl optionally substituted by R¹⁰¹,
(C₂₋₆)alkynyl optionally substituted by R¹⁰¹, or
one of the following carbocyclic or heterocyclic rings selected from the group consisting of cyclopropyl, cyclobutyl, cyclopentyl, cyclohexyl, pyrrolidinyl pyrrolyl, piperidino, piperidyl, piperazinyl, pyrazolyl, imidazolyl, phenyl, pyridyl, pyrazinyl, pyridazinyl, pyrimidinyl, benzodioxolyl, naphthyl, indolyl, isoindolyl, quinolyl, isoquinolyl, and dihydroisoquinolyl,
in any of which the carbocyclic or heterocyclic rings may optionally be substituted with 1 to 3 substituents selected from the group consisting of hydroxy, halogen, nitro, cyano, amino, N-(C₁₋₆)alkylamino, N,N-di(C₁₋₆)alkylamino, (C₁₋₆)alkylthio, phenyl, phenoxy, benzyl, naphthyl, (C₁₋₆)alkyl optionally substituted by mono-, di- or tri-halogen, and (C₁₋₆)alkoxy optionally substituted by mono-, di- or tri-halogen,
R¹⁰¹ represents one of the following carbocyclic or heterocyclic rings selected from the group consisting of cyclopropyl, cyclobutyl, cyclopentyl, cyclohexyl, pyrrolidinyl pyrrolyl, phenyl, pyridyl, pyrimidinyl, benzodioxolyl, naphthyl, indolyl, isoindolyl, quinolyl, isoquinolyl, and dihydroisoquinolyl,
in any of which the carbocyclic or heterocyclic rings may optionally be substituted with 1 to 3 substituents selected from the group consisting of hydroxy, halogen, nitro, cyano, amino, N-(C₁₋₆alkyl)amino, N,N-di(C₁₋₆alkyl)amino, (C₁₋₆)alkylthio, phenyl, phenoxy, benzyl, naphthyl, (C₁₋₆)alkyl optionally substituted by mono-, di- or tri-halogen, and (C₁₋₆)alkoxy optionally substituted by mono-, di- or tri-halogen;
R¹² represents one of the following carbocyclic or heterocyclic rings selected from the group consisting of cyclopropyl, cyclobutyl, cyclopentyl, cyclohexyl, pyrrolidinyl pyrrolyl, phenyl, pyridyl, pyrimidinyl, benzodioxolyl, naphthyl, indolyl, isoindolyl, quinolyl, isoquinolyl, and dihydroisoquinolyl,
in any of which the carbocyclic or heterocyclic rings may optionally be substituted with 1 to 3 substituents selected from the group consisting of hydroxy, halogen, nitro, cyano, amino, N-(C₁₋₆)alkylamino, N,N-di(C₁₋₆)alkylamino, (C₁₋₆)alkylthio, phenyl, phenoxy, benzyl, naphthyl, (C₁₋₆)alkyl optionally substituted by mono-, di- or tri-halogen, and (C₁₋₆)alkoxy optionally substituted by mono-, di- or tri-halogen;
R² represents hydrogen, hydroxy, (C₂₋₆)alkenyl, (C₂₋₆)alkynyl, (C₃₋₇)cycloalkyl, pyrimidinyl, indolyl, pyridyl,
(C₁₋₆)alkoxy optionally substituted by amino, N-(C₁₋₆)alkylamino, N,N-di(C₁₋₆)alkylamino or phenyl,
(C₁₋₆)alkyl optionally substituted by phenyl, mono-, di- or tri-halogen, (C₁₋₆) alkylthio or (C₁₋₆)alkylsulfonyl,
phenyl optionally substituted by halogen, hydroxy, nitro, amino, N-(C₁₋₆)alkylamino, N-(dihydroimidazolyl)amino, (C₁₋₆)alkyl, or (C₁₋₆)alkoxy optionally substituted by R²¹
wherein
R²¹ represents amino, N-(C₁₋₆)alkylamino, N,N-di(C₁₋₆)alkylamino or morpholino;
R³ represents hydrogen or (C₁₋₆)alkyl optionally substituted by mono-, di- or tri-halogen;
R⁴ represents carboxy, tetrazolyl or N-(hydroxy)aminocarbonyl;
R⁵ represents hydrogen, (C₁₋₆)alkyl, (C₁₋₆)alkoxy, phenyl or pyridinyl;
R⁶ represents hydrogen; and
R⁷ represents hydrogen, methyl or ethyl.

5. The phenyl or heteroaryl amino alkane derivative of the formula (I), its tautomeric or stereoisomeric form, or a salt thereof as claimed in claim 1,
wherein
Ar represents 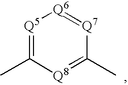
Q⁵ and Q⁷ represent N;
Q⁶ and Q⁸ independently represent CH or CR⁸,
wherein
R⁸ represents fluoro, chloro, amino, nitro, formyl, hydroxymethyl, trifluoromethyl, or methylthio;
Q¹, Q², Q³ and Q⁴ represent CH or CR¹⁰,
wherein
R¹⁰ represents halogen, amino, nitro, formyl, trifluoromethyl, hydroxymethyl, methylthio or benzyloxy;
R¹ represents —OR¹¹, —CH₂NHR¹¹, —C(O)R¹¹, —C(O)NHR¹¹, —SR¹¹, —SOR¹¹, —SO₂R¹¹, —NHR¹¹, —NHC(O)R¹¹, —NHC(O)OR¹¹, —NHC(O)NR¹¹, —NHSO₂R¹¹, hydrogen, hydroxy, halogen, benzodioxolyl, naphthyl,
phenyl optionally substituted with 1 to 3 substituents selected from the group consisting of nitro, (C₁₋₆)alkoxy, (C₁₋₆)alkylthio, phenyl, and phenoxy,
(C₁₋₆) alkyl optionally substituted by anilino, N-(benzyl)amino, indolyl, isoindolyl, quinolyl, isoquinolyl, dihydroisoquinolyl, phenoxyimino, phenyl optionally substituted by halogen, or (C₁₋₆) alkoxy,
wherein
said (C₁₋₆) alkoxy optionally substituted by phenyl, pyridyl, benzodioxolyl, naphthyl, indolyl, isoindolyl, quinolyl, isoquinolyl, or dihydroisoquinolyl,
(C₂₋₆)alkenyl optionally substituted by phenyl,
(C₂₋₆)alkynyl optionally substituted by phenyl,
wherein
R¹¹ represents (C₁₋₆)alkoxy(C₁₋₆)alkylene,
(C₁₋₆) alkyl optionally substituted by R¹⁰¹,
(C₂₋₆)alkenyl optionally substituted by R¹⁰¹,
(C₂₋₆)alkynyl optionally substituted by R¹⁰¹, or
one of the following carbocyclic or heterocyclic rings selected from the group consisting of cyclopropyl, cyclobutyl, cyclopentyl, cyclohexyl, pyrrolidinyl pyrrolyl, phenyl, pyridyl, pyrimidinyl, benzodioxolyl, naphthyl, indolyl, isoindolyl, quinolyl, and dihydroisoquinolyl,
in any of which the carbocyclic or heterocyclic rings may optionally be substituted with 1 to 3 substituents selected from the group consisting of hydroxy, halogen, nitro, cyano, (C₁₋₆)alkylthio, phenyl, phenoxy, benzyl, naphthyl, (C₁₋₆)alkyl optionally substituted by mono-, di- or tri-halogen, or (C₁₋₆)alkoxy optionally substituted by mono-, di- or tri-halogen,
R¹⁰¹ represents one of the following carbocyclic or heterocyclic rings selected from the group consisting of cyclopropyl, cyclobutyl, cyclopentyl, cyclohexyl, pyrrolidinyl pyrrolyl, phenyl, pyridyl, pyrimidinyl, benzodioxolyl, naphthyl, indolyl, isoindolyl, quinolyl, and dihydroisoquinolyl,
in any of which the carbocyclic or heterocyclic rings may optionally be substituted with 1 to 3 substituents selected from the group consisting of hydroxy, halogen, nitro, cyano, (C₁₋₆)alkylthio, phenyl, phenoxy, benzyl, naphthyl, (C₁₋₆)alkyl optionally substituted by mono-, di- or tri-halogen, and (C₁₋₆)alkoxy optionally substituted by mono-, di- or tri-halogen,
R² represents hydrogen, hydroxy, (C₂₋₆)alkenyl, (C₂₋₆)alkynyl, pyrimidinyl, indolyl, pyridyl,
(C₁₋₆)alkoxy optionally substituted by phenyl,
(C₁₋₆)alkyl optionally substituted by phenyl, methylthio, mono-, di- or tri-halogen, or (C₁₋₆) alkylsulfonyl,
phenyl optionally substituted by halogen, hydroxy, nitro, amino, N-(dihydroimidazolyl)amino or (C₁₋₆)alkoxy,
wherein
said (C₁₋₆)alkoxy optionally substituted by amino, N-(C₁₋₆)alkylamino, N,N-di(C₁₋₆)alkylamino, or morpholino;
R³ represents hydrogen or (C₁₋₆)alkyl;
R⁴ represents carboxy, tetrazolyl or N-(hydroxy)aminocarbonyl;
R⁵ represents hydrogen, phenyl or pyridyl;
R⁶ represents hydrogen; and
R⁷ represents hydrogen.

6. The phenyl or heteroaryl amino alkane derivative of the formula (I), its tautomeric or stereoisomeric form, or a salt thereof as claimed in claim 1,
wherein
Ar represents 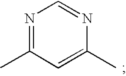
Q¹, Q², Q³ and Q⁴ represent CH;
R¹ represents hydrogen, hydroxy, halogen, benzodioxolyl, naphthyl, cyclopropylmethoxy, cyclobutylmethoxy, cyclopentylmethoxy, cyclohexylmethoxy, cyclopentylcarbonyl, cyclohexylcarbonyl, pyrrolidinylmethoxy, pyrrolidinylethoxy, phenoxy, benzyloxy, fluorobenzyloxy, difluorobenzyloxy, hydroxybenzyloxy, methoxybenzyloxy, dimethoxybenzyloxy, 1H-pyrrolylmethoxy, 1H-pyrrolylethoxy, pyridinyloxy, trifluorometylpyridinyloxy, pyridinylmethoxy, phenylethoxy, pyridinylethoxy, phenylpropoxy, cyanopyridinyloxy, pyrimidinyloxy, trifluoromethylpyrimidinyloxy, quinolinyloxy, benzoyl, fluorobenzoyl, chlorobenzoyl, anilinocarbonyl, benzylamino, benzoylamino, phenylacetylamino, phenylsulfonylamino, fuluoro phenylsulfonylamino, cyclopropylmethylamino, anilinomethyl,
phenyl optionally substituted with 1 to 3 substituents selected from the group consisting of nitro, methoxy, ethoxy, methylthio, phenyl, and phenoxy,
(C₁₋₆)alkyl optionally substituted by anilino, N-(benzyl)amino, indolyl, isoindolyl, quinolyl, isoquinolyl, dihydroisoquinolyl, phenoxy, phenoxyimino, or phenyl optionally substituted by halogen,
(C₂₋₆)alkenyl optionally substituted by phenyl,
(C₂₋₆)alkynyl optionally substituted by phenyl, or
(C₁₋₆)alkoxy optionally substituted by trifluoro or methoxy;
R² represents hydrogen, (C₂₋₆)alkenyl, (C₂₋₆)alkynyl, pyrimidinyl, indolyl, pyridyl,
(C₁₋₆)alkoxy optionally substituted by phenyl,
(C₁₋₆)alkyl optionally substituted by phenyl, methylthio, mono-, di- or tri-halogen, or (C₁₋₆)alkylsulfonyl,
phenyl optionally substituted by halogen, hydroxy, nitro, amino, N-(dihydroimidazolyl)amino or (C₁₋₆)alkoxy optionally substituted by amino, N-(C₁₋₆)alkylamino, N,N-di(C₁₋₆)alkylamino, or morpholino;
R³ represents hydrogen;
R⁴ represents carboxy or tetrazolyl;
R⁵ represents hydrogen;
R⁶ represents hydrogen; and
R⁷ represents hydrogen.

7. The phenyl or heteroaryl amino alkane derivative, its tautomeric or stereoisomeric form, or a salt thereof as claimed in claim 1, wherein said derivative is selected from the group consisting of the following compounds:
3-(2-aminoethoxy)-N-{6-[4-(benzyloxy)phenyl]pyrimidin-4-yl}phenylalanine;
4-chloro-N-{6-[4-(cyclopropylmethoxy)phenyl]pyrimidin-4-yl}phenylalanine;
N-(6-{4-[(2-fluorobenzyl)oxy]phenyl}pyrimidin-4-yl)phenylalanine;
N-(6-{4-[(3,5-difluorobenzyl)oxy]phenyl}pyrimidin-4-yl)-3-pyridin-2-yl-alanine;
N-(6-{4-[(3,5-difluorobenzyl)oxy]phenyl}pyrimidin-4-yl)norleucine;
N-(6-{4-[(3,5-difluorobenzyl)oxy]phenyl}pyrimidin-4-yl)phenylalanine;
N-(6-{4-[(3,5-dimethoxybenzyl)oxy]phenyl}pyrimidin-4-yl)-3-pyridin-2-yl-alanine;
N-(6-{4-[(3,5-dimethoxybenzyl)oxy]phenyl}pyrimidin-4-yl)norleucine;
N-(6-{4-[(3,5-dimethoxybenzyl)oxy]phenyl}pyrimidin-4-yl)phenylalanine;
N-(6-{4-[(3-fluorobenzyl)oxy]phenyl}pyrimidin-4-yl)-3-pyridin-2-yl-alanine;
N-(6-{4-[(3-fluorobenzyl)oxy]phenyl}pyrimidin-4-yl)phenylalanine;
N-(6-{4-[(3-methoxybenzyl)oxy]phenyl}pyrimidin-4-yl)-3-pyridin-2-yl-alanine;
N-(6-{4-[(3-methoxybenzyl)oxy]phenyl}pyrimidin-4-yl)norleucine;
N-(6-{4-[(3-methoxybenzyl)oxy]phenyl}pyrimidin-4-yl)phenylalanine;
N-(6-{4-[(4-fluorobenzyl)oxy]phenyl}pyrimidin-4-yl)phenylalanine;
N-(6-{4-[2-(1H-pyrrol-1-yl)ethoxy]phenyl}pyrimidin-4-yl)phenylalanine;
N-[6-(3′-methoxybiphenyl-4-yl)pyrimidin-4-yl]phenylalanine;
N-[6-(4′-methoxybiphenyl-4-yl)pyrimidin-4-yl]phenylalanine;
N-(6-{4-(1,3-benzodioxol-5-yl)phenyl]pyrimidin-4-yl}phenylalanine;
N-{6-[4-(2-phenylethoxy)phenyl]pyrimidin-4-yl}-3-pyridin-2-yl-alanine;
N-{6-[4-(2-phenylethoxy)phenyl]pyrimidin-4-yl}phenylalanine;
N-{6-[4-(benzyloxy)-3-fluorophenyl]pyrimidin-4-yl}-3-pyridin-2-yl-alanine;
N-{6-[4-(benzyloxy)-3-fluorophenyl]pyrimidin-4-yl}phenylalanine;
N-{6-[4-(benzyloxy)phenyl]-5-fluoropyrimidin-4-yl}phenylalanine;
N-{6-[4-(benzyloxy)phenyl]pyrimidin-4-yl}-3-(2-morpholin-4-ylethoxy)phenylalanine;
N-{6-[4-(benzyloxy)phenyl]pyrimidin-4-yl}-3-[2-(dimethylamino)ethoxy]phenylalanine;
N-{6-[4-(benzyloxy)phenyl]pyrimidin-4-yl}-3-hydroxyphenylalanine;
N-{6-[4-(benzyloxy)phenyl]pyrimidin-4-yl}-3-pyridin-2-yl-alanine;
N-{6-[4-(benzyloxy)phenyl]pyrimidin-4-yl}-4-chlorophenylalanine;
N-{6-[4-(benzyloxy)phenyl]pyrimidin-4-yl}-4-fluorophenylalanine;
N-{6-[4-(benzyloxy)phenyl]pyrimidin-4-yl}-norleucine;
N-{6-[4-(benzyloxy)phenyl]pyrimidin-4-yl}-phenylalanine;
N-{6-[4-(benzyloxy)phenyl]pyrimidin-4-yl}tryptophan;
N-{6-[4-(benzyloxy)phenyl]pyrimidin-4-yl}tyrosine;
N-{6-[4-(cyclopropylmethoxy)phenyl]pyrimidin-4-yl}-4-fluorophenylalanine;
N-{6-[4-(cyclopropylmethoxy)phenyl]pyrimidin-4-yl}-phenylalanine;
N-{6-[4-(phenoxymethyl)phenyl]pyrimidin-4-yl}phenylalanine;
N-{6-[4-(phenylethynyl)phenyl]pyrimidin-4-yl}phenylalanine;
N-{6-[4-(pyridin-3-ylmethoxy)phenyl]pyrimidin-4-yl}phenylalanine; and
N-{6-[6-(benzyloxy)pyridin-3-yl]pyrimidin-4-yl}phenylalanine.

8. The phenyl or heteroaryl amino alkane derivative, its tautomeric or a salt thereof as claimed in claim 1, wherein said derivative is selected from the group consisting of the following compounds:
N-{6-[4-(benzyloxy)phenyl]pyrimidin-4-yl}-3-pyridin-2-yl-D-alanine;
N-{6-[4-(benzyloxy)phenyl]pyrimidin-4-yl}-D-norleucine;
N-{6-[4-(benzyloxy)phenyl]pyrimidin-4-yl}-D-phenylalanine; and
N-{6-[4-(cyclopropylmethoxy)phenyl]pyrimidin-4-yl}-D-phenylalanine.

9. A pharmaceutical composition comprising the phenyl or heteroaryl amino alkane derivative, its tautomeric or stereoisomeric form, or a physiologically acceptable salt thereof as claimed in claim 1 as an active ingredient, and a pharmaceutically acceptable carrier.

10. (canceled)

11. The pharmaceutical composition as claimed in claim 9, wherein the phenyl or heteroaryl amino alkane derivative, its tautomeric or stereoisomeric form, or a physiologically acceptable salt thereof is an IP receptor antagonist.

12. A method for prophylaxis and/or treatment of a urological disorder comprising administering to a subject in need thereof an effective amount of a compound of claim 1.

13. A method for prophylaxis and/or treatment of pain comprising administering to a subject in need thereof an effective amount of a compound of claim 1.

14. A method for prophylaxis and/or treatment of hypertension comprising administering to a subject in need thereof an effective amount of a compound of claim 1.

15. A method for prophylaxis and/or treatment of hemophilia and hemorrhage comprising administering to a subject in need thereof an effective amount of a compound of claim 1.

16. A method for prophylaxis and/or treatment of inflammation comprising administering to a subject in need thereof an effective amount of a compound of claim 1.

17. (canceled)

18. (canceled)

19. (canceled)

20. (canceled)

21. (canceled)

22. A method for controlling a urological disorder in a human or animal comprising administration of an IP receptor-antagonistically effective amount of at least one compound according to claim 1.
